(19) United States
(12) Patent Application Publication
(10) Pub. No.: US 2017/0290889 A1
(43) Pub. Date: Oct. 12, 2017

(54) METHODS AND COMPOSITIONS FOR TREATING DYSBIOSIS AND GASTROINTESTINAL AND INFLAMMATORY DISORDERS

(71) Applicant: New York University, New York, NY (US)

(72) Inventors: P'ng Loke, Leonia, NJ (US); Kenneth Cadwell, Forest Hills, NY (US); Deepshika Ramanan, New York, NY (US); Rowann Bowcutt, London (GB)

(73) Assignee: New York University, New York, NY (US)

(21) Appl. No.: 15/484,555

(22) Filed: Apr. 11, 2017

Related U.S. Application Data
(60) Provisional application No. 62/320,892, filed on Apr. 11, 2016.

(51) Int. Cl. A61K 38/20; A61K 35/62; A61K 35/74; A61K 38/17

(52) U.S. Cl. CPC A61K 38/20; A61K 38/2026; A61K 38/2086; A61K 38/1735; A61K 35/62; A61K 35/74

(57) ABSTRACT

The present invention relates to the use of type 2 cytokines and mucins for increasing the amount or activity of bacterial species of the Clostridia class in the gastrointestinal tract, for treating dysbiosis in the gastrointestinal tract, for treating gastrointestinal and inflammatory disorders, and for promoting wound healing in the gastrointestinal tract.

### STATEMENT REGARDING FEDERALLY SPONSORED RESEARCH

Research and development leading to certain aspects of the present invention were supported, in part, by Grant No. 5R01DK103788-02 awarded by the National Institutes of Health/NIDDK. Accordingly, the U.S. government may have certain rights in the invention.

### SEQUENCE LISTING

The instant application contains a Sequence Listing which has been submitted electronically in ASCII format and is hereby incorporated by reference in its entirety. Said ASCII copy, created on Apr. 6, 2017, is named 243735_000177_SL.txt and is 18,884 bytes in size.

### FIELD OF THE INVENTION

The present invention relates to the use of type 2 cytokines and mucins for increasing the amount or activity of bacterial species of the Clostridia class in the gastrointestinal tract, for treating dysbiosis in the gastrointestinal tract, for treating gastrointestinal and inflammatory disorders, and for enhancing wound healing in the gastrointestinal tract.

### BACKGROUND OF THE INVENTION

Dramatic increases in the incidence of inflammatory bowel disease (IBD) in the developed world point towards alterations in the environment, including changes to the gut microbiota (1) and decreased exposure to intestinal parasites such as helminths (2). Evidence supporting a central role of the microbiota in the pathogenesis of IBD has led to a growing interest in defining the symbiotic relationship between the host and specific microbial species (3). Symbiotic relationships described in insects that develop to defend against environmental hazards (defensive symbiosis) (4) may be applicable to host-microbiota interactions. For example, specific bacterial taxa found within the human gut microbiota likely mediate resistance to antibiotic-associated diarrhea caused by Clostridium difficile (5). Loss of beneficial members of the microbiota potentially contribute to chronic inflammatory diseases as well. Also, helminths and the gut microbiota have co-evolved with their mammalian hosts, but the mechanisms of these interactions and the consequence of decreased exposure to intestinal helminths remain unclear.

Dysregulation of the gut microbiota, also known as dysbiosis is associated with many types of autoimmune inflammatory diseases. Methods for reversing dysbiosis may be beneficial for improving symptoms associated with inflammatory diseases.

The human microbiota represents about 90% of the cells in the human body (Savage et al., 1977, Ann. Rev. Microbiol., 31:107-33). Bacterial communities of mammalian microbiota have co-evolved with hosts and have a complex, bidirectional interaction with the immune system (Hooper et al., 2012, Science, 336:1268-73). The interaction involves microbes, their metabolites, epithelial cells, and cells of the adaptive and innate immune systems. Altering specific immunological components can cause significant effects on the microbiota. Multiple species of commensal microorganisms are harbored in the gastrointestinal (GI) tract of mammals, where they influence the development of the mucosal immune system leading to enhancement of protective functions of the mucous membranes and enabling the host to mount robust immune responses against pathogenic microbes invading the body, while staying non-responsive to dietary antigens and harmless microbes

Abnormality in the regulation of cross-talk between commensal bacteria and the immune system (GI dysbiosis) may lead to inflammatory and gastrointestinal conditions such as inflammatory bowel disease (IBD) ulcerative colitis, or Crohn's disease (U.S. Patent Appl. Pub. No. 20140341921 and references cited therein). Recent studies have shown that the presence of some species of intestinal microbiota influences the differentiation of regulatory T cells (Treg) which help maintain homeostasis of the immune system. For example, Atarashi et al. (Nature, 2013, 500:232-238) isolated 17 strains within Clostridia clusters XIVa, IV and XVIII from a human faecal sample and suggested that these strains affect Treg cell differentiation, accumulation and function in the mouse colon.

### SUMMARY OF THE INVENTION

As specified in the Background Section, there is a great need in the art to identify new approaches to treating dysbiosis in the gastrointestinal (GI) tract, treating GI and inflammatory disorders, and for promoting wound healing in the GI tract. The present invention addresses this and other needs by providing methods based on the use of type 2 cytokines and mucins to increase the amount or activity of bacterial species of the Clostridia class in the GI tract.

In one aspect, the invention provides a method for increasing the amount or activity of one or more bacterial species of the Clostridia class in the gastrointestinal tract of a subject comprising administering to said subject a therapeutically effective amount of a type 2 cytokine and/or a mucin.

In another aspect, the invention provides a method for treating dysbiosis in the gastrointestinal tract of a subject in need thereof, wherein the dysbiosis is associated with a decrease in the amount or activity of one or more bacterial species of the Clostridia class in the gastrointestinal tract of the subject, said method comprising administering to said subject a therapeutically effective amount of a type 2 cytokine and/or a mucin.

In yet another aspect, the invention provides a method for treating a gastrointestinal or inflammatory disorder in a subject in need thereof, which disorder can be treated by increasing the amount or activity of one or more bacterial species of the Clostridia class in the gastrointestinal tract of the subject, said method comprising administering to said subject a therapeutically effective amount of a type 2 cytokine and/or a mucin. Non-limiting examples of disorders treatable by the method of the invention include, e.g., inflammatory bowel disease (IBD), ulcerative colitis, Crohn's disease, irritable bowel syndrome (IBS), sprue, autoimmune arthritis, rheumatoid arthritis, Type I diabetes, multiple sclerosis (MS), amyotrophic lateral sclerosis (ALS), graft vs. host disease, osteoarthritis, juvenile chronic arthritis, Lyme arthritis, psoriatic arthritis, reactive arthritis, spondy loarthropathy, systemic lupus erythematosus (SLE), insulin dependent diabetes mellitus, thyroiditis, asthma, psoriasis, dermatitis scleroderma, atopic dermatitis, graft versus host disease, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, septic shock, toxic shock syndrome, sepsis syndrome, cachexia, acquired immunodeficiency syndrome, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrome, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondy-loarhopathy, atheromatous disease/arteriosclerosis, allergic colitis, atopic allergy, food allergies such as peanut allergy, tree nut allergy, egg allergy, milk allergy, soy allergy, wheat allergy, seafood allergy, shellfish allergy, or sesame seed allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondy litis associated lung disease, vasculitic diffuse lung disease, haemosiderosis associated lung disease, drug-induced interstitial lung disease, radiation fibrosis, bronchiolitis obliterans, idiopathic pulmonary fibrosis, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, multiple sclerosis (all subtypes), insulindependent diabetes mellitus, sympathetic ophthalmia, pulmonary hypertension secondary to connective tissue disease, Goodpasture's syndrome, pulmonary manifestation of polyarteritis nodosa, acute rheumatio fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, allergic rhinitis (pollen allergies), anaphylaxis, pet allergies, latex allergies, drug allergies, allergic rhinoconjuctivitis, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis, diarrhea, colon cancer, cystic fibrosis, celiac disease, Type 2 diabetes, and autism-related immunopathologies.

In a further aspect, the invention provides a method for promoting a wound healing in the gastrointestinal tract of a subject in need thereof comprising administering to said subject a therapeutically effective amount of a type 2 cytokine and/or a mucin.

In one embodiment of any of the above methods, the method further comprises administering to the subject bacteria of the Clostridia class (e.g., from one or more different species and administered, e.g., in the form of live bacterial cells, conditionally lethal bacterial strains, killed bacterial cells, spores, or bacterially derived products). In one specific embodiment, said bacteria of the Clostridia class are from one or more human-derived commensal bacterial species belonging to Clostridium Cluster IV, XIVa, or XVIII. In one specific embodiment, said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense. In another specific embodiment, said bacteria of the Clostridia class are from one or more species selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLG055, Erysipelotrichaceae bacterium 2_2_44A, Clostridium indolis, Anaerostipes caccae, Clostridium bolteae, Lachnospiraceae bacterium DJF_{—VP}30, Lachnospiraceae bacterium 3_1_57 FAA_CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662. In another specific embodiment, said bacteria of the Clostridia class are from one or more species selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLGO55, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA_CT1, Clostridium sp. 316002/08, and Clostridiales bacterium 1_7_47 FAA. In one specific embodiment, said bacteria of the Clostridia class are administered in the form selected from the group consisting of live bacterial cells, conditionally lethal bacterial strains, killed bacterial cells, spores, and bacterially-derived products. In one specific embodiment, said bacteria of the Clostridia class are administered together with (i) a carrier and/or buffering agent and/or (ii) one or more prebiotic agents which enhance growth or activity of said bacteria. In one specific embodiment, (i) said type 2 cytokine and/or a mucin and (ii) said bacteria of the Clostridia class are administered simultaneously (e.g., in one composition or in two or more separate compositions). In another specific embodiment, (i) said type 2 cytokine and/or a mucin and (ii) said bacteria of the Clostridia class are administered sequentially.

Non-limiting examples of type 2 cytokines useful in any of the methods of the invention include, e.g., IL-13, IL-4, IL-22, IL-25, IL-33, and thymic stromal lymphopoietin (TSLP). In one embodiment of any of the methods of the invention, the type 2 cytokine is a fusion protein comprising an amino acid sequence of a mature type 2 cytokine protein and CH₂ and CH₃ domains of Fc region of IgG. Non-limiting examples of useful fusion proteins include, e.g.:

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTFRSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

In one specific embodiment of any of the methods of the invention, the method comprises administering two or more type 2 cytokines (e.g., administered simultaneously [e.g., in one composition or in two or more separate compositions] or sequentially).

Mucin useful in any of the methods of the invention can comprise, e.g., one or more molecules selected from the group consisting of MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, and MUC21. In one specific embodiment, mucin comprises MUC2.

In one embodiment of any of the methods of the invention, the method comprises administering a type 2 cytokine and a mucin (e.g., administered simultaneously [e.g., in one composition or in two or more separate compositions] or sequentially).

In one embodiment of any of the methods of the invention, the therapeutically effective amount of type 2 cytokine and/or mucin corresponds to the minimum dose required for the induction of M2 macrophages in the gastrointestinal tract of the subject (e.g., as detected by monitoring the expression of one or more of PD-L2, CD301, CD206, Arg1, Ym1/Chi3l3, and Fizzl/Relma). In one embodiment of any of the methods of the invention, the therapeutically effective amount of type 2 cytokine and/or mucin corresponds to the minimum dose required for the decrease of Bacteroides vulgatus abundance in the stool of the subject (e.g., at least 90% decrease, preferably at least 99% decrease). In one embodiment of any of the methods of the invention, the therapeutically effective amount of type 2 cytokine and/or mucin corresponds to the minimum dose required for the increase of the abundance of said Clostridial species in the stool of the subject (e.g., at least 100% increase).

Non-limiting examples of useful administration routes for type 2 cytokines and mucins in the methods of the invention include, e.g., oral, rectal, fecal (e.g., by enema), and via naso/orogastric gavage. In one embodiment of any of the methods of the invention, type 2 cytokine and/or mucin is administered systemically. In one embodiment of any of the methods of the invention, type 2 cytokine and/or mucin is administered in the form of nanoparticles. In another embodiment of any of the methods of the invention, type 2 cytokine and/or mucin is administered in the form of a bacterial, yeast or viral strain engineered to produce such type 2 cytokine and/or mucin. In one specific embodiment, such bacterial strain is a Lactobacillus strain.

In one embodiment of any of the methods of the present invention, the method results in increasing the amount or activity of one or more bacterial species of the Clostridia class in the gastrointestinal tract of a subject, wherein the bacterial species of the Clostridia class is human-derived commensal bacterial species belonging to Clostridium Cluster IV, XIVa, or XVIII. In one embodiment of any of the methods of the present invention, the method results in increasing the amount or activity of one or more bacterial species of the Clostridia class in the gastrointestinal tract of a subject, wherein the bacterial species of the Clostridia class is selected from the group consisting of Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense. In one embodiment of any of the methods of the present invention, the method results in increasing the amount or activity of one or more bacterial species of the Clostridia class in the gastrointestinal tract of a subject, wherein the bacterial species of the Clostridia class is selected from the group consisting of Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLGO55, Erysipeltrichaceae bacterium 2_2_44A, Clostridium indolis, Anaerostipes caccae, Clostridium bolteae, Lachnospiraceae bacterium DJF_VP30, Lachnospiraceae bacterium 3_1_57 FAA_CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662. In one embodiment of any of the methods of the present invention, the method results in increasing the amount or activity of one or more bacterial species of the Clostridia class in the gastrointestinal tract of a subject, wherein the bacterial species of the Clostridia class is selected from the group consisting of Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLG055, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA_CT1, Clostridium sp. 316002/08, and Clostridiales bacterium 1_7_47 FAA.

In one embodiment of any of the methods of the invention, the method further comprises administering an effective amount of one or more Helminth species. Non-limiting examples of useful Helminth species include, e.g., Trichuris trichiura, Schistosoma mansoni, Ancylostoma duodenale, Necator americanus, Fasciola hepatica, and Taenia saginata.

In one embodiment of any of the methods of the present invention, the subject is human.

In conjunction with the above-described methods, the invention also provides compositions comprising two or more components selected from the group consisting of (i) a type 2 cytokine, (ii) a mucin, (iii) bacteria of the Clostridia class (e.g., in the form of live bacterial cells, conditionally lethal bacterial strains, killed bacterial cells, spores, or bacterially derived products), (iv) bacteria (e.g., Lactobacillus), yeast or virus expressing a type 2 cytokine, (v) yeast or virus expressing a mucin, and (vi) a helminth (e.g., Trichuris trichiura, Schistosoma mansoni, Ancylostoma duodenale, Necator americanus, Fasciola hepatica, or Taenia saginata).

In one embodiment, said bacteria of the Clostridia class in the compositions of the invention are from one or more different species. In one embodiment, said bacteria of the Clostridia class are from one or more human-derived commensal bacterial species belonging to Clostridium Cluster IV, XIVa, or XVIII. In one embodiment, said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense. In one embodiment, said bacteria of the Clostridia class are from one or more species selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLG055, Erysipelotrichaceae bacterium 2_2_44A, Clostridium indolis, Anaerostipes caccae, Clostridium bolteae, Lachnospiraceae bacterium DJF_VP306, Lachnospiraceae bacterium 3_1_57 FAA CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662. In one embodiment, said bacteria of the Clostridia class are from one or more species selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLG055, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA_CT1, Clostridium sp. 316002/08, and Clostridiales bacterium 1_7_47 FAA.

Non-limiting examples of type 2 cytokines useful in the compositions of the invention include, e.g., IL-13, IL-4, IL-22, IL-25, IL-33, and thymic stromal lymphopoietin (TSLP). In one specific embodiment, the composition of the invention comprises two or more type 2 cytokines. In one embodiment, the type 2 cytokine is a fusion protein comprising an amino acid sequence of a mature type 2 cytokine protein and CH₂ and CH₃ domains of Fc region of IgG. Non-limiting examples of useful fusion proteins include, e.g.:

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTFRSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

Mucin useful in the compositions of the invention can comprise, e.g., one or more molecules selected from the group consisting of MUC1, MUC2, MUC3A, MUC3B, MUC4, MUCSAC, MUCSB, MUC6, MUC7, MUC8, MUC12, MUC13, MUC15, MUC16, MUC17, MUC19, MUC20, and MUC21. In one specific embodiment, mucin comprises MUC2.

In one specific embodiment, the composition of the invention comprises a type 2 cytokine and/or a mucin associated with nanoparticles.

The compositions of the invention may comprise (i) a carrier and/or buffering agent and/or (ii) one or more prebiotic agents which enhance growth or activity of one or more bacteria present in the composition. The compositions of the invention can be formulated for any administration route (e.g., oral, rectal, fecal (e.g., by enema), or via naso/oro-gastric gavage).

These and other aspects of the present invention will be apparent to those of ordinary skill in the art in the following description, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1H shows that Trichuris muris infection reverses intestinal abnormalities in Nod2^{-/-} mice. (1A-1B) PAS-Alcian blue stained small intestinal sections (1A) and quantification of the number of goblet cells displaying normal morphology per villi (1B) from uninfected and T. muris infected WT and Nod2^{-/-} mice (n≧7 per genotype). (1C-1D) Immunofluorescence (IF) analysis of Reg3β in small intestine (1C) and quantification of the mean fluorescence intensity (MFI) (1D) of above mice (n≧8 per genotype). (1E) Quantification of the proportion of CD8 +intra-epithelial lymphocytes (IELs) expressing IFN-γ by flow cytometry (n≧11 per genotype). (1F-1H) Quantification of weight loss (1F), H&E-stained small intestinal sections (1G), and quantification of pathology (48) (1H), following piroxicam treatment of uninfected and T. muris infected WT and Nod2^{-/-} mice. Asterisk denotes an abscess in (1G). (n≧7 per genotype). *p<0.05, **“p<0.01, and ****p<0.0001 by ANOVA with Holm-Sidak multiple comparisons test for (1B), (1D), (1E), (1F), and (1H). Scale bar represents 50 μm in (1A), 100 μm in (1C) and (1G). Data are represented as mean ± SEM in (1F), each data point represents an individual mouse and bar denotes mean in (1B), (1D), (1E), and (1H), from at least two independent experiments.

FIG. 2A-2M shows that helminth infection inhibits Bacteroides vulgatus colonization through a type-2 immune response. (2A) Quantification of B. vulgatus colony forming units (cfu) in stool from T. muris infected WT and Nod2^{-/-} mice (n≦10 per genotype). (2B) Quantification of pSTAT6 staining in the small intestine of T. muris infected WT and Nod2^{-/31} mice (n≧3 per genotype). (2C) Quantification of B. vulgatus in stool from T. muris infected WT (Nod2^{-/-}→WT) and Stat6^{-/-} (Nod2^{-/-→Stat}6^{-/-}) mice reconstituted with Nod2^{-/-} bone marrow (BM). Both WT and Stat6^{-/-} chimeric mice were gavaged with B. vulgatus to ensure equal colonization before T. muris infection (n≧5 per genotype). (2D) Quantification of the total number of small intestinal lamina propria CD4⁺ T cells expressing IL-13 in uninfected and T. muris infected Nod2^{-/-} mice (n≧4 per genotype). (2E) Fold-increase in the number CD4⁺ T cells producing IFN-γ, IL-13, or IL-10 in the small intestinal lamina propria of T. muris infected WT and Nod2^{-/31} mice, normalized to uninfected mice (n≧4 per genotype). (2F) Quantification of B. vulgatus associated with small intestinal tissue of uninfected, T. muris infected, and H. polygyrus infected Nod2^{-/-} mice (n≧10 per genotype). (2G-2H) Quantification of goblet cells displaying normal morphology per villi (2G) and total number of small intestinal lamina propria CD4⁺ T cells expressing IL-13 (2H) in uninfected and H. polygyrus infected WT and Nod2^{-/-} mice (n≧3 per genotype). (2I-2J) Quantification of B. vulgatus in small intestinal tissue (2I), and goblet cells displaying normal morphology (2J) in H. polygyrus infected Nod2^{-/-} mice treated with antibody to IL-13 or isotype control (n=6 per genotype). (2K-2L) Quantification of goblet cells displaying normal morphology (2K) and B. vulgatus in stool (2L) in Nod2^{-/-} mice treated with recombinant IL-13 or PBS (n=8 per genotype). (2M) Pathway analysis based on GO terms of genes upregulated in Nod2^{-/-} mice treated with recombinant IL-13 compared to PBS controls. *p<0.05, **p<0.01, ***p<0.001, and ****p<0.0001 by ANOVA with Holm-Sidak multiple comparisons test for (2A), (2B), (2G) and (2H), and unpaired t-test for (2C), (2D), (2F), and (2I)-(2L). Data are represented as mean±SEM in (2A), (2B), (2C), (2E), and (2L), each data point represents an individual mouse and bar denotes mean in (2D), and (2F)-(2K), from at least two independent experiments, excluding the generation of bone marrow chimeras in (2C).

FIG. 3A-3G demonstrates that inhibition of Bacteroides vulgatus is associated with expansion of Clostridiales following helminth infection. (3A) Quantification of B. vulgatus in stool harvested from uninfected and T. muris infected Nod2^{-/-} mice co-housed for the duration of the experiment (n≧4). (3B) Relative abundance of taxonomic groups in response to T. muris infection in the stool of WT and Nod2^{-/-} mice as determined by 16S sequencing (n≧5 per genotype). (3C) Supervised analysis of 16S sequencing data with LDA effect size (LEfSe) comparing Nod2^{-/-} mice at D0 and D21 post infection with T. muris using an LDA threshold score of 4 (n≧5). (3D) LEfSE analysis to determine alterations to the stool microbiota after recombinant IL-13 treatment of Nod2^{-/-} mice using an LDA threshold score of 4 (n≧5). (3E) Quantification of B. vulgatus in stool harvested from Nod2^{-/-} mice gavaged with sterile broth, L. johnsonii, or a mix of 17 Clostridiales and Erysipelotrichales strains (n≧3). (3F-3G) Quantification of Clostridium species (Clostridiales #28) (3F) or B. vulgatus (3G) in the presence of varying concentrations of pig intestinal mucin or vehicle in the culture media. ***p<0.001, ****p<0.0001 by ANOVA with Holm-Sidak multiple comparisons test for (3E), and (3F). Data are represented as mean±SEM from at least two independent experiments.

FIG. 4A-4J demonstrates that helminth colonization in humans is associated with a decrease in Bacteroidales and an increase in Clostridiales. (4A) Beta diversity plots of gut microbiota from urban controls in Kuala Lumpur (red dots) or the Orang Asli (blue dots). (4B) Relative abundance of a dominant Bacteroides OTU in the Orang Asli and urban controls. (4C-4F) Supervised LEfSE analysis (4C), relative abundance of Bacteroidales (4D) and Clostridiales (4E), and alpha diversity as Observed OTUs (4F) of the Orang Asli stool microbiota pre and post treatment with Albendazole. (n=19 for urban controls and 55 Orang Asli. n=53 for deworming experiments). (4G) Partial Least Squares regression biplots examining within subject variances with repeated measures design to identify bacterial taxa associated with Trichuris trichiura worm burden (intensity of spots). Red arrows are Clostridiales taxa and green arrows are Bacteroidales taxa. (4H) Specific OTUs identified to be positively (Dialister) or negatively (Prevotella) associated with changes to T. trichiura egg burdens. (4I-4J) Microbial network inference demonstrating an antagonistic relationship between Clostridiales and Bacteroidales communities from the Human Microbiome Project (4I) and the pediatric IBD RISK cohort (4J). The node diameter is proportional to the geometric mean of the OTU's relative abundance. Numerical values on the edges represent the fraction of edges that are either majority positive (Green) or majority negative (Red). Also see FIG. S10. ****p<0.0001 by unpaired t-test in (4B), and paired t-test in (4D)-(4F).

FIG. 5A-5E shows that T. muris can reverse intestinal abnormalities in Nod2^{-/-} mice. (5A) Quantification of T. muris in the cecum and large intestine of WT and Nod2^{-/-} mice 35 days post infection demonstrates successful colonization of both genotypes (n=8 mice per genotype). (5B) Representative images and quantification of goblet cells in PAS/Alcian blue stained sections of small intestinal crypts in uninfected WT and Nod2^{-/-} mice. It has been previously described that goblet cell defects in the villi of Nod2^{-/-} small intestine is due to increased IFN-γ production by IELs (6). In contrast to the villi, small intestinal crypts display no differences in goblet cell numbers, likely due to the scarcity of IELs. Scale bar represents 50 μm. (5C) Representative images of PAS/Alcian blue stained sections of the colon demonstrating similar goblet cell numbers in uninfected and T. muris infected WT and Nod2^{-/-} mice. Scale bar represents 100 μm. (5D) Immunofluorescence (IF) analysis of Reg3β in uninfected and T. muris infected WT small intestinal sections. Samples from Nod2^{-/-} mice from the same experiment are in FIG. 1C with the quantification of all four conditions in FIG. 1D. Scale bar represents 100 μm (n≧8 mice per genotype). (5E) Representative flow cytometry plot of CD8+ IELs in uninfected and T. muris infected WT and Nod2^{-/-} mice stained for intracellular IFN-γ and IL-17 expression after stimulation with PMA and ionomycin gated on CD3+ live cells (n≧11 mice per genotype). Data are representative of at least two independent experiments. Each data point represents an individual mouse, bar denotes mean in (5A). Data are represented as mean±SEM for individual colon crypts in (5B).

FIG. 6A-6C demonstrates that chronic T. muris infection induces an increase in IFN-γ producing CD4⁺ T cells in the intestinal lamina propria. (6A-6C) Representative flow cytometry plot (6A) and quantification of CD4+ T cells in the colon (6B) and small intestinal lamina propria (6C) of uninfected and T. muris infected WT and Nod2^{-/-} mice stained for intracellular IFN-γ expression after stimulation with PMA and ionomycin (n≧5 mice per genotype). CD4⁺ T cells in the colon of infected WT and Nod2^{-/-} mice, displayed an increase in IFN-γ production, an observation that is consistent with the effect of chronic T. muris infection of mice on the C57BL/6 background (47). However, in the small intestinal lamina propria, only CD4⁺ T cells in WT mice display an increase in IFN-γ production post T. muris infection. *p>0.05 by ANOVA with Holm-Sidak multiple comparisons test for (6C). Data are representative of at least two independent experiments. Each data point represents an individual mouse, bar denotes mean.

FIG. 7A-7J shows that T. muris protects Nod2^{-/-} mice from piroxicam-induced pathologies. (7A) Representative image comparing the small intestine of uninfected (right) or T muris infected (left) Nod2^{-/-} mice treated with piroxicam. Black arrows denote the presence of live parasites in the cecum of T. muris infected Nod2−/− mice. In contrast to the T. muris infected Nod2^{-/-} or WT mice, or uninfected WT mice, the small intestine from uninfected Nod2^{-/-} mice cannot be straightened without tearing due to fusion of the organ with itself and surrounding tissue within the peritoneal cavity including mesenteric fat and cecum. (7B) Representative image of fusion of the small intestinal wall with surrounding tissue (denoted by black arrow) in uninfected Nod2^{-/-} mice treated with piroxicam. (7C) Table representing quantification of small intestinal bleeding and attachment of the intestine to the surrounding tissue of the peritoneal cavity as in (7A) and (7B) in uninfected and T. muris infected WT and Nod2^{-/-} mice treated with piroxicam. (7D-7J) Representative H&E-stained sections of the small intestine demonstrating the presence of the following pathologies detected at higher frequency in Nod2^{-/-} mice treated with piroxicam: abscesses (higher magnification images show presence of infiltrating neutrophils and plasma cells, fibrin deposition, and cell debris, characteristic of abscesses) (7D), subserosal infiltrates (higher magnification image shows presence of neutrophils and plasma cells infiltrating the subserosa) (7E), villus blunting (7F), and epithelial hyperplasia (7G). Occurrence of these pathologies was used to generate the pathology score in FIG. 1G as described in the Materials Methods section below. (7H-7J) Representative images of H&E-stained sections prepared from fused organs harvested from Nod2^{-/-} mice treated with piroxicam as in (7B) demonstrate intestinal perforation. Mesenteric tissue was observed penetrating into small intestinal tissue (asterisk denotes fusion of the two tissue types) (7H). The presence of intestinal contents was observed in adjoining tissues such as the liver and skin (asterisks denote areas of fusion between the different tissue types; liver tissue surrounding the intestinal contents have increased immune infiltrates and abscesses) (7I) and (7J). **p<0.01, ***p<0.001 by Fisher's exact test for (7C). Image magnification at 2×, 10×, and 40× in (7D); 5×, and 40× in (7E); 5× in (7F), (7G), and 2× in (7H)-(7J). Data are representative of at least two independent experiments.

FIG. 8A-8E demonstrates that T. muris protects piroxicam-treated Nod2^{-/-} mice from disease manifestations. (8A) Representative PAS-Alcian blue-stained sections of the small intestine from uninfected and T. muris infected WT and Nod2^{-/-} mice treated with piroxicam. These images show that uninfected Nod2^{-/-} mice display a decreased presence of mucus (blue staining), which is restored upon T. muris infection, an observation that is consistent with a reduction in bacterial translocation following T. muris infection. Scale bar represents 50 μm. (8B) Spleens from Nod2^{-/-} mice treated with piroxicam were larger compared with those from similarly treated uninfected WT and T. muris infected WT and Nod2^{-/-} mice. (8C-8E) Quantification of bacterial colony forming units (cfu) in spleen (8C), mesenteric lymph nodes (8D), and small intestinal tissue (see Materials and Methods) (8E), in uninfected and T. muris infected WT and Nod2^{-/-} mice treated with piroxicam (n≧3 mice per genotype). *p<0.05, **p<0.01, and ****p<0.0001 by ANOVA with Holm-Sidak multiple comparisons test for (8C-8E). Data are representative of at least two independent experiments. Each data point represents an individual mouse, bar denotes mean.

FIG. 9A-9G shows that T. muris mediated protection from intestinal abnormalities is associated with CD4+ T cells. (9A) Quantification of B. vulgatus in stool of T. muris infected Rag^{-/-} and Nod2^{-/-} Rag^{-/-} mice (n≧4 mice per genotype). (9B) Quantification of T. muris in the cecum and large intestine of Rag^{-/-} and Nod2^{-/-}Rag^{-/-} mice from (9A) demonstrating that the inability to reduce B. vulgatus burden is not due to lack of chronic parasite infection (n≧4 mice per genotype). (9C) Quantification of B. vulgatus in stool of T. muris infected Nod2^{-/-} mice treated with antibody to CD4 or isotype control (n≧4 mice per genotype). (9D) Representative immunohistochemistry staining of pSTAT6 in the small intestine of uninfected and T. muris infected Nod2^{-/-} mice. Quantification shown in FIG. 2B. Black arrows denote pSTAT6 positive cells in the small intestinal villi. Scale bar represents 100 μm. (9E) Quantification of goblet cells displaying normal morphology in the small intestine of T. muris infected WT (Nod2^{-/-}→WT) and Stat6^{-/-} (Nod2^{-/-}→Stat6^{-/-}) mice reconstituted with Nod2^{-/-} bone marrow (BM) shows that Stat6 is necessary for helminth-mediated restoration of goblet cells (n≧3 mice per genotype). (9F) Quantification of CD4+ T cells expressing IL-10 in the small intestinal lamina propria of uninfected and T. muris infected WT and Nod2^{-/-} mice after stimulation with PMA and ionomycin (n≧3 mice per genotype). While infected Nod2^{-/-} mice display a marginal increase, T. muris infected WT mice display a substantial increase in IL-10 producing CD4⁺ T cells, consistent with previous studies (47). (9G) Representative flow cytometry plot of uninfected and T. muris infected WT and Nod2^{-/-} mice stained for Helios and Foxp3 expression in CD4⁺ live T cells, indicating that the number of natural or induced regulatory T cells were not significantly altered in the intestinal tissues of either infected WT or of Nod2^{-/-} mice. *p<0.05, **p<0.01, and ****p<0.0001 by unpaired t-test in (9C) and (9E), and ANOVA with Holm-Sidak multiple comparisons test for (9F). Each data point represents an individual mouse, bar denotes mean in (9B), (9E), and (9F). Data are represented as mean±SEM in (9A) and (9C).

FIG. 10A-10F demonstrates that helminth mediated protection from intestinal abnormalities is associated with the type-2 response. (10A) Representative flow cytometry plot of CD8⁺ IELs in uninfected and H. polygyrus infected WT and Nod2^{-/-} mice stained for intracellular IFN-γ and IL-17 expression after stimulation with PMA and ionomycin gated on CD3+ live cells (n=6 mice per genotype). (10B) Quantification of B. vulgatus in stool of H. polygyrus infected Nod2^{-/-} mice 12 days post infection (n=8 mice per genotype). The partial reduction of B. vulgatus in stool compared to T. muris is probably because H. polygyrus and T. muris occupy different niches in the intestine, small intestine and cecum/colon, respectively. (10C) Representative flow cytometry plots of CD4+ T cells in the small intestinal lamina propria of uninfected and H. polygyrus infected Nod2^{-/-} mice stained for intracellular IL-13 and IL-4 expression after stimulation with PMA and ionomycin gated on CD3⁺ live cells (n≧6 mice per genotype). H. polygyrus induced a greater increase in IL-13⁺ CD4 T cells in the small intestinal LP of WT and Nod2^{-/-} mice compared with T. muris (FIG. 2D), which likely explains the large reduction of tissue-associated B. vulgatus burden following infection by this parasite (FIG. 2F). (10D) Proportion of CD4⁺ T cells expressing IL-10 in the small intestinal lamina propria of H. polygyrus infected WT and Nod2^{-/-} mice after stimulation with PMA and ionomycin 12 days post infection (n≧5 mice per genotype). In contrast to T. muris infection, there were no differences in IL-10 producing CD4⁺ T cells between H. polygyrus infected WT and Nod2^{-/-} mice. (10E) Quantification of B. vulgatus in stool of Nod2^{-/-} mice treated with recombinant IL-4 complex (n≧6 per genotype). (10F) Heat map of the 9 genes that were upregulated in Nod2^{-/-} mice treated with recombinant IL-13 compared to PBS controls. All 9 are associated with M2 macrophages. *p<0.05, and ***p<0.001 by paired t-test for (10B) and (10E), and unpaired t-test for (10D). Each data point represents an individual mouse, bar denotes mean.

FIG. 11A-11B demonstrates that chronic H. polygyrus infection leads to type-2 immunity mediated clearance of B. vulgatus. (11A) Longitudinal quantification of B. vulgatus in stool of H. polygyrus infected Nod2^{-/-} mice (n=4 mice per genotype). (11B) Quantification of CD4+ T cells expressing IL-13 in the small intestinal lamina propria of uninfected and H. polygyrus infected Nod2^{-/-} mice after stimulation with PMA and ionomycin 35 days post infection (n≧3 mice per genotype). **p<0.01, and ****p<0.0001 by ANOVA with Holm-Sidak multiple comparisons test for (11A), and unpaired t-test for (11B). Data are represented as mean ±SEM in (11A). Each data point represents an individual mouse, bar denotes mean in (11B).

FIG. 12A-12I shows that alterations to the microbiota after helminth infection are distinct for WT and Nod2^{-/-} mice. (12A-12B) Schematic for co-housing uninfected Nod2^{-/-} and T muris infected Nod2^{-/-} mice (12A), and uninfected Nod2^{-/-} and T. muris infected WT mice (12B). (12C) Quantification of B. vulgatus in stool harvested from uninfected Nod2^{-/-} mice co-housed with T. muris infected WT mice as in (12A) (n=3). Unlike co-housing with T. muris-infected Nod2^{-/-} mice (FIG. 3A), co-housing with T. muris-infected WT mice does not lead to a reduction in B. vulgatus burden in naïve Nod2^{-/-} mice. (12D) Alpha-diversity in T. muris infected WT and Nod2^{-/-} stool at day 0, 21, and 35 post T. muris infection. (n≧5 per genotype). (12E) LEfSE analysis to determine alterations to the stool microbiota of Nod2^{-/-} mice after recombinant IL-4 treatment using an LDA threshold score of 3. (n≧6 per genotype). (12F) Relative abundance of taxonomic groups in response to T. muris infection in the small intestinal tissue of WT and Nod2^{-/-} mice as determined by 16S sequencing (n≧5 per genotype). (12G) Relative abundance of taxonomic groups in response to H. polygyrus infection in the small intestinal tissue of WT and Nod2^{-/-} mice as determined by 16S sequencing (n≧5 per genotype). A robust expansion of Clostridia is observed with both helminth infections, consistent with 16S analysis of stool. (12H-12I) Quantification of Ruminococcus species (Clostridia mixture species #13) (12H) or Erysipelatoclostridium species (Clostridia mixture species #18) (12I) in the presence of varying concentrations of pig intestinal mucin or vehicle in the culture media. **p<0.01, ****p<0.0001 by ANOVA with Holm-Sidak multiple comparisons test for (12D), (12H), and (12I). Asterisks represent p-value between vehicle control and all concentrations of mucin in (12H) and (12I). Data are represented as mean±SEM from at least two independent experiments.

FIG. 13A-13E demonstrates that Albendazole treatment of the Orang Asli reduces microbial diversity of the gut microbiota and alters the composition of bacterial communities. (13A) Schematic of the sample collection and deworming regimen for the longitudinal study of the microbiota of Orang Asli. (13B) Trichuris trichiura egg burden is reduced post treatment with Albendazole (3 doses, 400 mg/day) despite low cure rates (32.9%). Box and whisker plot shows alteration in eggs per gram of feces as determined by traditional Kato-Katz method. (13C) Reduced alpha-diversity (as Observed OTUs) post treatment with Albendazole in paired analyses. (13D) Rarefaction curves calculated for the Chao 1 index (Left) (p=0.009) and Shannon index (Right) (p=0.003) estimators of alpha diversity show reduced microbial diversity post treatment (red lines) compared to pre treatment (blue lines). (13E) Visualization using a cladogram of bacterial taxa identified to be significantly different between pre-treatment and post-treatment stool samples of the Orang Asli utilizing LEfSe with a log LDA score above 3.00. ***p<0.001, ****p<0.0001 by paired t-test for (13B) and (13C).

FIG. 14A-14G shows that the effects of Albendazole on the gut microbiota of the Orang Asli is associated with Trichuris trichiura egg burden. (14A-14C) Centered log-ratio (clr) transformation of microbial compositions (from 16S sequencing data) followed by spares Partial Least Squares regression (sPLS) analyses was used to compute a model on within-subject changes for dr-transformed microbial community compositions and (log-transformed) Trichuris trichiura egg burdens (14A). Change in Trichuris trichiura egg burden is strongly associated with a minimal set of taxa and the linear combinations of selected microbial taxa showed accurate prediction (r2=0.777) of change Trichuris trichiura egg burdens (ΔTrichuris) and no predictive value for Age (14B). Scatter plots A and B illustrate the relationship between change in egg burdens and Age with the within model predicted responses from the sPLS regression analyses. There is also predicted relationship with Gender (14C), where a receiver operator characteristic (ROC) curve showing the true positive vs. false positive rates over predicting various characteristics points to essentially random predictions at AUC=0.5 (and follows closely the x=y line), whereas perfect prediction would have an AUC=1. Hence, selected taxa were found to accurately predict change in Trichuris trichiura egg burdens (ΔTrichuris), but not Age or Sex despite being included in the model. (14D) Scatterplots illustrating the relationship between specific OTUs identified as the minimal set of taxa predictive of changes in Trichuris trichiura egg burdens. (14E) To separate T. trichiura into “responders” (Blue lines) and “non-responders” (Pink lines) to Albendazole treatment, a cutoff of—delta 0.03 log egg burden was used (requiring the presence of T. trichiura before treatment). (14F-14G) As expected, sPLS-regression accurately associated specific bacterial taxa abundance with changes in T. trichiura burden in the “responders” (R2=0.693) (14F) but not in “non-responders” (14G). These results show that the effects of T. trichiura infection on the microbial communities described are directly driven by changes in worm burden, independently of Age, Gender, and Albendazole treatment.

FIG. 15A-15F shows that inferring microbial networks by SPIEC-EASI reveals an antagonistic relationship between Clostridiales and Bacteroidales. (15A-15F) SPIEC-EASI (SParse InversE Covariance Estimation for Ecological Association Inference) is a statistical method for the inference of microbial ecological networks. This approach addresses the problems of compositional and sparse datasets inherent in 16S based investigations of microbial communities (see Materials and Methods). Application of this method in an unbiased fashion to the gut microbiome data from the Human Microbiome Project (15A) and the American Gut Project (15B) identified a consistent negative association (red line) between Bacteroidales and Clostridiales, as well as a positive association (green line) between Erysipelotrichales and Clostridiales. Networks are visualized with OTU nodes colored by Order, with the node diameter proportional to the geometric mean of the OTU's relative abundance. Numerical values on the edges are the fraction of edges that are either majority positive (Green) or majority negative (Red). In one case there are exactly equal numbers of positive and negative examples (Grey). These positive and negative relationships were also observed in the gut microbiota data from the Orang Asli before deworming treatment (15C) and after deworming treatment (15D). Among IBD patients in the American Gut Project (15E) and the Pediatric RISK Cohort (15F), there is also a strong positive association between Clostridiales and Lactobacillales, which is not observed among the Orang Asli. Whereas there is a strong positive association between Clostridiales and Enterobacteriales among the Orang Asli (15A & 15B), this becomes a negative association among IBD patients in the USA that are part of the American Gut Project (15E) or the RISK Cohort (15F). These results indicate that the antagonistic relationship between Clostridiales and Bacteroidales identified herein with helminth infection mouse models and a small dataset of helminth infected individuals may reflect a general phenomenon reflected in much larger datasets of USA residents. This negative relationship is the most consistently observed negative relationship in all available datasets.

### DETAILED DESCRIPTION OF THE INVENTION

The present invention is based on an unexpected discovery that treatment with type 2 cytokines such as IL-4 and IL-13 induces the expansion of bacteria from the class Clostridia in the GI tract, which can reverse dysbiosis by inhibiting inflammatory bacterial communities associated with the order Bacteroidales. Many of the affected Clostridia species described herein fall, under current categorization rules, in the Clostridiaceae family, and belong to clusters IV, XIVa, and XVIII. Clostridiales are an example of defensive symbionts with an antagonistic interaction with another common commensal bacteria (Bacteroidales), which were consistently observed in all human gut microbiome datasets. Bacteroidales are pathogenic only in susceptible Nod2 deficient hosts and this competition reverses disease pathologies.

The present invention is also based on the unexpected discovery that helminths reduce intestinal inflammatory responses by promoting expansion of protective bacterial communities that inhibit pro-inflammatory bacterial taxa.

### Definitions

Unless defined otherwise, technical and scientific terms used herein have the same meaning as commonly understood by one of ordinary skill in the art to which this invention belongs.

As used herein, the term “type 2 cytokine” refers to cytokines involved in type 2 immune responses. Type 2 immunity promotes antihelminth immunity, suppresses type 1-driven autoimmune disease, neutralizes toxins, maintains metabolic homeostasis, and regulates wound repair and tissue regeneration pathways following infection or injury. Type 2 immunity induces a complex inflammatory response characterized by eosinophils, mast cells, basophils, type 2 innate lymphoid cells, interleukin-4 (IL-4)-and/or IL-13-conditioned macrophages, such as M2 macrophages, and T helper 2 (T_{H}2) cells, which are crucial to the pathogenesis of many allergic and fibrotic disorders. Non-limiting examples of type 2 cytokines useful in the methods and compositions of the present invention include IL-13, IL-4, IL-22, IL-25, IL-33, and thymic stromal lymphopoietin (TSLP).

As used herein, the term “mucin” refers to the family of high molecular weight, heavily glycosylated proteins (also referred to as glycoconjugates, mucopolysaccharides or glycoproteins) produced by epithelial tissues in most animals, including humans and other mammals. Mucins are known to form gels and therefore are involved in a wide range of cellular processes and pathways, such as lubrication, forming mucosal barriers, cell signaling, and forming chemical barriers. Mucins serve an important innate immune function by detoxifying noxious molecules and by trapping and removing pathogens and particulates. Mucin glycoproteins are large, heavily glycosylated proteins with a defining feature of tandemly repeating sequences of amino acids rich in serine, proline and threonine, the linkage sites for large carbohydrate structures such as O-linked oligosaccharide chains. Mucin in the gastrointestinal tract forms a viscoelastic mucous gel layer on the luminal surface of the gastrointestinal tract that acts as a protective barrier against the harsh luminal environment. Gastrointestinal mucins may contain sulfur groups and/or sialyl groups, and such modified mucins are termed sulfomucins and sialomucins, respectively. Non-limiting examples of mucins encompassed by the present invention include MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, and MUC21, and derivatives thereof.

As used herein, the term “M2 macrophage” refers to macrophages that decrease inflammation and encourage tissue repair and have the ability to metabolize arginine to ornithine and produce anti-inflammatory cytokines (e.g., IL-10, TGF-beta and IL-12). M2 macrophages have high phenotypic heterogeneity and govern functions at the interface of immunity, tissue homeostasis, metabolism, and endocrine signaling. The M2 macrophages are identified based on the expression pattern of a set of M2 markers, such as transmembrane glycoproteins, scavenger receptors, enzymes, growth factors, hormones, cytokines, and cytokine receptors. Specific non-limiting examples of M2 macrophage markers include PD-L2, CD301, CD206, Arg1, Ym1/Chi3l3, and Fizzl/Relma. M2 activation is induced by fungal cells, parasites, immune complexes, complements, apoptotic cells, macrophage colony stimulating factor (MCSF), IL-4, IL-13, IL-10, tumor growth factor beta (TGF-(β), and various other signals. In addition to their role as anti-inflammatory, proresolving, wound healing, tissue repair, and trophic or regulatory, M2 macrophages can cause allergic inflammation, aid the growth of tumor tissues, and can be cellular reservoirs of various pathogens.

The terms “intestinal microbiota”, “gut flora”, and “gastrointestinal (GI) microbiota” are used interchangeably to refer to bacteria in the digestive tract.

Specific changes in GI microbiota discussed herein can be detected using various methods, including without limitation quantitative PCR or high-throughput sequencing methods which detect over- and under-represented genes in the total bacterial population (e.g., 454-sequencing for community analysis; screening of microbial 16S ribosomal RNAs (16S rRNA), etc.), or transcriptomic or proteomic studies that identify lost or gained microbial transcripts or proteins within total bacterial populations. See, e.g., U.S. Patent Publication No. 2010/0074872; Eckburg et al., Science, 2005, 308:1635-8; Costello et al., Science, 2009, 326:1694-7; Grice et al., Science, 2009, 324:1190-2; Li et al., Nature, 2010, 464: 59-65; Bjursell et al., Journal of Biological Chemistry, 2006, 281:36269-36279; Mahowald et al., PNAS, 2009, 14:5859-5864; Wikoff et al., PNAS, 2009, 10:3698-3703.

As used herein, the term “probiotic” refers to a substantially pure bacteria (i.e., a single isolate, live or killed), or a mixture of desired bacteria, or bacterially-derived products, and may also include any additional components that can be administered to a mammal. Such compositions are also referred to herein as a “bacterial inoculant.” Clostridia-containing probiotics or bacterial inoculant compositions of the invention are preferably administered with a buffering agent (e.g., to allow the bacteria to survive in the acidic environment of the stomach and to grow in the intestinal environment). Non-limiting examples of useful buffering agents include saline, sodium bicarbonate, milk, yogurt, infant formula, and other dairy products.

As used herein, the term “prebiotic” refers to an agent that increases the number and/or activity of one or more desired bacteria. Non-limiting examples of prebiotics useful in the methods and compositions of the present invention include fructooligosaccharides (e.g., oligofructose, inulin, inulin-type fructans), galactooligosaccharides, N-acetylglucosamine, N-acetylgalactosamine, glucose, other five- and six-carbon sugars (such as arabinose, maltose, lactose, sucrose, cellobiose, etc.), amino acids, alcohols, resistant starch (RS), and mixtures thereof. See, e.g., Ramirez-Farias et al., Br J Nutr (2008) 4:1-10; Pool-Zobel and Sauer, J Nutr (2007), 137:2580S-2584S.

As used herein, the term “dysbiosis” refers to a microbial imbalance on or inside the body. Dysbiosis is most commonly reported as a condition in the GI tract. It has been reported to be associated with a wide variety of illnesses, such as, e.g., irritable bowel syndrome, inflammatory bowel disease, chronic fatigue syndrome, obesity, rheumatoid arthritis, ankylosing spondylitis, bacterial vaginosis, colitis, etc. Dysbiosis can result from, e.g., antibiotic exposure as well as other causes, e.g., infections with intestinal pathogens including viruses, bacteria and eukaryotic parasites.

As used herein, the term “16S rRNA sequencing” refers to the sequencing of 16S ribosomal RNA (rRNA) gene sequences by using primers such as universal primers and/or species-specific primers to identify the bacteria present in a sample. 16S rRNA genes contain both highly conserved sites and hypervariable regions that can provide species-specific signature sequences useful for identification of bacteria. Such universal primers are well known in the art.

The terms “treat” or “treatment” of a state, disorder or condition include:
(1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or
(2) inhibiting the state, disorder or condition, i.e., arresting, reducing or delaying the development of the disease or a relapse thereof (in case of maintenance treatment) or at least one clinical or sub-clinical symptom thereof; or
(3) relieving the disease, i.e., causing regression of the state, disorder or condition or at least one of its clinical or sub-clinical symptoms.

The benefit to a subject to be treated is either statistically significant or at least perceptible to the patient or to the physician.

A “therapeutically effective amount” means the amount of a compound (e.g., a type 2 cytokine and/or a mucin) or a bacterial inoculant (e.g., bacteria of the Clostridia class) that, when administered to a subject for treating a state, disorder or condition, is sufficient to effect such treatment. The “therapeutically effective amount” will vary depending on the compound or bacteria administered as well as the disease and its severity and the age, weight, physical condition and responsiveness of the mammal to be treated.

In the methods of the present invention, therapeutically effective cytokine, mucin and helminth doses can be determined by determining the minimum dose required for the induction of M2 macrophages in the GI tract (e.g., as monitored by expression of PD-L2, CD301, CD206, Arg1, Ym1/Chi3l3, Fizz1/Relma, etc.) and/or by the inhibition of Bacteroides vulgatus abundance in the stool and/or by the expansion of Clostridial strains in the stool.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers useful in the methods and compositions of the present invention can be, e.g., sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to nanoparticles (e.g., mucoadhesive nanoparticles, negatively charged carboxylate- or sulfate-modified particles, etc.), one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc. : Hoboken, N.J.; and Enna et al. eds. (2005) Current Protocols in Pharmacology, John Wiley and Sons, Inc.: Hoboken, N.J. Additional techniques are explained, e.g., in U.S. Pat. No. 7,912,698 and U.S. Patent Appl. Pub. Nos. 2011/0202322 and 2011/0307437.

### Methods of the Invention

In one aspect, the invention provides a method for increasing the amount or activity of one or more bacterial species of the Clostridia class in the gastrointestinal tract of a subject (e.g., human) comprising administering to said subject a therapeutically effective amount of a type 2 cytokine and/or a mucin.

In another aspect, the invention provides a method for treating dysbiosis in the gastrointestinal tract of a subject (e.g., human) in need thereof, wherein the dysbiosis is associated with a decrease in the amount or activity of one or more bacterial species of the Clostridia class in the gastrointestinal tract of the subject, said method comprising administering to said subject a therapeutically effective amount of a type 2 cytokine and/or a mucin.

In a further aspect, the invention provides a method for treating a gastrointestinal or inflammatory disorder in a subject (e.g., human) in need thereof, which disorder can be treated by increasing the amount or activity of one or more bacterial species of the Clostridia class in the gastrointestinal tract of the subject, said method comprising administering to said subject a therapeutically effective amount of a type 2 cytokine and/or a mucin.

In yet another aspect, the invention provides a method for promoting a wound healing in the gastrointestinal tract of a subject (e.g., human) in need thereof comprising administering to said subject a therapeutically effective amount of a type 2 cytokine and/or a mucin.

In one embodiment of any of the above methods, the method further comprises administering to said subject bacteria of the Clostridia class. Such bacteria of the Clostridia class can be from one or more different species and can be administered, for example, as live bacterial cells, conditionally lethal bacterial strains, killed bacterial cells, spores, or bacterially-derived products. In one specific embodiment, said bacteria of the Clostridia class are administered together with (i) a carrier and/or buffering agent and/or (ii) one or more prebiotic agents which enhance growth or activity of said bacteria. Type 2 cytokine and/or a mucin and bacteria of the Clostridia class can be administered sequentially or simultaneously (in one composition or in two or more separate compositions). In one embodiment, said bacteria of the Clostridia class are from one or more human-derived commensal bacterial species belonging to Clostridium Cluster IV, XIVa, or XVIII. In one embodiment, said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense. In one embodiment, said bacteria of the Clostridia class are from one or more species selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLG055, Erysipelotrichaceae bacterium 2_2_44A, Clostridium indolis, Anaerostipes caccae, Clostridium bolteae, Lachnospiraceae bacterium DJF_VP30, Lachnospiraceae bacterium 3_1_57 FAA_CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662. In one embodiment, said bacteria of the Clostridia class are from one or more species selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLG055, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA_CT1, Clostridium sp. 316002/08, and Clostridiales bacterium 1_7_47 FAA.

In one embodiment of any of the above methods, the method further comprises administering an effective amount of one or more Helminth species (e.g., Trichuris trichiura, Schistosoma mansoni, Ancylostoma duodenale, Necator americanus, Fasciola hepatica, or Taenia saginata).

Non-limiting examples of type 2 cytokines useful in the methods of the invention include IL-13, IL-4, IL-22, IL-25, IL-33, and thymic stromal lymphopoietin (TSLP). In one embodiment, the type 2 cytokine is a fusion protein comprising, e.g., an amino acid sequence of a mature type 2 cytokine protein and CH₂ and CH₃ domains of Fc region of IgG. Non-limiting specific examples of such fusion proteins include, e.g.:

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTERSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

In one embodiment of any of the methods of the invention, two or more type 2 cytokines can be used and can be administered either sequentially or simultaneously (in one composition or in two or more separate compositions).

In one embodiment of the methods of the invention, mucin comprises one or more molecules selected from the group consisting of MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, and MUC21. In one specific embodiment, the mucin comprises MUC2.

In the methods of the invention, wherein the method comprises administering a type 2 cytokine and a mucin, they can be administered either sequentially or simultaneously (in one composition or in two or more separate compositions).

In some embodiments of the invention, the therapeutically effective amount of type 2 cytokine and/or mucin and/or helminth corresponds to the minimum dose required for the induction of M2 macrophages in the GI tract of the subject being treated (e.g., as detected by monitoring the expression of one or more of PD-L2, CD301, CD206, Arg1, Ym1/Chi3l3, and Fizz1/Relma).

In some embodiments of the invention, the therapeutically effective amount of type 2 cytokine and/or mucin and/or helminth corresponds to the minimum dose required for the decrease of Bacteroides vulgatus abundance in the stool of the subject (e.g., at least 90% decrease of Bacteroides vulgatus abundance in the stool of the subject, preferably, at least 99% decrease of Bacteroides vulgatus abundance in the stool of the subject). In some embodiments of the invention, the therapeutically effective amount of type 2 cytokine and/or mucin and/or helminth corresponds to the minimum dose required for the increase of Clostridial species abundance in the stool of the subject (e.g., at least 100% increase of said Clostridial species abundance in the stool of the subject, preferably, at least 200% increase of said Clostridial species abundance in the stool of the subject). The abundance of Bacteroides vulgates and Clostridial species can be determined, e.g., by 16S rRNA sequencing. The abundance of Clostridial species can be measured, e.g., using probes specific for one or more Clostridial strains (such as those found in Table 5, below).

Non-limiting examples of the gastrointestinal or inflammatory disorders which can be treated by the methods of the invention include autoimmune diseases, allergic diseases, infectious diseases, and rejection in organ transplantations, inflammatory bowel disease (IBD), ulcerative colitis, Crohn's disease, sprue, autoimmune arthritis, rheumatoid arthritis, Type I diabetes, multiple sclerosis, graft vs. host disease following bone marrow transplantation, osteoarthritis, juvenile chronic arthritis, Lyme arthritis, psoriatic arthritis, reactive arthritis, spondyloarthropathy, systemic lupus erythematosus, insulin dependent diabetes mellitus, thyroiditis, asthma, psoriasis, dermatitis scleroderma, atopic dermatitis, graft versus host disease, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, septic shock, toxic shock syndrome, sepsis syndrome, cachexia, acquired immunodeficiency syndrome, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrome, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondyloarthropathy, atheromatous disease/arteriosclerosis, allergic colitis, atopic allergy, food allergies such as peanut allergy, tree nut allergy, egg allergy, milk allergy, soy allergy, wheat allergy, seafood allergy, shellfish allergy, or sesame seed allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondy litis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, multiple sclerosis (all subtypes), insulindependent diabetes mellitus, sympathetic ophthalmia, pulmonary hypertension secondary to connective tissue disease, Goodpasture's syndrome, pulmonary manifestation of polyarteritis nodosa, acute rheumatio fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, allergic rhinitis (pollen allergies), anaphylaxis, pet allergies, latex allergies, drug allergies, allergic rhinoconjuctivitis, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis, diarrhea, colon cancer, cystic fibrosis, celiac disease, Type 2 diabetes, and autism-related immunopathologies.

It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

As a non-limiting example, the invention can be combined with other therapies that block inflammation (e.g., via blockage of IL1 INFα/β, IL6, TNF, IL23, etc.).

The methods of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The methods of the invention can be also combined with other treatments that possess the ability to modulate NKT function or stability, including but not limited to CD1d, CD16d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e).

### Compositions of the Invention

In conjunction with the above-identified methods of the invention, the invention provides various compositions, including, among others, compositions comprising two or more components selected from the group consisting of (i) a type 2 cytokine (e.g., one or more of IL-13, IL-4, IL-22, IL-25, IL-33, or thymic stromal lymphopoietin (TSLP)), (ii) a mucin (e.g., one or more of MUC1, MUC2, MUC3A, MUC3B, MUC4, MUCSAC, MUCSB, MUC6, MUC7, MUC8, MUC12, MUC13, MUC15, MUC16, MUC17, MUC19, MUC20, and MUC21), (iii) bacteria of the Clostridia class, (iv) bacteria (e.g., Lactobacillus), yeast or virus expressing a type 2 cytokine, (v) yeast or virus expressing a mucin, and (vi) a helminth (e.g., Trichuris trichiura, Schistosoma mansoni, Ancylostoma duodenale, Necator americanus, Fasciola hepatica, or Taenia saginata).

Bacteria of the Clostridia class can be from one or more different species and can be, e.g., in the form of live bacterial cells, conditionally lethal bacterial strains, killed bacterial cells, spores, or bacterially-derived products. In one embodiment, said bacteria of the Clostridia class are from one or more human-derived commensal bacterial species belonging to Clostridium Cluster IV, XIVa, or XVIII. In one embodiment, said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense. In one embodiment, said bacteria of the Clostridia class are from one or more species selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLGO55, Erysipelotrichaceae bacterium 2_2_44A, Clostridium indolis, Anaerostipes caccae, Clostridium bolteae, Lachnospiraceae bacterium DJF_VP30, Lachnospiraceae bacterium 3_1_57 FAA_CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662. In one embodiment, said bacteria of the Clostridia class are from one or more species selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLG055, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1—57 FAA_CT1, Clostridium sp. 316002/08, and Clostridiales bacterium 1_7_47 FAA.

Non-limiting examples of type 2 cytokines useful in the compositions of the invention include IL-13, IL-4, IL-22, IL-25, IL-33, and thymic stromal lymphopoietin (TSLP). Non-limiting examples of cytokine amino acid sequences are as follows:
Human IL-13: NP 002179 mature peptide is 35-132aa, signal peptide 1-34aa;
Mouse IL-13: NP 032381 mature peptide is 22-131aa, signal peptide 1-21aa;
Human IL-4: NP 000580 mature peptide is 25-149aa, signal peptide 1-24aa;
Mouse IL-4: NP 067258 mature peptide is 21-140aa, signal peptide 1-20aa;
Human IL-22: NP 065386 mature peptide is 34-179aa, signal peptide 1-33aa;
Mouse IL-22: XP 006513928;
Human IL-25: NP 073626 mature peptide is 33-177aa, signal peptide 1-32aa;
Mouse IL-25: NP 542767;
Human IL-33: NP 001300973 XP 005251684;
Mouse IL-33: NP 001158196;
Human TSLP: NP 149024 mature peptide is 29-159aa, signal peptide 1-28aa;
Mouse TSLP: NP 067342 mature peptide is 20-140aa, signal peptide 1-19aa.

Compositions of the invention can comprise one type 2 cytokine or two or more different type 2 cytokines. In some embodiments of the present invention, the type 2 cytokine may be expressed as a fusion protein with a portion of the Fc region of IgG. A linker region of one or more amino acids may be used to join the IgG heavy chain region to the type 2 cytokine. This linker region may contain suitable amino acids so as to provide flexibility to the linker region.

In one embodiment, the type 2 cytokine useful in the compositions of the present invention is a fusion protein comprising, e.g., an amino acid sequence of a mature type 2 cytokine protein and CH₂ and CH₃ domains of the Fc region of IgG. Non-limiting specific examples of such fusion proteins include, e.g.:

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3)
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTFRSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

In one embodiment, compositions of the invention comprise type 2 cytokine and/or mucin associated with nanoparticles.

In some embodiments of the present invention, type 2 cytokines or mucins may be expressed by genetically engineered non-invasive and non-pathogenic bacteria (e.g., non-commensal and/or non-colonizing bacteria), yeast, and/or other non-invasive, non-pathogenic, genetically tractable, easily manipulated microorganisms, and viruses, which include those that infect eukaryotic cells or bacterial cells. The microorganisms may comprise one or more nucleic acid constructs in which the nucleic acid encoding the cytokine is under control of appropriate regulatory sequences for expression (promoter, terminator, and/or enhancer). Vectors also normally contain marker genes and other sequences as appropriate.

The expression nucleic acid constructs comprising a coding sequence for a type 2 cytokine and/or mucin wherein the coding sequence is under the control of a promoter for expression in a non-invasive and/or non-pathogenic microorganism, is provided as a further aspect of the present invention. The promoter employed in accordance with the described vector can be, e.g., constitutive or inducible. The expression nucleic acid constructs encoding the type 2 cytokine and/or mucin can comprise a secretory signal sequence. Non-limiting examples of suitable secretory signal sequences include any of those with activity in Bacillus, Clostridium and/or Lactobacillus, such as, e.g., a-amylase secretion leader of Bacillus amyloliquefaciens or the secretion leader of the Staphylokinase enzyme secreted by some strains of Staphylococcus, which is known to function in both Gram-positive and Gram-negative hosts (see “Gene Expression Using Bacillus”, Rapoport (1990) Current Opinion in Biotechnology 1:21-27), or leader sequences from numerous other Bacillus enzymes or S-layer proteins (see pp 341-344 of Harwood and Cutting, “Molecular Biological Methods for Bacillus”, John Wiley & Co. 1990). To generate a recombinant microorganism for use in the present invention, nucleic acid is introduced into a host cell.

The compositions of the invention can further comprise (i) a carrier and/or buffering agent and/or (ii) one or more prebiotic agents which enhance growth or activity of one or more bacteria present in the composition. The precise nature of the carrier or other material may depend on the route of administration. For intravenous, cutaneous or subcutaneous injection, or injection at the site of an affliction, a parenterally acceptable aqueous solution may be employed which is pyrogen-free and has suitable pH, isotonicity and stability. Those of relevant skill in the art are well able to prepare suitable solutions. Preservatives, stabilizers, buffers, antioxidants and/or other additives may be included, as required.

The compositions of the invention can be formulated for various routes of administration, including, e.g., oral, rectal, fecal (e.g., by enema), and via naso/oro-gastric gavage.

### Administration and Delivery

Administration of the compounds, organisms and compositions in the methods of the invention can be accomplished by any method known in the art. Non-limiting examples of useful routes of delivery include oral, rectal, fecal (e.g., by enema), and via naso/oro-gastric gavage, as well as parenteral, intraperitoneal, intradermal, transdermal, intrathecal, nasal, and intracheal admnistration. The active agent may be systemic after administration or may be localized by the use of regional administration, intramural administration, or use of an implant that acts to retain the active dose at the site of implantation. Bacteria can be mixed with a carrier and (for easier delivery to the digestive tract) applied to liquid or solid food or feed or to drinking water. The carrier material should be non-toxic to the bacteria and the subject/patient. Non-limiting examples of bacteria-containing formulations useful in the methods of the present invention include oral capsules and saline suspensions for use in feeding tubes, transmission via nasogastric tube, or enema. If live bacteria are used, the carrier should preferably contain an ingredient that promotes viability of the bacteria during storage. The formulation can include added ingredients to improve palatability, improve shelf-life, impart nutritional benefits, and the like. If a reproducible and measured dose is desired, the bacteria can be administered by a rumen cannula. In certain embodiments, the bacteria-containing formulation used in the methods of the invention further comprises a buffering agent. Examples of useful buffering agents include saline, sodium bicarbonate, milk, yogurt, infant formula, and other dairy products.

The useful dosages of the compounds and bacteria-containing formulations of the invention will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve colonization of the GI tract with the desired bacterial inoculant, e.g. 10⁶, 10⁷, 10⁸, 10⁹, and 10¹⁰ CFU for example, can be administered in a single dose. Lower doses can also be effective, e.g., 10⁴, and 10⁵ CFU. The bacteria-containing formulation may also comprise one or more prebiotics which promote growth and/or immunomodulatory activity of the bacteria in the formulation. While it is possible to use a bacterial inoculant or compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice. Although there are no physical limitations to delivery of the formulations of the present invention, oral delivery is preferred for delivery to the digestive tract because of its ease and convenience, and because oral formulations readily accommodate additional mixtures, such as milk, yogurt, and infant formula.

Oral delivery may also include the use of nanoparticles that can be targeted, e.g., to the GI tract of the subject, such as those described in Yun et al., Adv Drug Deliv Rev. 2013, 65(6):822-832 (e.g., mucoadhesive nanoparticles, negatively charged carboxylate- or sulfate-modified particles, etc.). Non-limiting examples of other methods of targeting delivery of compositions to the GI tract are discussed in U.S. Pat. Appl. Pub. No. 2013/0149339 and references cited therein (e.g., pH sensitive compositions [such as, e.g., enteric polymers which release their contents when the pH becomes alkaline after the enteric polymers pass through the stomach], compositions for delaying the release [e.g., compositions which use hydrogel as a shell or a material which coats the active substance with, e.g., in vivo degradable polymers, gradually hydrolyzable polymers, gradually water-soluble polymers, and/or enzyme degradable polymers], bioadhesive compositions which specifically adhere to the colonic mucosal membrane, compositions into which a protease inhibitor is incorporated, a carrier system being specifically decomposed by an enzyme present in the colon).

For oral administration, the active ingredient(s) can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

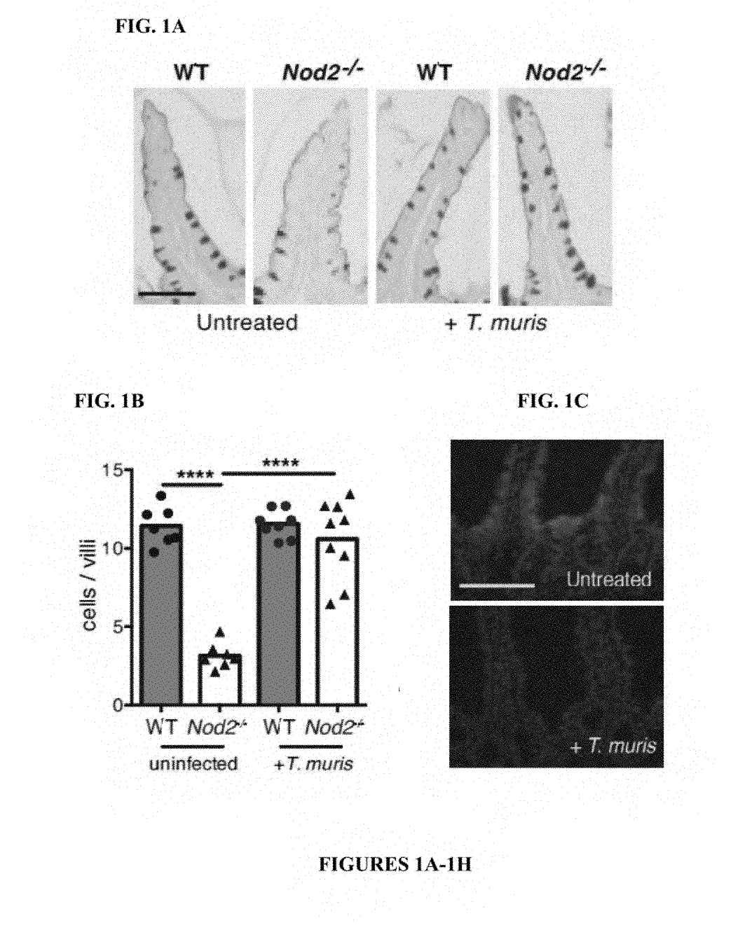

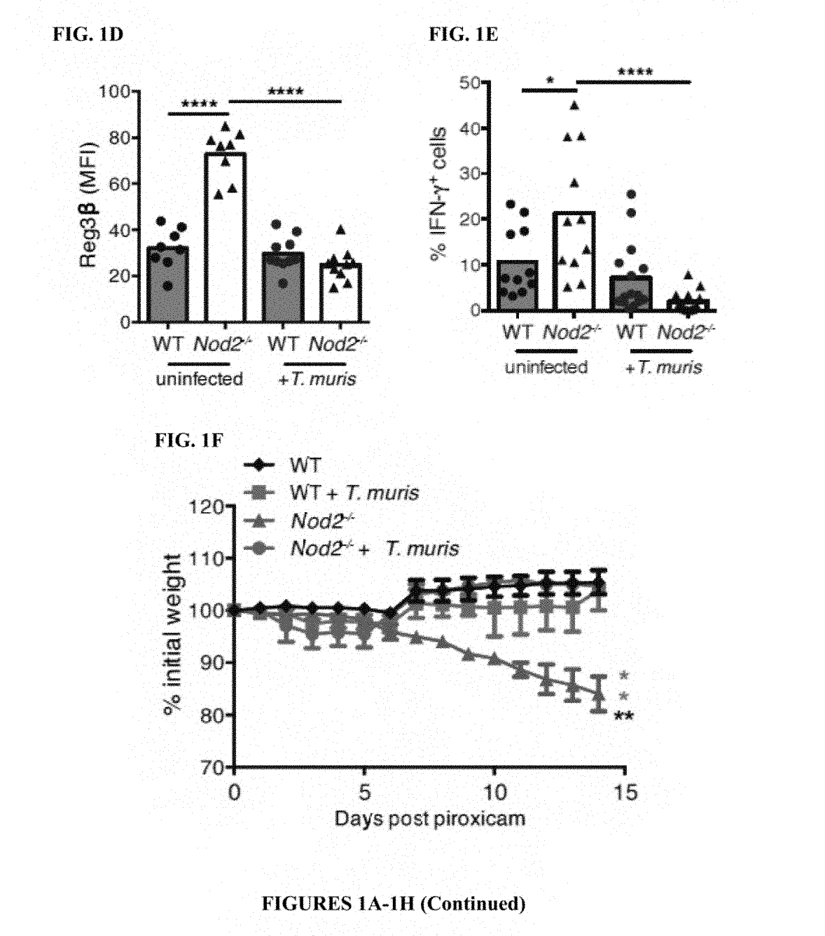

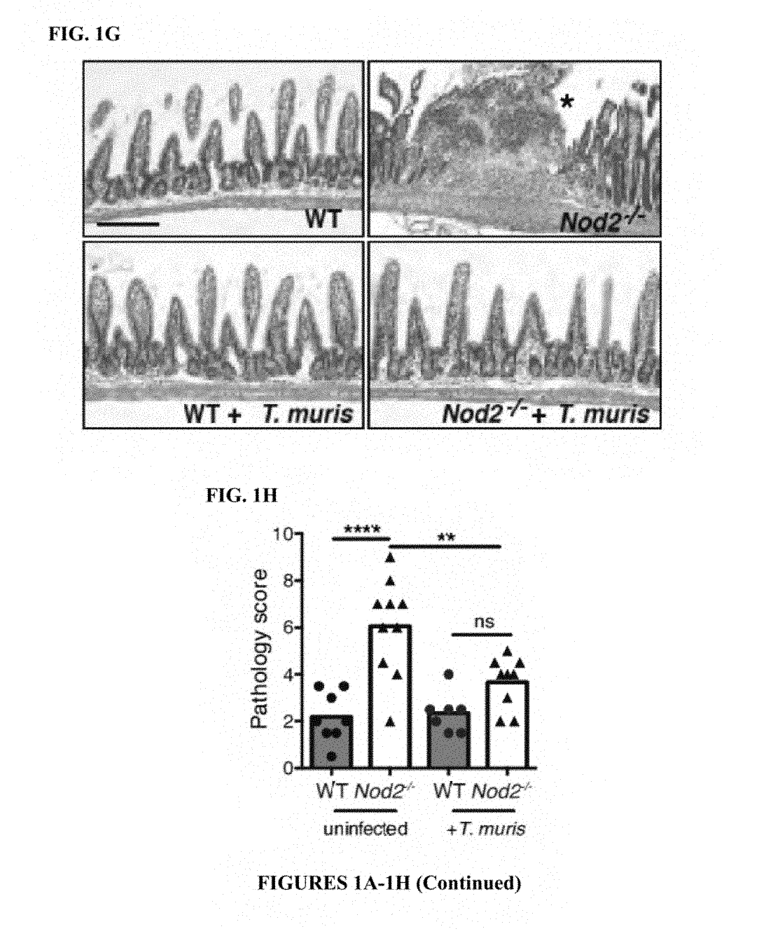

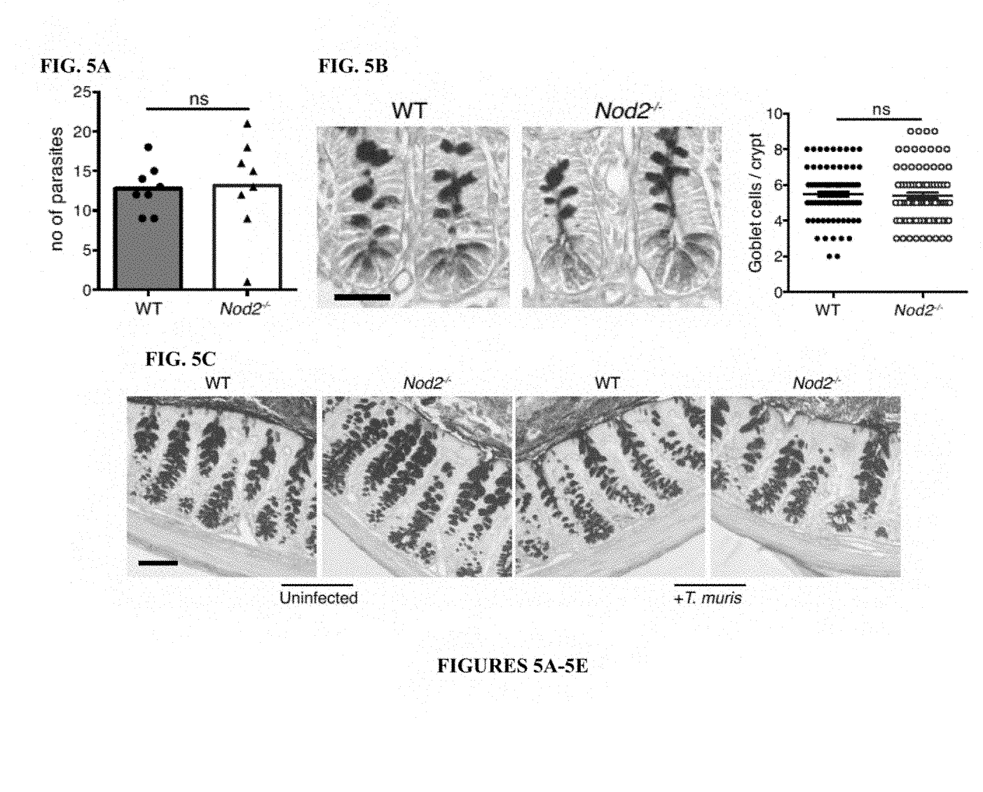

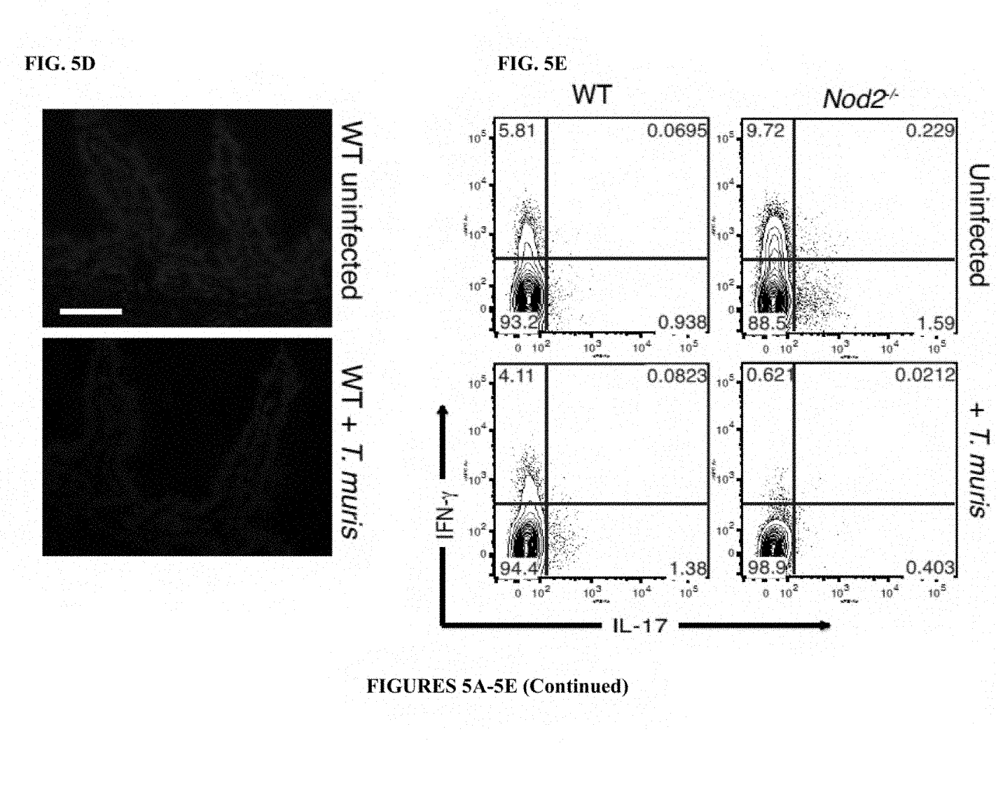

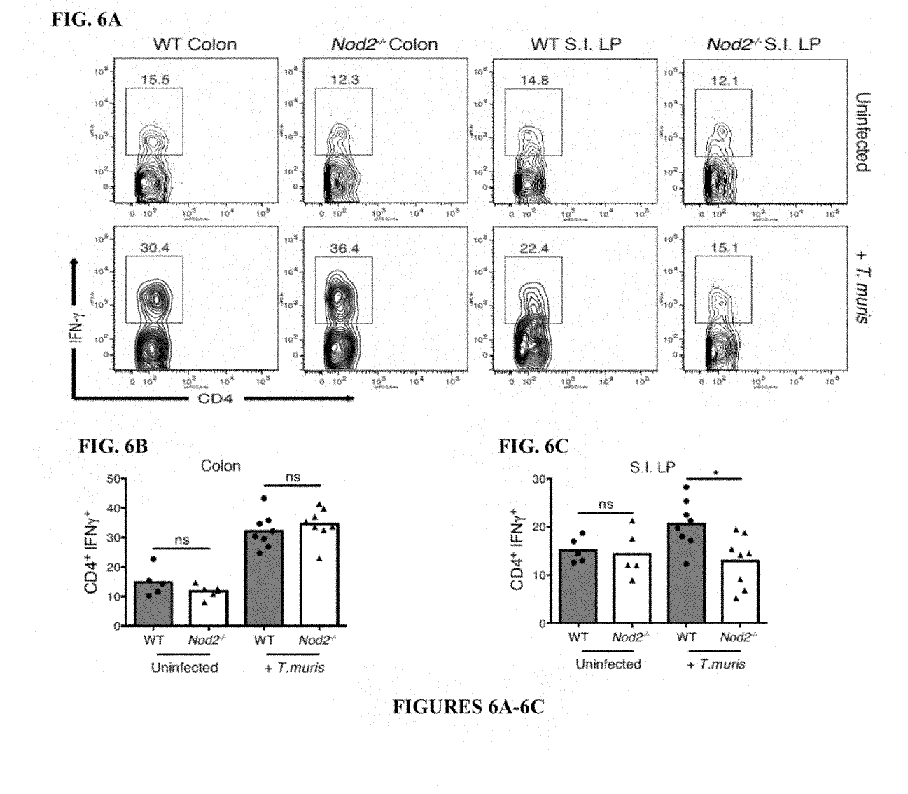

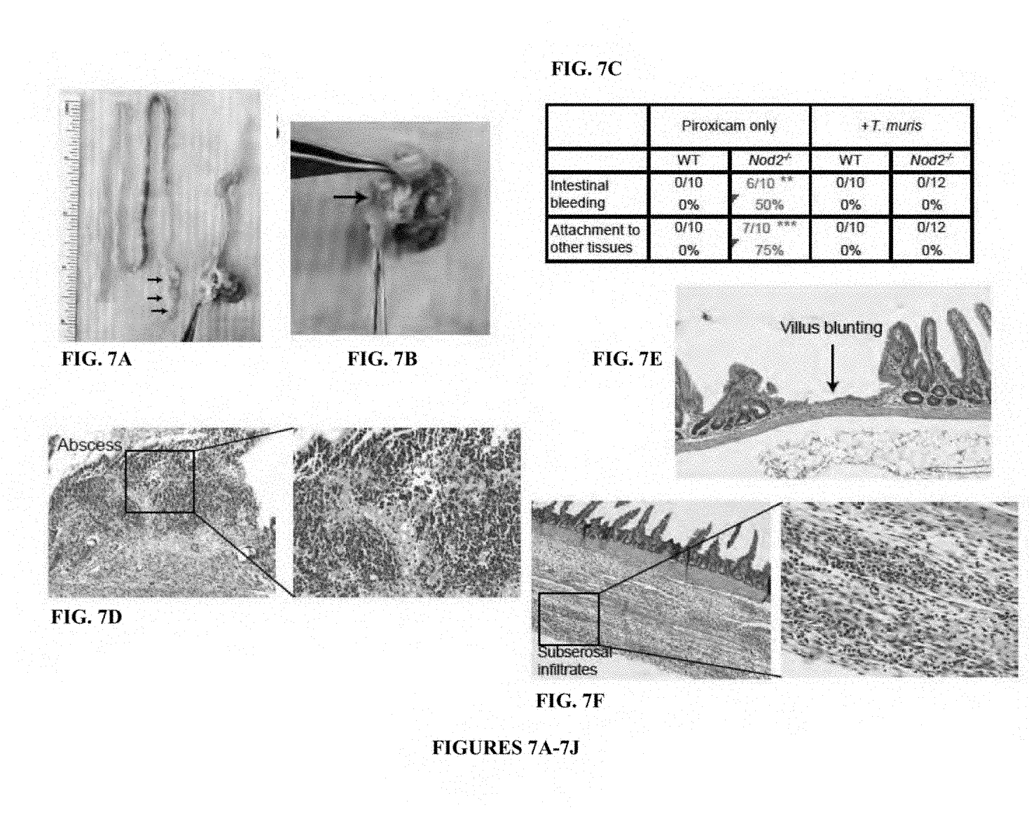

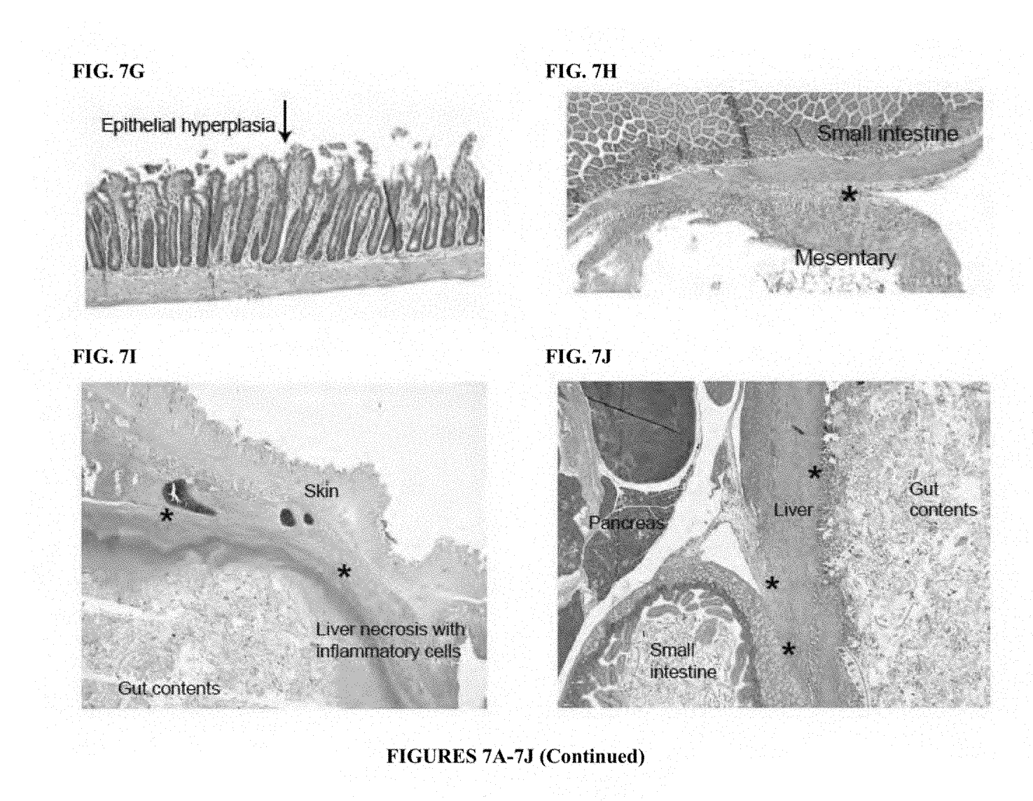

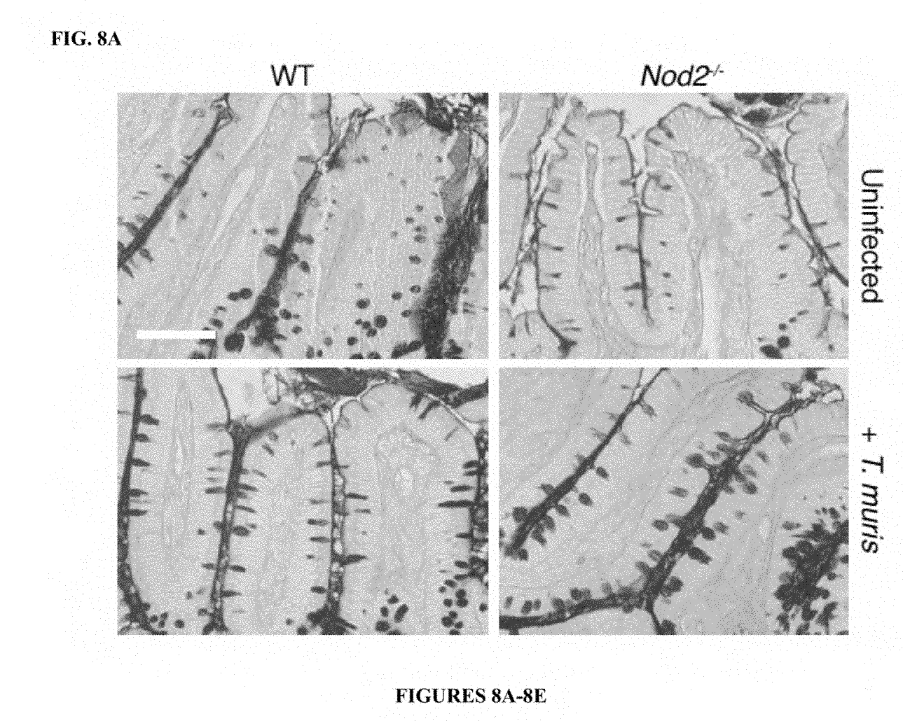

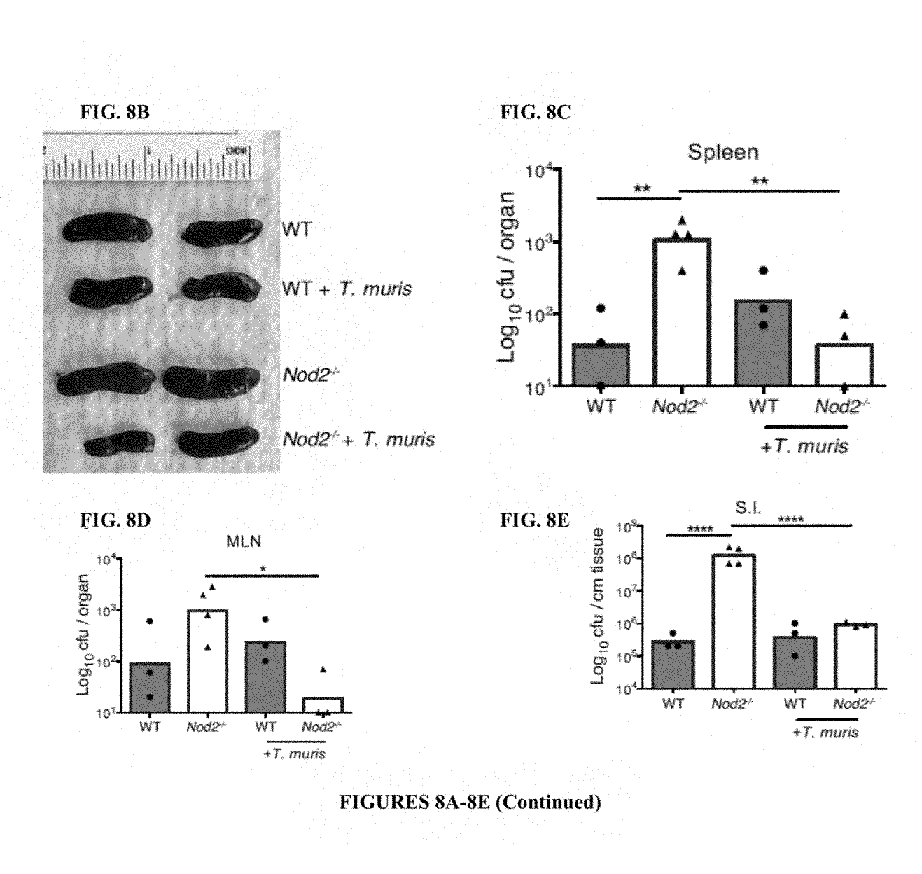

The inventors previously found that mice deficient in Nod2 develop several small intestinal (SI) abnormalities in a manner dependent on a ubiquitous member of the gut microbiota, Bacteroides vulgatus (6). Consistent with the specific association between NOD2 variants and ileal Crohn's disease (CD) (7), an IBD that affects the SI, the most striking abnormality was a SI goblet cell defect that resulted in a compromised mucus layer, allowing sustained colonization by B. vulgatus. The inventors also found that chronic infection of Nod2^{-/-} mice with the parasitic worm Trichuris muris restored SI goblet cell numbers and morphology (FIGS. 1A-1B, 5A-5B). These changes were not detected in the colon, and wild-type (WT) mice infected with T. muris did not display non-specific goblet cell hyperplasia (FIG. 5C). Elevated epithelial levels of the antimicrobial lectin Reg3β and interferon (IFN)γ+ CD830 intraepithelial lymphocytes (IELs), inflammatory markers associated with goblet cell defects in Nod2^{-/-} mice (6), were also reduced upon T. muris infection (FIGS. 1C-1E, 5D-5E, 6). Nod2^{-/-} mice develop severe intestinal pathologies following SI injury induced by the non-steroidal anti-inflammatory drug (NSAID) piroxicam. T. muris infection prevented the intestinal bleeding and perforation, exaggerated weight loss, mucus depletion, splenomegaly, and bacterial translocation that were observed in uninfected Nod2^{-/-} mice treated with piroxicam (FIGS. 1F, 7A-7C, 8). Blind histology analysis confirmed reductions in specific pathologies such as abscesses, epithelial hyperplasia, villus blunting, and immune infiltrates (FIGS. 1G-1H, 7D-7J). These results indicate that T. muris infection ameliorates spontaneous and inducible intestinal defects in Nod2^{-/-} mice.

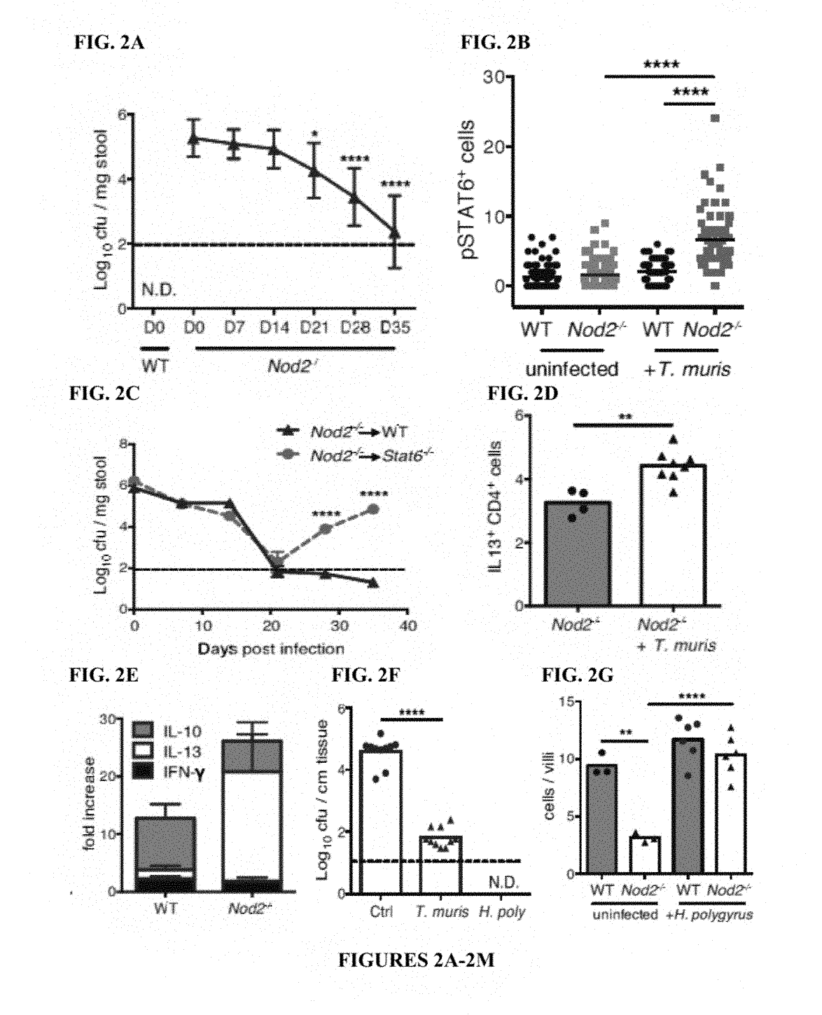

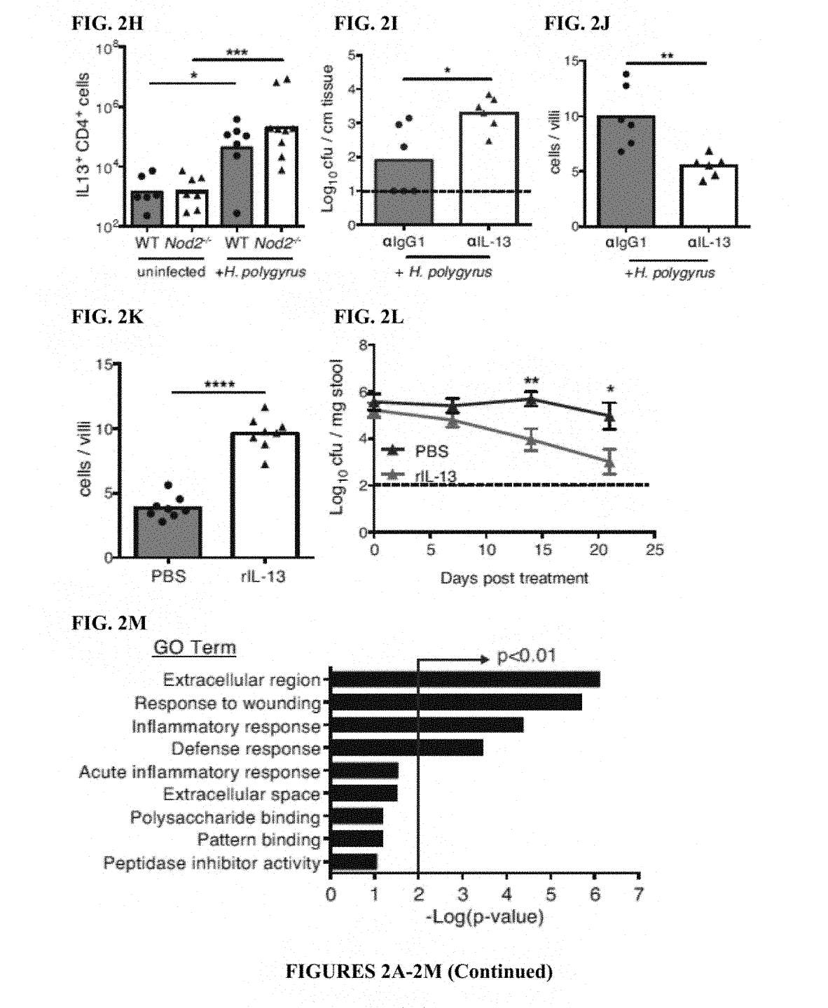

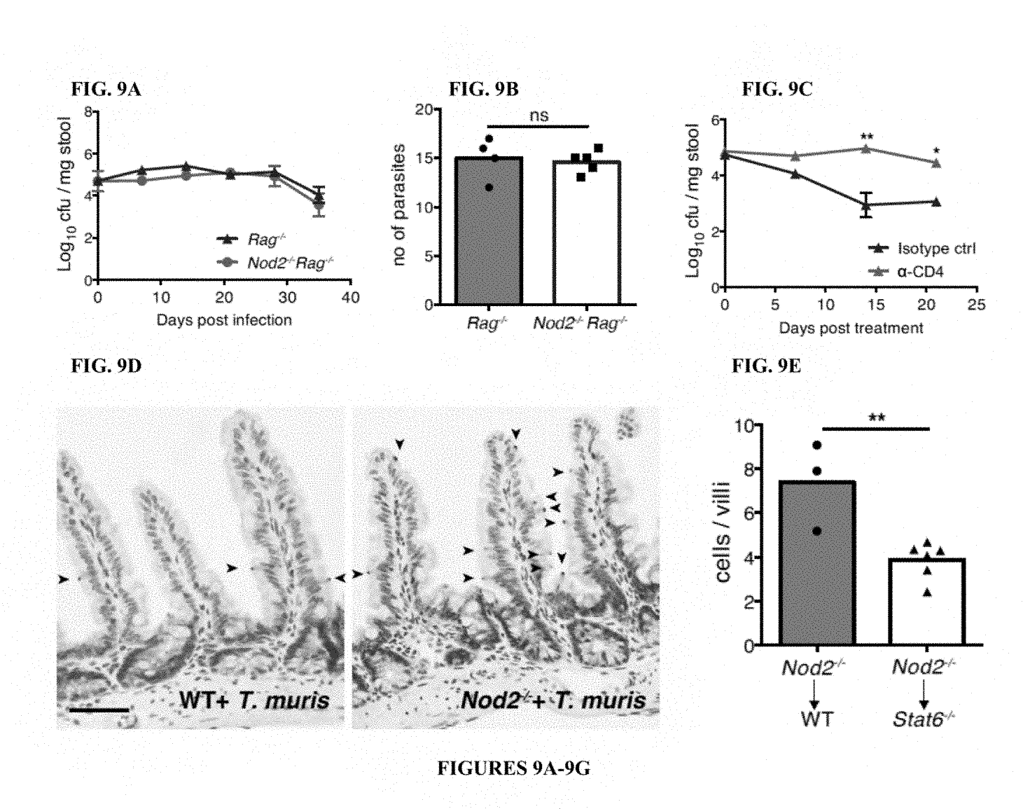

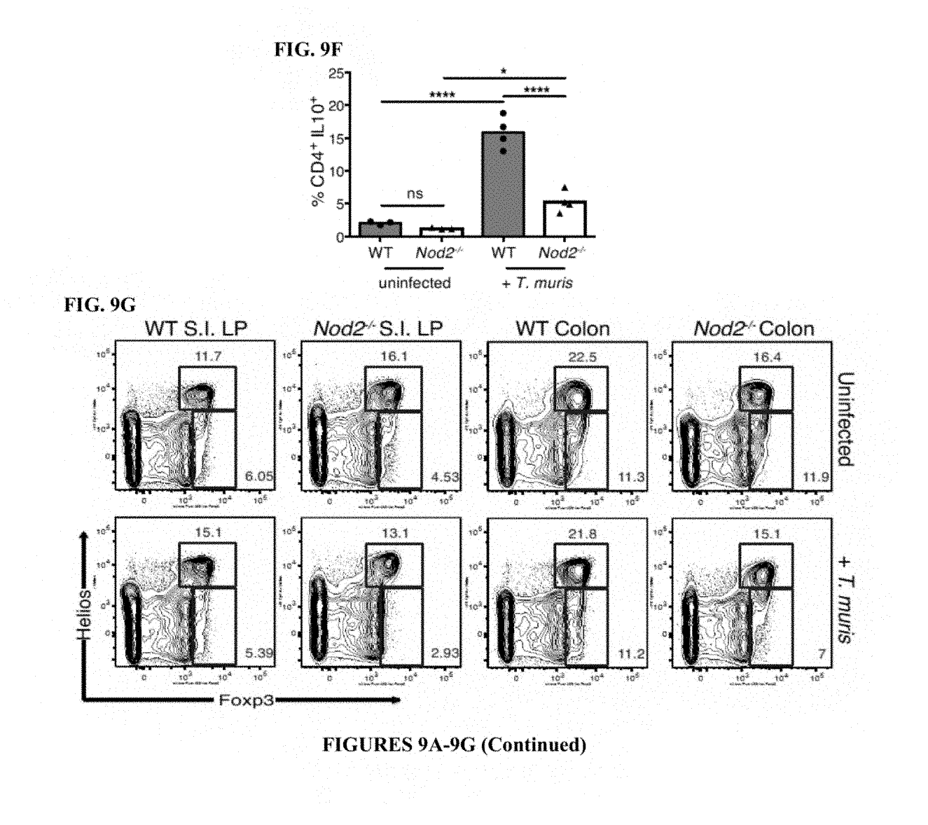

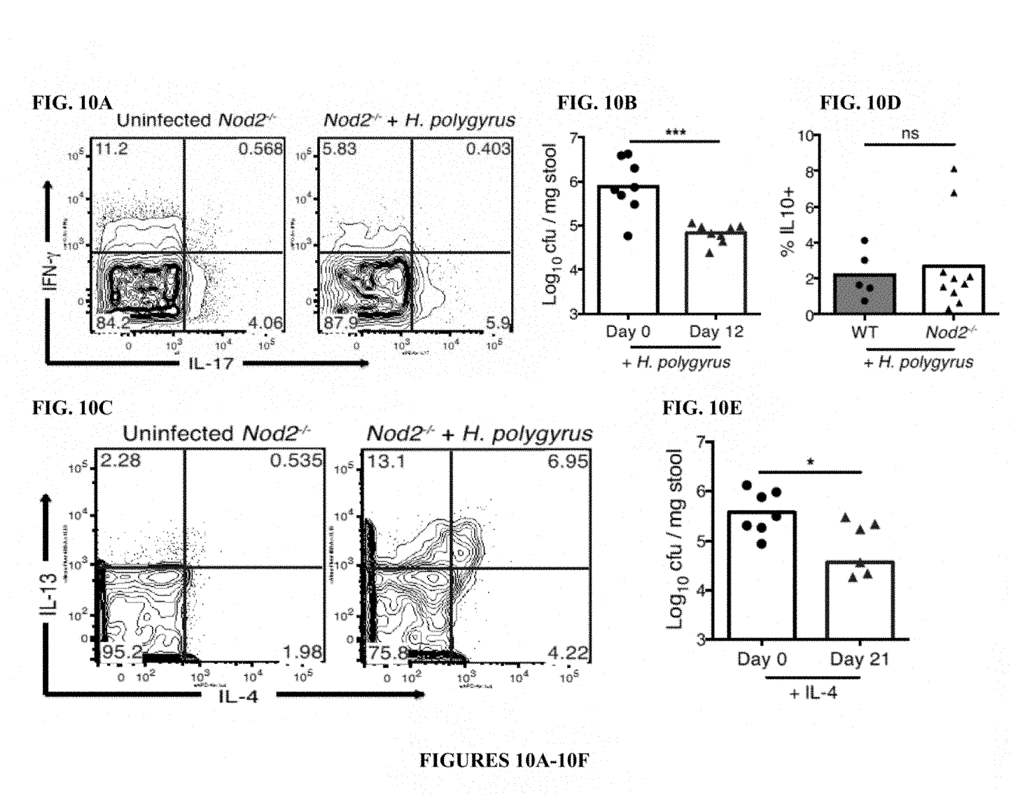

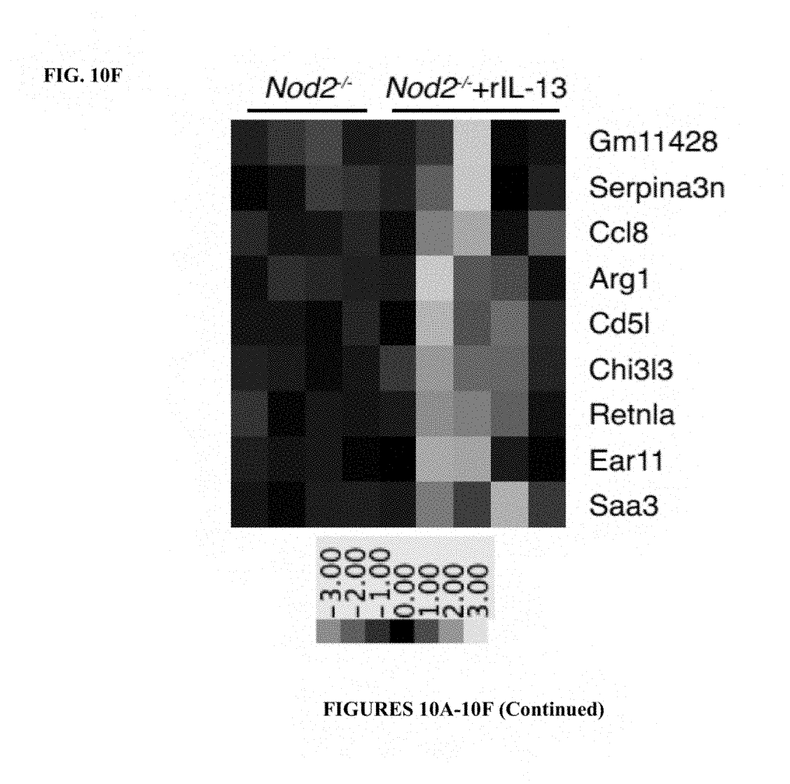

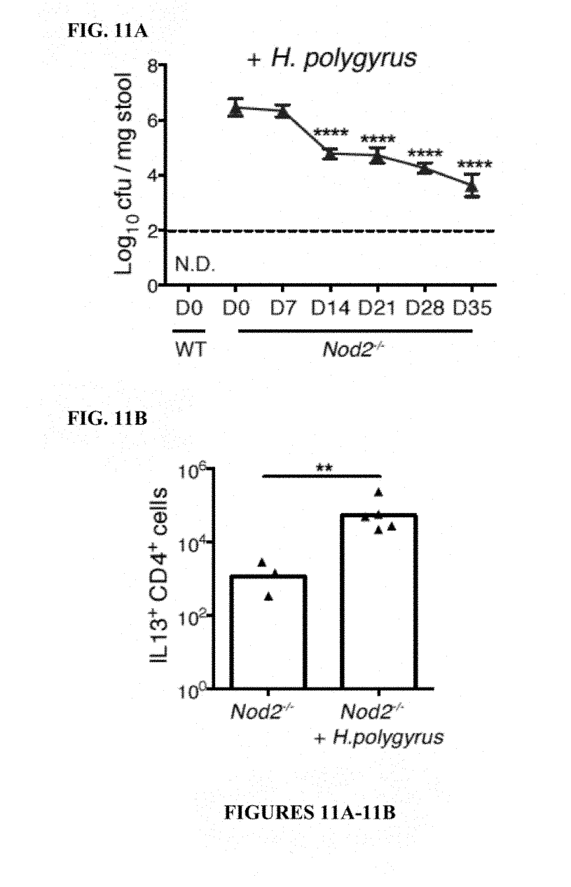

Consistent with the dependence of these inflammatory pathologies on B. vulgatus (6), T. muris infection reduced bacterial burden to the limit of detection in the stool and SI tissue of Nod2^{-/-} mice (FIG. 2A, 2F). B. vulgatus inhibition was dependent on lymphocytes (FIG. S5A-C), potentially reflecting goblet cell activation by type-2 cytokines (interleukin (IL)-4 and IL-13) produced by T helper (T_{H}) cells during helminth infections. Indeed, increased phosphorylation of the type-2 transcription factor Stat6 was found in the SI epithelium of T. muris-infected Nod2^{-/-} mice (FIGS. 2B, 9D). Also, T. muris infection only transiently inhibited B. vulgatus and did not restore goblet cells in State^{-/-} mice reconstituted with Nod2^{-/-} bone marrow (FIGS. 2C, 9E). T. muris-infected Nod2^{-/-} mice displayed a dominant T_{H}2 response characterized by a >10-fold increase in IL-13+ CD4+ T cells in the lamina propria (FIGS. 2D-2E, 9F-9G). These results were confirmed with a second helminth, Heligmosomoides polygyrus, which induced an even greater T_{H}2 response compared with T. muris, perhaps reflecting the distinct anatomical niches of these parasites (FIGS. 2H, 2D; 10C, 10D; 11B). H. polygyrus completely abolished tissue-associated B. vulgatus, restored goblet cells, and reduced IFNγ+ IELs in Nod2^{-/-} mice (FIGS. 2F, 2G; 10A, 10B, 11A). Blocking IL-13 inhibited the effect of H. polygyrus on B. vulgatus and goblet cells, and administering recombinant IL-13 (rIL-13) or rIL-4 to Nod2^{-/-} mice was sufficient to reproduce the effect of helminth infection (FIGS. 2I, 2J, 2K, 2L; 10E). RNA-seq analysis of intestinal tissues from rIL-13 treated Nod2^{-/-} mice revealed a wound healing response characterized by expression of M2 macrophage genes (FIGS. 2M, 10F, Table 1). These results are consistent with the anti-inflammatory role of M2 macrophages in the gut (8, 9), and help explain how helminth infection ameliorates the exacerbated intestinal injury response in Nod2^{-/-} mice. These results do not contradict the regulatory response induced by H. polygyrus in the colon (9, 10), because type-2 immunity and regulatory T cells can function concurrently to reduce inflammation (11).

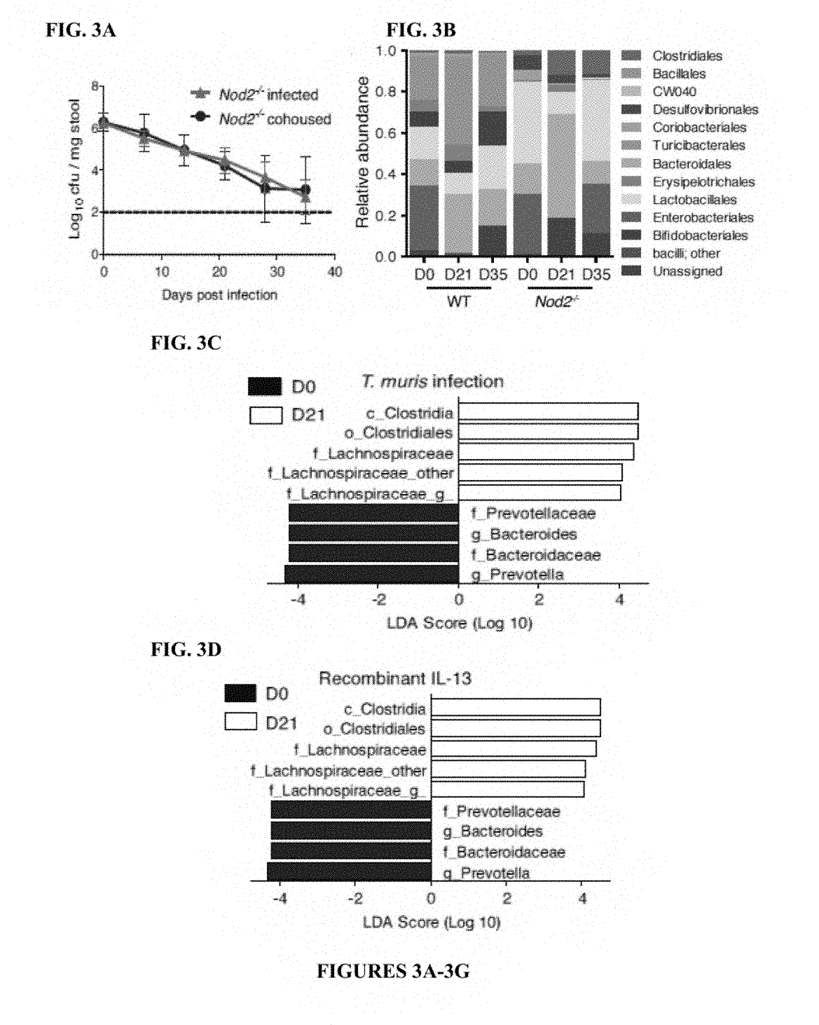

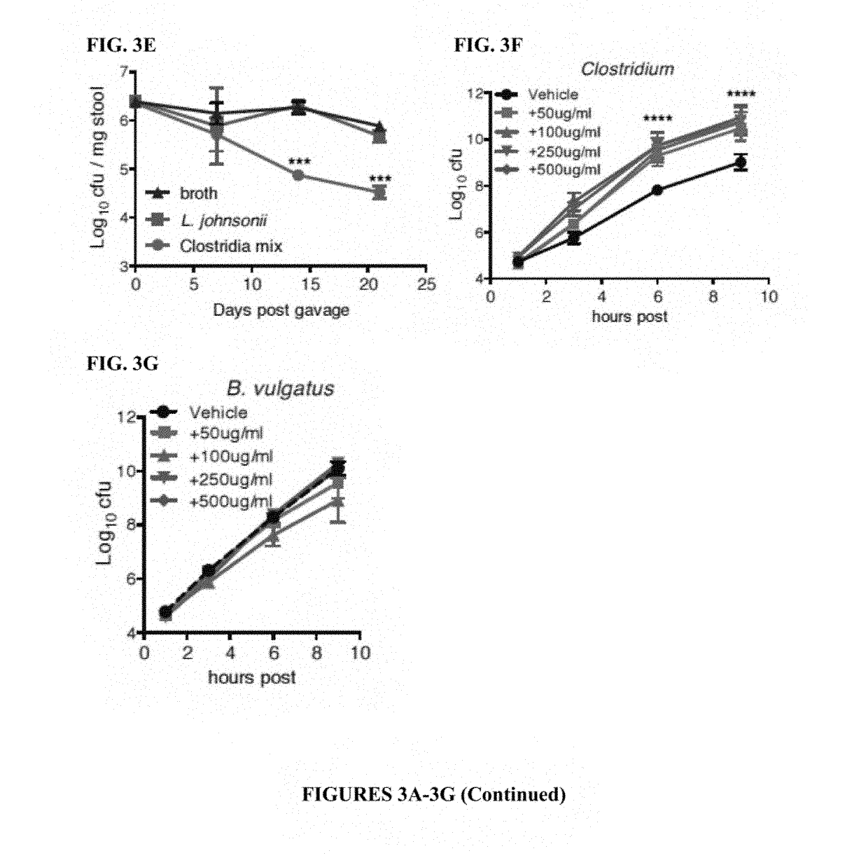

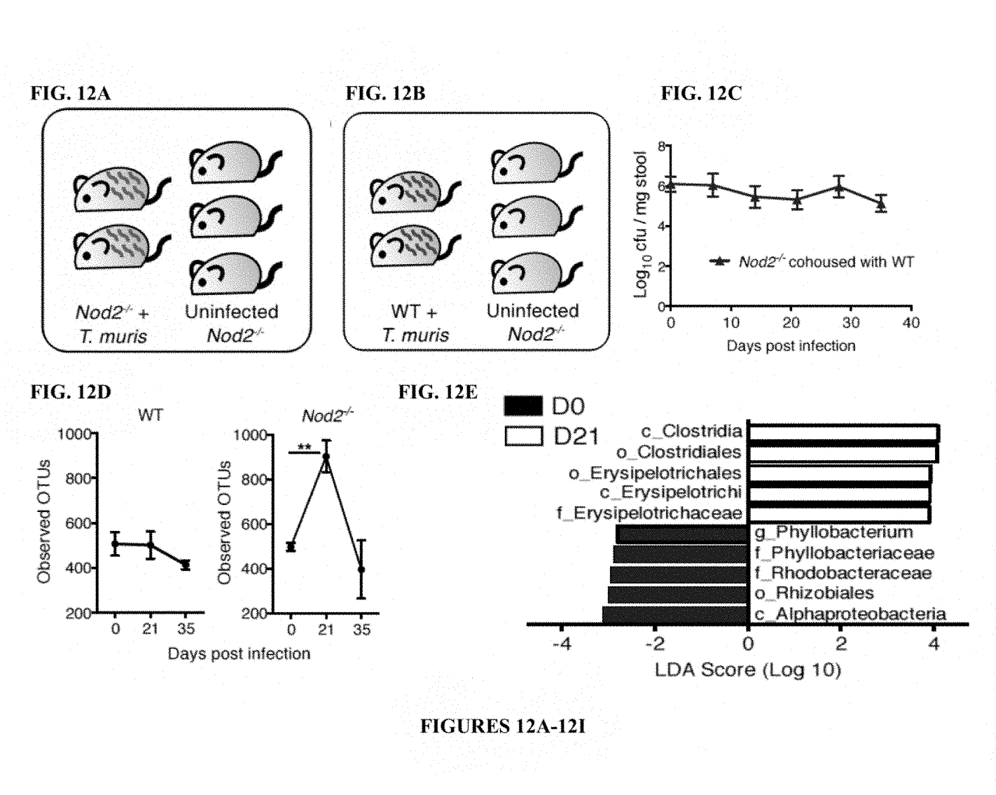

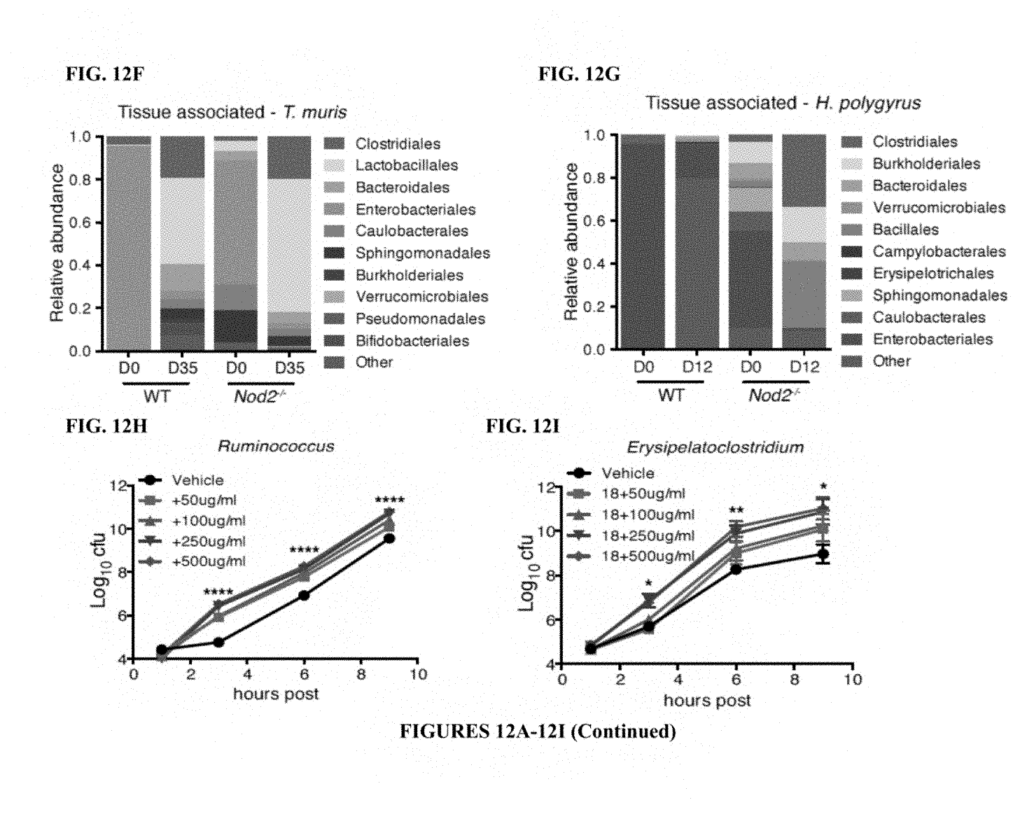

The reduction of B. vulgatus in the presence of helminths could be mediated indirectly through alterations to the gut microbiota downstream of the type-2 response. Cohousing mice allows for coprophagic transmission of microbial populations without transfer of parasites because the worms are not sexually mature until ˜35 days post infection and eggs require several weeks for germination (12). The inventors found that uninfected Nod2^{-/-} mice cohoused with T. muris-infected Nod2^{-/-} mice showed a similar decrease in B. vulgatus colonization (FIGS. 3A, 12A). This reduction in B. vulgatus levels was not observed in uninfected Nod2^{-/-} mice when they were instead cohoused with T. muris-infected WT mice (FIG. 12B, 12C). 16S rDNA sequencing analysis of stool samples indicated that the alterations to microbial community compositions are different for T. muris-infected WT and Nod2^{-/-} mice (FIG. 3B), which may reflect different intestinal responses between WT and Nod2^{-/-} mice (FIG. 2E). Whereas there is reduced alpha diversity in infected WT mice, as previously reported (13, 14), Nod2^{-/-} mice increased their alpha diversity at Day 21 post infection (FIG. 12D). The most significantly reduced bacterial taxa in infected Nod2^{-/-} mice were Prevotella and Bacteroides genus (belonging to the order Bacteroidales), and the Lachnospiraceae family of the order Clostridiales were the most significantly increased (FIG. 3C). The increase in Clostridiales was less evident in WT mice (FIG. 3B), potentially explaining why cohousing Nod2^{-/-} mice with T. muris-infected WT mice was ineffective in reducing B. vulgatus burden. The expansion of Clostridiales was also observed in the stool of uninfected Nod2^{-/-} mice treated with rIL-13 or rIL-4 (FIGS. 3D, 12E). The expansion of Clostridiales was even more pronounced among tissue-associated bacteria in the SI following T. muris or H. polygyrus infection (FIG. 12F-12G). Thus, helminth infection and type-2 cytokines inhibit B. vulgatus and expand Clostridiales strains.

To determine if Clostridia can directly inhibit B. vulgatus, the inventors inoculated Nod2^{-/-} mice with a mixture of clusters IV, XIVa, and XVIII Clostridiales and Erysipelotrichales strains isolated from human feces (15). Repetitive gavaging of Nod2^{-/-} mice with this mixture, but not sterile broth or an equivalent number of Lactobacillus johnsonii (a host-interactive commensal bacterium (16)), led to a decrease in B. vulgatus over time (FIG. 3E). Increased mucus production by goblet cells may alter the intestinal environment to favor Clostridiales, because the addition of mucin to anaerobic cultures was demonstrated to accelerate the growth of all three representative Clostridia strains tested but not B. vulgatus (FIGS. 3F-3G; 12H, 12I). Hence, these results suggest that in Nod2^{-/-} mice, the mucus response associated with type-2 immunity during helminth infection expands Clostridia strains that can inhibit colonization of B. vulgatus.

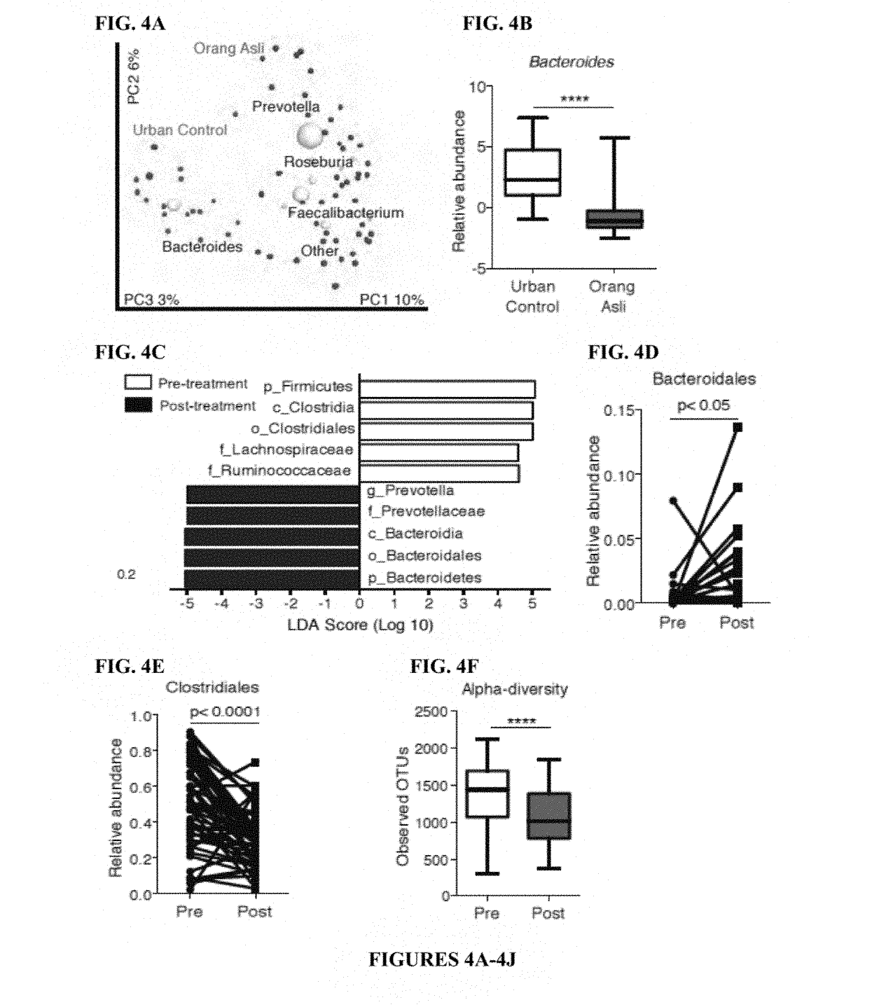

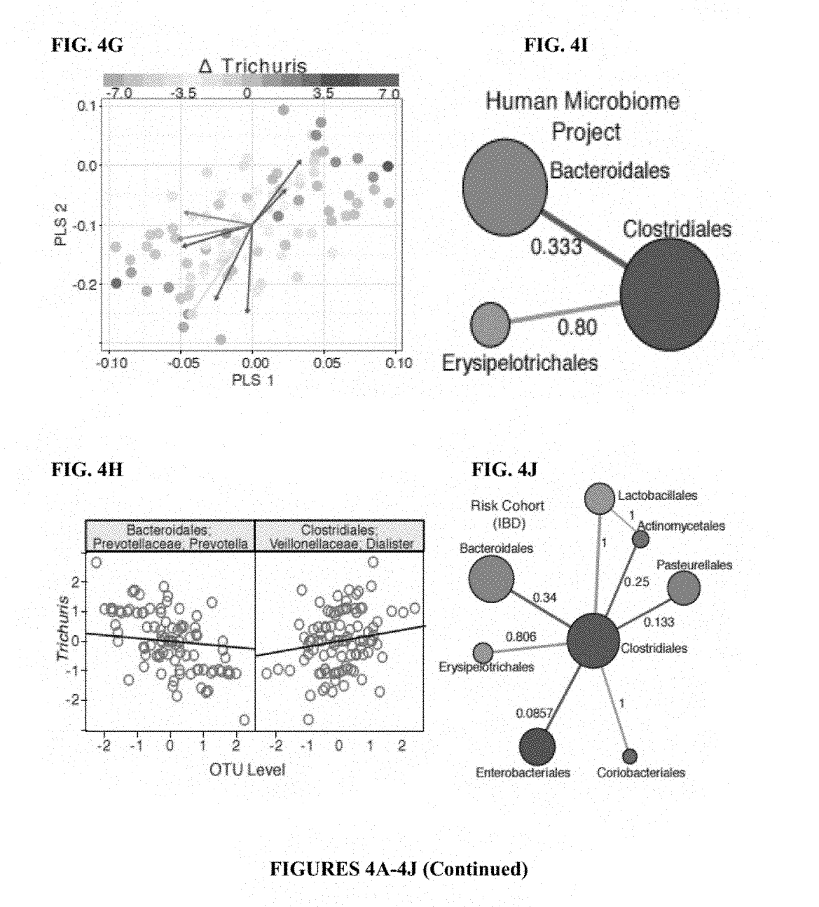

IBD is less prevalent in regions where helminth colonization is endemic. The inventors previously found that helminth-colonized individuals among indigenous populations in Malaysia, known as the Orang Asli, have higher microbial diversity than negative individuals (17). Rural Orang Asli of the Temuan subtribe from a village 40 km away were compared with individuals living in urbanized Kuala Lumpur (96% versus 5.3% of individuals colonized by intestinal helminths, respectively) (Table 2). People living in Kuala Lumpur predominantly cluster in a group driven by abundance of a single Bacteroides OTU (TaxID 3600504), which is less abundant in the Orang Asli (FIG. 4A, 4B). In contrast, the helminth-positive Orang Asli falls into a second group characterized by Faecalibacterium and Prevotella (FIG. 4A). This division between urban and rural populations in microbiota dominances is observed in other Asian countries (18).

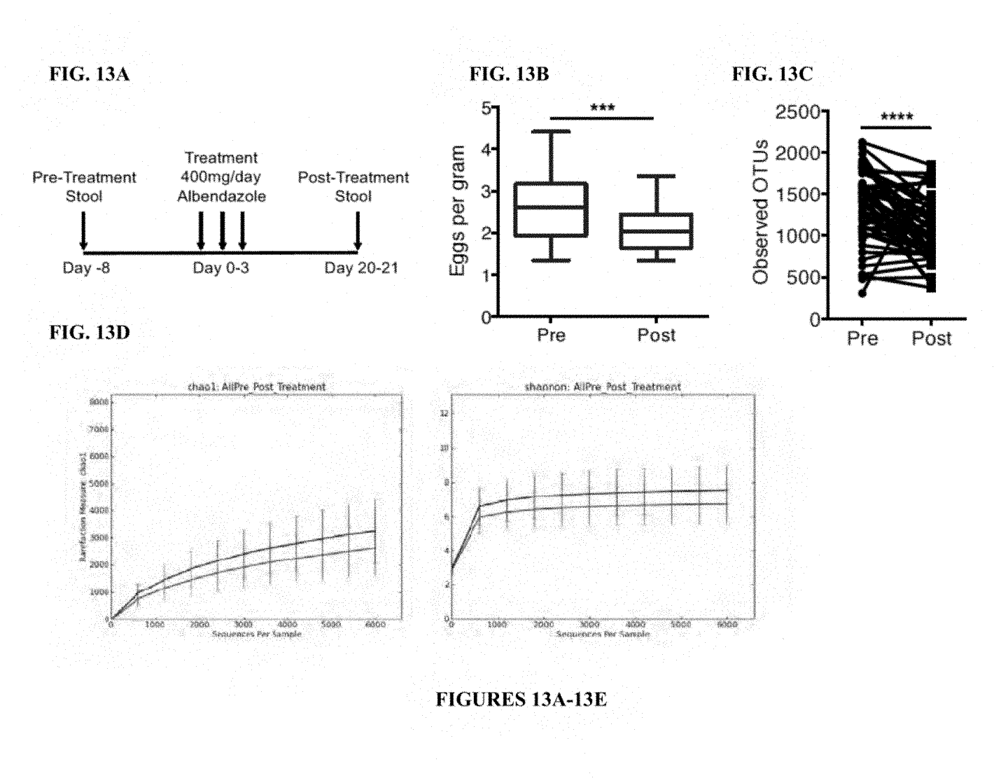

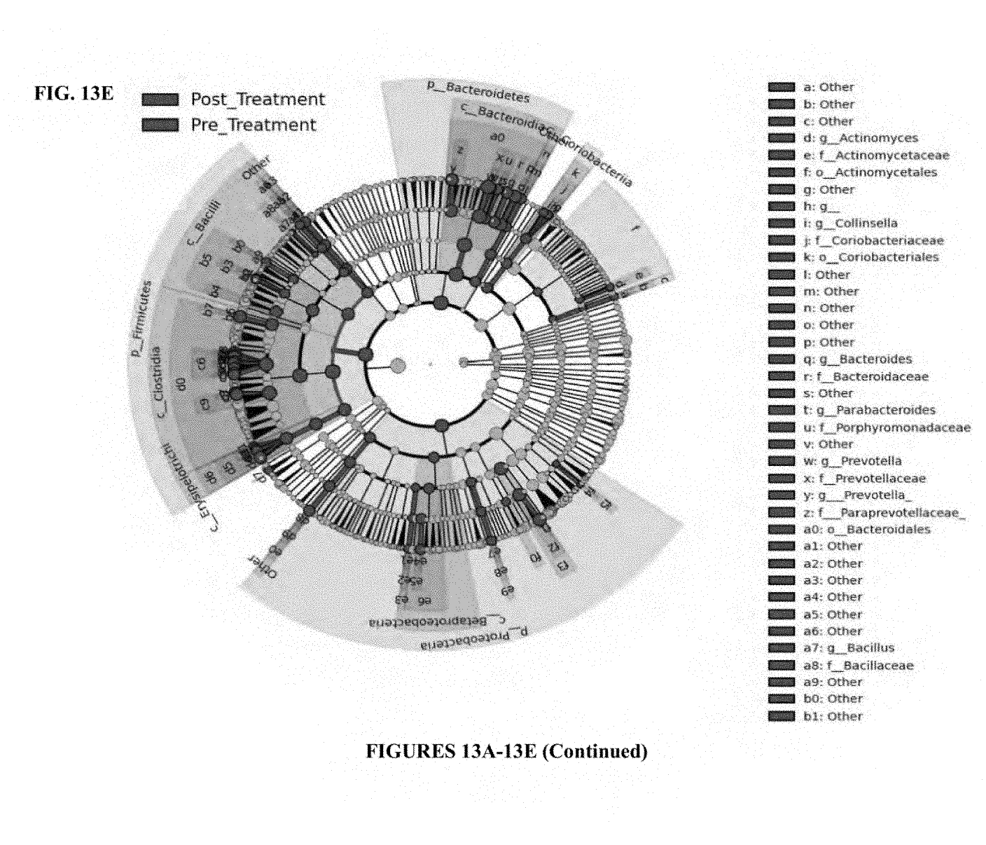

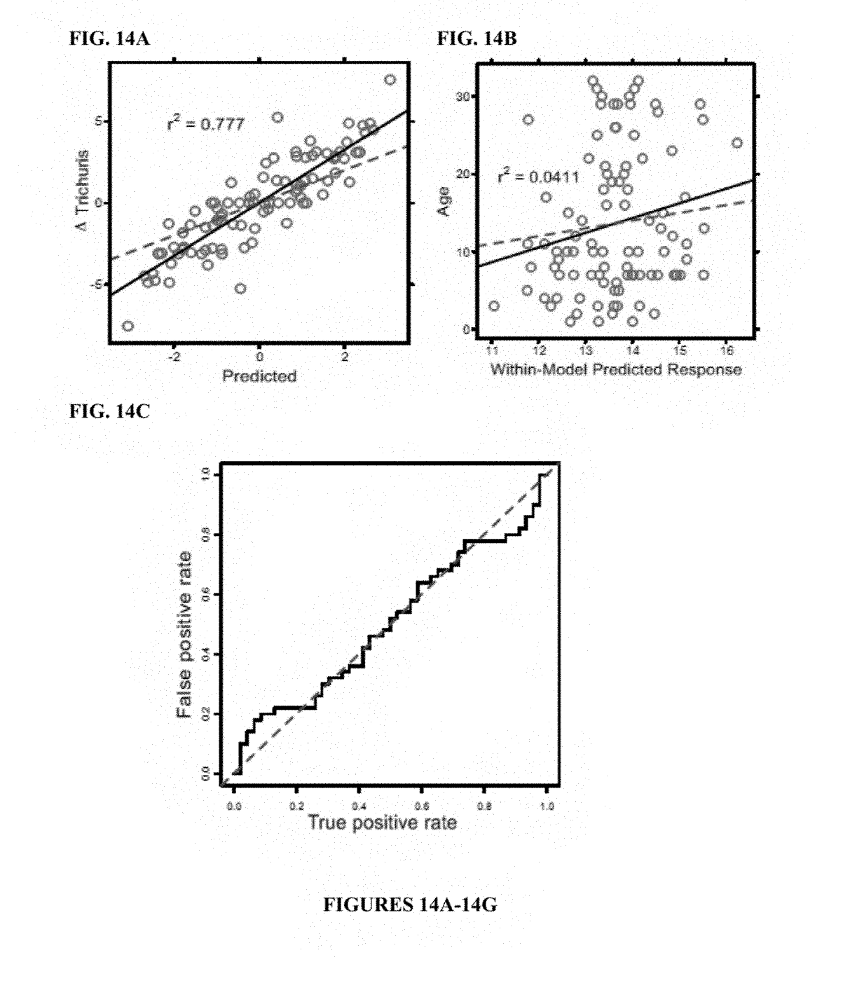

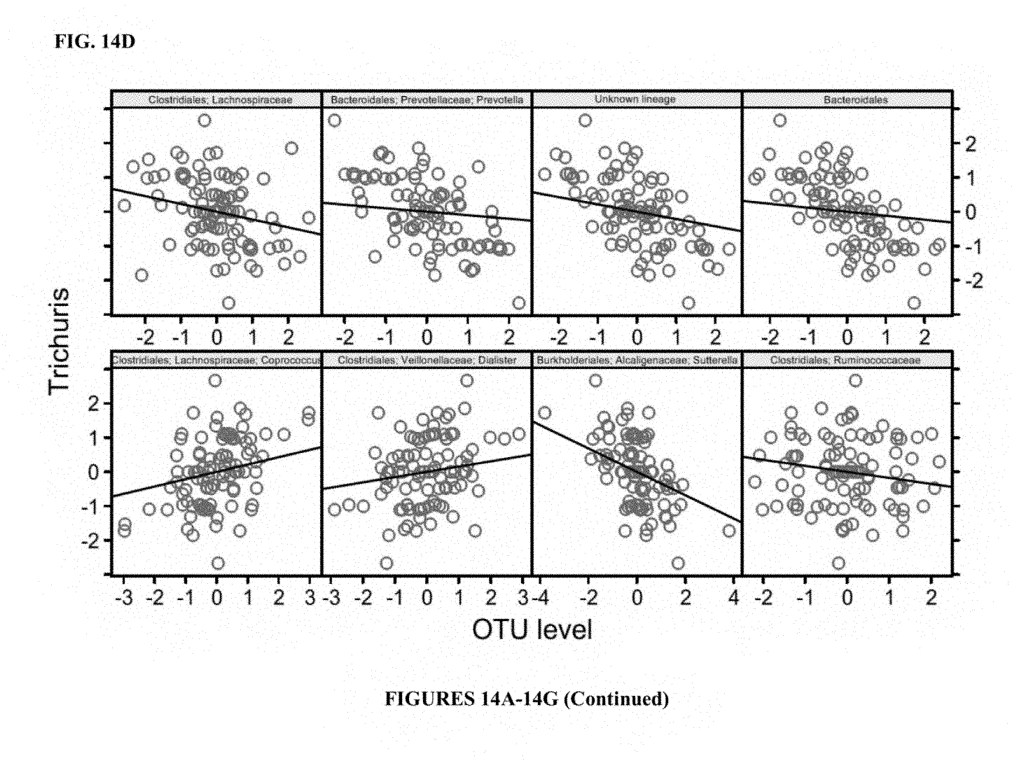

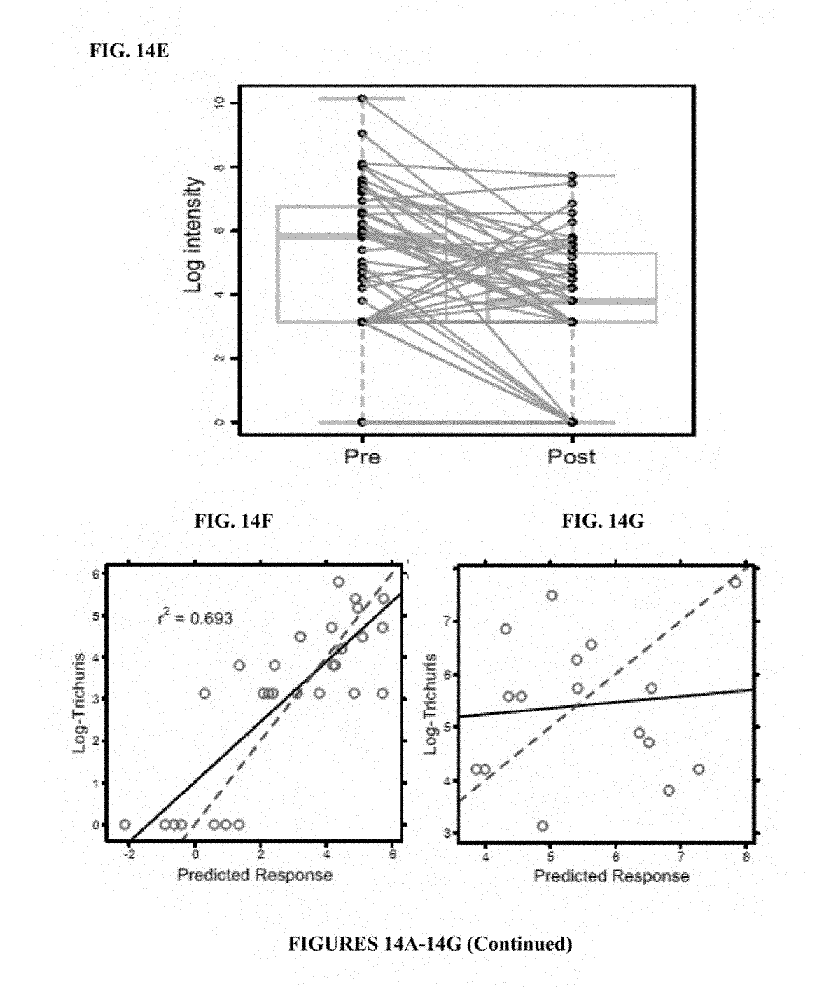

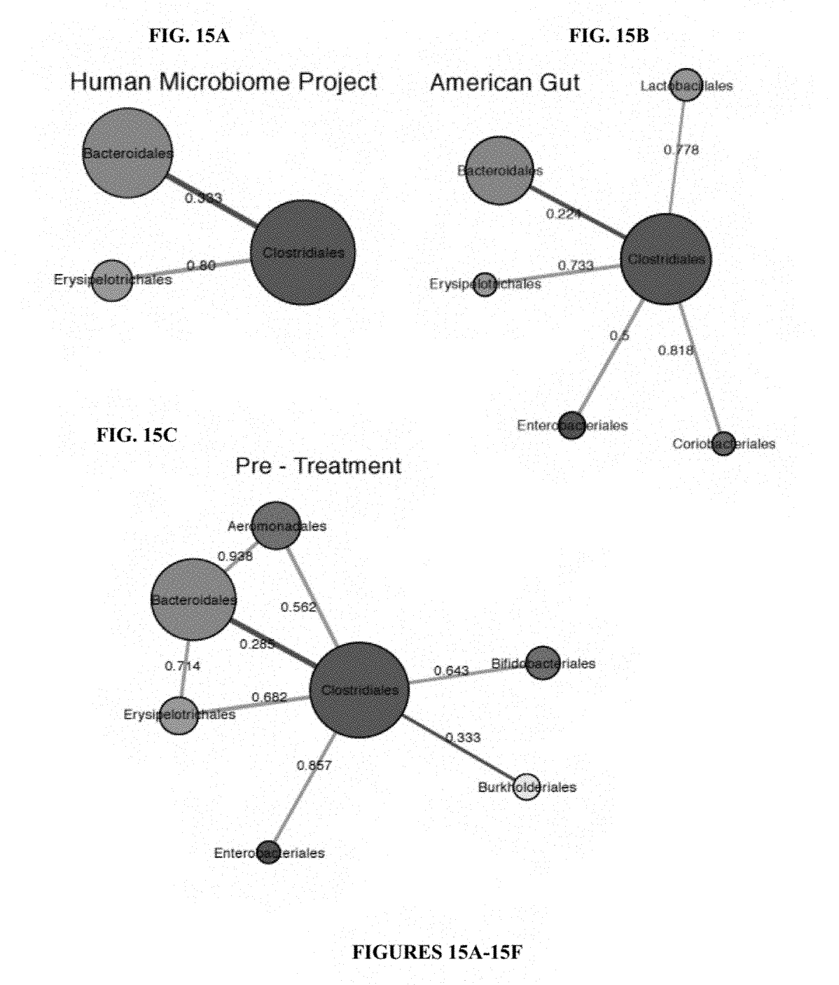

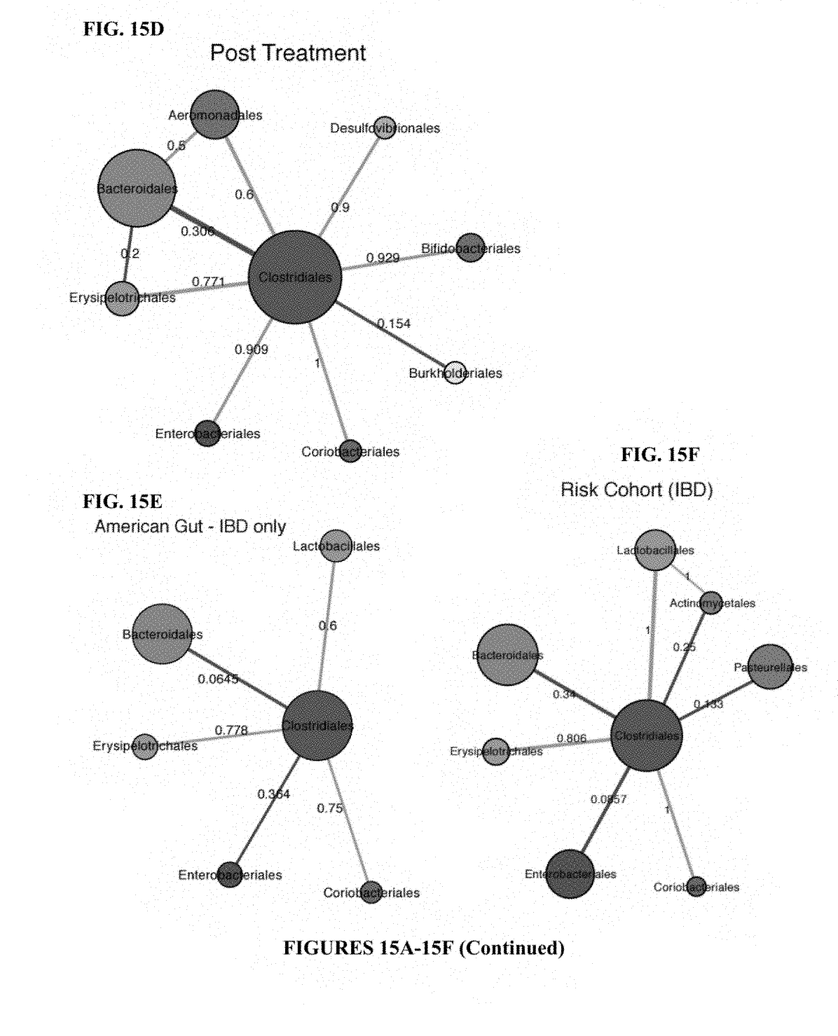

To control for factors other than helminth colonization (e.g., diet), stool samples collected from the Orang Asli were analyzed before and after deworming treatment with Albendazole (FIG. 13A, 13B, Table 3). Alpha-diversity of microbial communities was significantly reduced following treatment (FIGS. 4F, 13C, 13D). By LEfSe, Clostridiales was the most significantly reduced order, whereas Bacteroidales (Prevotella) was significantly expanded post treatment (FIGS. 4C-4E, 13E). Utilizing the egg burden data, Centered Log-Ratio (CLR) transformation was combined with Partial Least Square (PLS) regression to examine within subject changes, incorporating a repeated measures design (48). The resulting model showed that changes in Trichuris trichiura egg burden post treatment within individuals are strongly associated with a small set of bacterial taxa, independently of age and gender (FIGS. 4G, 14A-14C, Table 4). Specifically, Dialister and Coprococcus are two members of the order Clostridiales positively associated with changes in egg burden, whereas the Bacteroidales species Prevotella and another OTU are negatively associated (FIGS. 4H, 14D). Individuals without reduced egg burden did not show these changes in the microbiome, indicating that these findings are unlikely to be due to non-specific effects of Albendazole treatment (FIG. 14E-14G). Overall, these data support the hypothesis that helminth infection promotes the expansion of Clostridiales communities that outcompete Bacteroidales communities, although the T_{H}2 response was not examined here. Finally, a method (SPIEC-EASI) for inference of microbial ecological networks (19) was applied to publicly available human microbiome datasets consisting of healthy USA residents (Human Microbiome Project and American Gut Project) and pediatric IBD patients (RISK cohort) (20-22) and, based on the analysis, the antagonistic relationship between Clostridiales and Bacteroidales was found to be the most consistently observed negative relationship (FIGS. 4I-4J, 15).

Materials and Methods

Mice. Nod2−/−, Rag1−/−, Nod2−/−, and Stat6−/− mice on the C57BL/6 background were previously described and bred onsite in an MNV/Helicobacter-free specific pathogen free (SPF) facility at NYU School of Medicine (6, 23). Wild-type (WT) C57BL/6 mice were purchased from Jackson Laboratory and bred onsite to generate controls for experiments. Bone marrow chimeras were generated by lethally irradiating 8-week old female recipient mice (1100 CGy in two divided doses) followed by intravenous (i.v.) injection of 5×106 T cell-depleted bone marrow cells from donor female mice. For anti-CD4 treatment, mice were infected with T. muris and injected intra-peritoneally (IP) with 0.25 mg anti-CD4 (clone GK1.5) or anti-IgG2b isotype control (clone LTF-2) (BioXCell) every 3 days for 21 days and animals were sacrificed on day 21. For anti-IL13 treatment, mice were infected with H. polygyrus and injected IP with 0.15 mg anti-IL13 (clone 1316H) (eBioscience) or anti-IgG1 isotype control (clone 43414) (R&D Systems) starting on day 3 post-infection every 3 days for 12 days and animals were sacrificed on day 12. For recombinant IL-13 and IL-4 experiments, mice were injected IP with a complex of 25 μg of anti-IL13 (eBio1316H) and 5 μg of recombinant IL-13 (Peprotech cat #210-13), or a complex of 25 μg of anti-IL4 (BioXcel) and 5 μg of recombinant IL-4 (Peprotech cat #214-14) for 21 days and mice were sacrificed on day 21.

For co-housing experiments, 2 mice treated with T. muris were placed in the same cage with 3 uninfected mice for 35 days. All animal studies were performed according to protocols approved by the NYU School of Medicine Institutional Animal Care and Use Committee (IACUC).

Human Studies. The human study with ethical considerations was approved and granted (i.e., MEC Ref. No. 824.11 and No. 943.14) by the Ethics Committee of the University Malaya Medical Centre (UMMC), Malaysia, prior to the commencement of the study. The fieldwork was approved by the Department of Orang Asli Development (JAKOA) and prior permission was obtained from the Tok Batin (chieftain) of the Kuala Pangsun village before the study was conducted in the village. Kuala Pangsun village (101.88° E longitude, 3.21° N latitude) is situated in Hulu Langat district, the fifth largest district in Selangor state, Malaysia. The selection of this village was favorable in terms of logistics and feasibility, coupled with good cooperation from the villagers. In order to assess the effect of helminths on microbial communities, a pre-post study was conducted among the 53 subjects in the Kuala Pangsun village. The villagers were requested to be involved in the pre-treatment study in May 2013, which involved the examination of intestinal helminth infections in these villagers before anthelmintic treatment was administered to them. One week after the pre-treatment screening, a triple oral dose of albendazole (ABZ) tablets (3×400 mg, given over 3 consecutive days), produced by GlaxoSmithKline (London, UK), was distributed to all participants. The post-treatment fecal samples were collected 20 days after treatment administration. A 21-day time point was selected to examine the short-term effects of intestinal helminth expulsion after anthelmintic treatment on gut microbiota.

Prior to sample collection, their consent was obtained in written form either through signature or thumbprint. Then, the participants were given screw-capped containers labeled with names. Containers with participants' samples were assembled back the following day and immediately frozen on dry ice. Fecal samples were then transported using dry ice to the Department of Parasitology, Faculty of Medicine, University of Malaya. Then, fecal samples collected in screwcapped containers were separated into two portions: a) preserved in 2.5% potassium dichromate (Sigma) and stored at 4° C. (for intestinal helminth screening), b) stored in 1.5 ml microcentrifuge tube and kept in −80° C. prior to DNA extraction steps (for microbiome analysis). Formalin-ether sedimentation technique was used to determine the presence and absence of the intestinal helminth as previously described (24). The intensity of infection was determined by Kato Katz technique and results were recorded as egg per gram (epg), using the cut off determined by the WHO (3). Then, the fecal smears were observed using a light microscope under the magnification of 100× and 400× and the cure rate (percentage of helminth infected individuals who became egg-negative after albendazole treatment) was determined. In addition, 19 stool samples were collected randomly from University of Malaya as urban controls for this study with 6 Chinese, 1 Sabahan, 1 Sarawakian, 7 Indians, 2 Malays, and 2 Yemeni. Although of different races/nationality, these participants have been residing in Kuala Lumpur for over 5 years, and were exposed to a similar variety of food.

Microscopy and Flow Cytometry. Intestinal sections were prepared and stained with PAS/Alcian blue as previously described (1). The number of granule-containing goblet cells in the villi were quantified by counting positively-stained cells displaying the characteristic goblet morphology (1). Immunohistochemistry analysis of pSTAT6 was performed on formalin fixed paraffin-embedded small intestinal sections and the number of pSTAT6 cells in each frame under 20× magnification were quantified. Immuno-fluorescence analysis of Reg3β was performed by staining formalin fixed paraffin-embedded small intestinal sections, which were imaged using the Zeiss Axioplan epifluorescence microscope (Zeiss) and quantified using ImageJ as previously described (1). Intra-epithelial and lamina propria lymphocytes were isolated from the small intestine and colon as previously described (1). Lymphocytes were stimulated for 4 hours with a cell stimulation cocktail of PMA, ionomycin, brefeldin A and monensin from eBioscience. Stimulated cells were stained with anti-CD3ε PerCP, anti-TCRβ PE-Cy5, anti-CD8α PE-Cy7, anti CD4 APC-Cy7, anti-IFNγ APC, anti-IL17 PE, anti-IL4 APC and their respective isotype controls from Biolegend, and, anti-IL-13 FITC, anti-IL-10 PE and their respective isotype controls from eBioscience. Fixation and permeabilization buffers from Biolegend were used for intracellular cytokine staining, and a fixable live/dead stain from Biolegend was used to exclude dead cells. For nuclear staining, unstimulated cells were stained with anti-CD45 PerCP, anti-TCRβ BV510, anti CD4 APC-Cy7, anti-Ki67 Alexa700, anti-CD25 APC, anti-Neuropilinl BV421, anti-Helios PE-Cy7, anti-RORγt PE, anti-Foxp3 FITC, and their respective isotype controls from Biolegend using the Foxp3 staining kit (eBioscience). Flow cytometric analysis was performed on an LSR II (BD Biosciences) and analyzed using FlowJo software from TreeStar.

Bacteria. Fecal and tissue-associated B. vulgatus were quantified by dilution plating on selective BBE agar (BD) for 24-48 hours in an anaerobic chamber (AS-580, Anaerobe Systems). To quantify tissue-associated bacteria, one cm of small intestinal (ileum) tissue was flushed with PBS, cut open longitudinally, washed in PBS and homogenized. For inoculation into mice, B. vulgatus (1) and L. johnsonii (16) were anaerobically cultured at 37° C. in PYG broth for 48 hrs and Lactobacilli MRS broth for 24 hrs, respectively. The mix of 17 human Clostridia species previously described (15), were anaerobically cultured at 37 oC in PYG broth for 48-96 hours individually and combined prior to inoculation. Mice were orally gavaged with a 100 μl broth solution containing 1×10̂8 cfu Clostridia mixture, 1×10̂8 cfu L. johnsonii, or sterile broth every 3 days for 3 weeks. For in vitro mucus experiments, partially purified mucin from porcine stomach (M1778, Sigma Aldrich), predominantly consisting of muc2, was dissolved in a solution of 0.1M sodium acetate (pH 5) at a concentration of 20 mg/ml. Equal amounts of Clostridiales species (Clostridium (#28), Ruminococcus (#13), and Erysipelatoclostridium (#18)) or B. vulgatus were added to PYG broth with increasing concentrations of mucin (50, 100, 250 or 500 μg/ml) or vehicle control (0.1M sodium acetate) and plated at 1, 3, 6, and 9 hours post addition of mucin.

Helminth Infection. Maintenance of the T. muris lifecycle was carried out as described previously (25). Mice were infected with ˜25 embryonated eggs by oral gavage and sacrificed at D35 post infection. Worm burdens were assessed as described previously (26). In all conditions analyzed, successful chronic infection was established and equal worm burdens were confirmed. For Heligmosomoides polygyrus infection (27), mice were infected with approximately 200 L3 larvae via oral gavage and sacrificed at D12 (acute) or D35 (chronic) post infection.

Piroxicam Treatment. T. muris infected and uninfected WT and Nod2^{-/-} mice were treated with 60 mg/250 g and 80 mg/250 g of piroxicam as previously described (1). H&E stained small intestinal sections of mice treated with piroxicam were used for histopathological scoring in a blinded fashion. Each mouse was given an individual cumulative score based on the following criteria: number of focal ulcers (0=none, 1=1, 2=2 and so on), number of abscesses (0=none, 1=1, 2=2 and so on), the extent of epithelial hyperplasia (0=none, 1=elongated villi and crypts, 2=severe hyperplasia where the crypt villus axis is 2 times higher than the crypt villus axis in untreated mice), the presence of immune infiltrates (0=none, 1=pericryptal infiltrates, 2=submucosal infiltrates), and villus blunting (0=none, 1-2=moderate blunting, 3-4=severe blunting). The presence of macroscopic abnormalities such intestinal bleeding, and/or intestinal perforation in each mouse was also tabulated. For bacterial translocation assay, one cm of flushed small intestinal ileum, the entire spleen, and MLNs from piroxicam treated mice were homogenized and plated on blood agar plates and incubated for 24 hours at 37° C.

Gene Expression Analysis. RNA was isolated from one cm of small intestinal tissue (ileum) from Nod2^{-/-} mice treated with rIL-13 or PBS, or WT mice treated with PBS for normalization. An RNA library was prepared using the TruSeq stranded total RNA, with the RiboZero Gold kit and samples were sequenced on the Illumina HiSeq to generate single-end 50 bp reads. Raw sequencing reads were aligned to the mouse reference genome mm10 and the RefSeq reference transcriptome using TopHat (v2.0.12) with the alignment parameter—library-type fr-firststrand (28). Reads with mappability score (MAPQ) <30 were removed. The total number of filtered reads were counted for each gene using htseq-count with the parameters—stranded=reverse and—mode=union. Singleton genes (i.e. genes with total count <1) were filtered and the resulting count matrix was used for differential analysis using the DESeq2 workflow with default parameters (29, 30). Differential genes with rIL-13 treatment were identified at FDR 10%. Pathway analysis was performed using the DAVID platform as previously described (6). This data has been made publicly available and can be accessed using the GEO accession number GSE76504.

16S Library Preparation. DNA was isolated from stool samples using the NucleospinSoil Kit (Macherey-Nagel). Bacterial 16S rRNA gene was amplified at the V4 region using a modified protocol from Caporaso et al (31). The forward primer construct contained the 5′ Illumina adapter, the forward primer pad, a two-base linker (‘GT’) and the 515F primer (5′-AAT GAT ACG GCG ACC ACC GAG ATC TAC ACT ATG GTA ATT GTG TGC CAG CMG CCG CGG TAA −3′) (SEQ ID NO:4). The reverse primer construct contained the 3′ Illumina adapter, a unique 12-base error correcting Golay barcode, the reverse primer pad, a two-base linker sequence (‘CC’) and the 806 R primer (5′-CAA GCA GAA GAC GGC ATA CGA GAT NNN NNN NNN NNN AGT CAG TCA GCC GGA CTA CHV GGG TWT CTA AT -3′) (SEQ ID NO:5). Cycling protocol consisted of 94° C. for 3 min, 35 cycles of 94° C. for 45s, 50° C. for 60s and 72° C. for 90s, with a final extension of 72° C. for 10min. Amplification was performed in triplicates and the pooled amplicon was purified with QIAgen PCR purification kit. The purified amplicon library was pooled at equimolar ratio and sequenced on the Illumina MiSeq with a 2×150 cycle run (Illumina, San Diego Calif., USA).

16S Sequences Analyses. Sequencing read mates of the 16S library were joined using the fastqjoin function from EA-utils (32). The joined reads were processed using the Quantitative Insights Into Microbial Ecology (QIIME) software package (33). The split library.py function was first used for demultiplexing, in addition to performing quality filter with default parameters (minimum quality score of 25, minimum/maximum length of 200/1000, no ambiguous bases allowed and no mismatches allowed in the primer sequence). Operational Taxonomic Units (OTUs) were defined using a combination of closed reference and de novo sequence clustering methods (pick_open_reference_otus.py workflow in QIIME). The Greengenes reference collection (version 13_5) was used as reference sequences and similarity threshold was defined at 97%. The resulting OTU table was filtered for singletons before downstream analyses. Alpha diversity analysis was done using the metrics observed OTUs, Shannon index and chaol (34, 35).

The QIIME alpha rarefaction workflow (alpha_rarefaction.py) was used with default parameters on an OTU table that was first rarefied to the minimum sampling depth. Beta diversity was calculated using unweighted UniFrac distance performed on an uneven OTU table (36, 37). Principle Coordinate Analyses (PCoA) was performed on the UniFrac distance matrix and the resulting PCoA plot visualized using the Emperor graphics program (38). The LDA Effect Size (LEfSe) algorithm (http://huttenhower.sph.harvard.edu/galaxy/) was used to identify differentially abundant taxa in different biological groups at a threshold LDA score described in the legends (39).

Statistical Analysis. An unpaired two-tailed t test was used to evaluate differences between two groups. A paired two-tailed t test was used to evaluate differences between different time points in the same group. An ANOVA was used to evaluate experiments involving multiple groups with the Holm-Sidak multiple comparisons test. For contingency tables, Fisher's exact test was used. For experiments requiring non-parametric analyses, the Wilcoxon-Mann-Whitney test was used. For weight loss analysis, an ANOVA with the Holm-Sidak multiple comparisons test was used to evaluate the area under the curve for each individual mouse.

Bioinformatic Analysis

### Sparse and Compositionally-Robust PLS Regression

The inventors sought to detect associations between specific taxa in fecal microbiota communities and hostside measures, while reducing the detection of statistically spurious associations. This pipeline was (i), the compositionally-robust centered log-ratio transformation (clr) (40) of OTU relative abundance data (with a single pseudocount added prior to normalization) and (ii), estimation of a sparse linear model via Partial Least Squares (PLS) regression, to model high-dimensional and multi-collinear feature/responses (e.g. OTUs, taxa, and host covariates). Categorical variables were dummy coded into values of (−1 or 1) to indicate if a sample belongs to that class. L1-penalized partial least squares regression (41-43) was then applied to fit a bi-linear model. The number of latent components in the sPLS model are fixed to the number of non-zero singular values in the cross-covariance matrix. Model sparsity is controlled via the scalar parameter η that weights the influence of the L 1 penalty.

A two-stage approach was used to find a sparse set of significant OTU-phenotype associations. In the first stage, a stability selection approach was used for regularization selection (StARS (44)) to determine the sparsity parameter η; StARS has been previously shown to be competitive for graphical model problems of similar complexity and scale (44). The sPLS model was rebuilt over 100 random subsets of the data over a range of values for calculating the fraction of data subsets that included a given OTU in the support (i.e., the nonzero model coefficients) at each η. A summary statistic of overall model variability was computed to select the most stable model that exceeds the variability threshold (0.1%) (44). In the second stage, the statistical significance of individual OTUs in the model was assessed by computing empirical p-values over 30,000 bootstrapped PLS models on the StARS-selected support. The models were then compared to an empirical null model (generated by fitting randomized permutations of the data), which yields a p-value for each OTU-host phenotype pair.

For the experiments that relied on repeated measures design (i.e. Trichuris deworming), an additional step was incorporated into the pipeline. The dr-transformed OUT compositions as well as host responses data were decomposed into the relevant ‘within-subject’ components using a one-factor variance decomposition (45). The within-subject component captures experimental perturbation effects by subtracting between-subject variances, and is directly proportional to the change in data levels over the single repeated measure. The sPLS model was applied directly to the within-subject variances as described (45) and implemented in the mixOmics package in R (46). Routines from the spls and caret libraries in R were used and a custom package was developed (which includes methods for the full pipeline, a similar approach for discriminant analysis (17) and biplots) called compPLS [software and additional methods are available at http://github.com/zdk123/compPLS].

This approach identifies a small set of bacterial OTUs for which a linear combination of abundance changes accurately models concurrent changes in Trichuris trichuria egg burden (ΔTrichuris). Though this is a multivariate model, individual OTU are reported×Δ Trichuris relationships by filtering empirical p-values of the model coefficient (a=10-2) and the direction of association from sign of the coefficient (Table 4).

These results were visualized in several ways. For continuous responses, such as Δ Trichuris, the within-sample prediction error (r2) was shown in a residual plot. For categorical responses (e.g. Sex), the model predicts a continuous value, fit to the dummy coded value. To show within-model error for classification, a varying threshold of these values was used as a cutoff to predict membership in this class and the True Positive vs False Positive rate (Receiver Operator Characteristic—ROC—curve) is reported as this threshold is varied. Useful models show better than random classification (AUC=0.05).

Finally, to visualize pairwise relationships between taxa and ΔTrichuris, data fit to the model is shown (i.e. decomposed within-subject variances, scaled and centered) as scatterplots. Model fits are shown as overlaid straight lines, slopes are fitted model coefficients, to show the relative contribution of the OTU to the overall prediction. Multivariate relationships were visualized between OTUs and ATrichuris learned from the sPLS model using biplots. The subspace where OTUs and response maximally covaries is learned by the PLS model, the data is projected onto the loadings (sparse set of OTUs), which are the model ‘scores’. Loadings are visualized as vectors, representing each OTU, and colored at the order level, length of each vector is proportional to the contributing variance of that OUT and the angle between each loading vector indicates the correlation between those OTUs in the respective space (0°—perfect correlation, 90°—no correlation, 180° perfect anti-correlation). Each PLS component represents the linear combination of OTUs contributing to the OTU—ΔTrichuris covariation in that space, and since each successive PLS component is learned from the residuals of the data projected onto the previously-learned component, PLS components are orthogonal.

### Estimation of Microbial Association Networks

Several publicly available datasets were selected to compare to the Orang Asli (OA) (n=45 before and after deworming) microbial networks. These were American Gut Project (AGP) (22) (fecal, n=3671), AGP samples [self-identified] IBD only (fecal, n=178), the Human Microbiome Project (HMP) data (v35 reads) (21) (fecal, n=402) and the RISK IBD cohort (20) (fecal and GI biopsies, n=913). AGP and HMP project data was obtained from biocore project [https://github.com/biocore/American-Gut] and RISK cohort microbiome data was obtained from the Qiita repository [http://qiita.microbio.me/] (Study IDs 1939 and 1998). OTUs that appeared in fewer than 37% (chosen for consistency with previous studies of AGP (19)) of samples across the entire dataset were filtered out for this analysis. Sparse Inverse Covariance estimation for Ecological Association Inference (SPIEC-EASI) (19) was applied to examine the network model from each cluster, where nodes in the network are OTUs and edges are inferred relationships between OTUs it its environment. SPIEC-EASI was run using Meinshausen-Bühlmann (MB) neighborhood selection, and network model selection via StARS, using a variability threshold of 0.05%. Over the set of OTUs retained (p) the number of edges inferred (e) for each dataset was: OApre, p=422 and e=2143, OA-post, p=424 and e=2048; AGP, p=243 and e=781; AGP-IBD, p=152 and e=327; HMP, p=108 and e=141; RISK, p=246 and e=890.

To characterize relationships at the order level, both positive and negative model coefficients were summarized (symmetrizing MB edges by taking the maximum absolute value of coefficient) between OTU pairs. The fraction of positive edges between all OTUs of the same order was reported in a network diagram, plotting edge color and the majority sign (green—positive, red—negative). Node size is proportional to total number of edges for that order (therefore reflecting the number of taxa as well as overall connectivity).

Classification of Orang Asli into Responders and Non-Responders to Treatment

The response to treatment among Orang Asli samples was classified by measuring the difference between log-transformed Trichuris levels (with a single pseudocount added to counts) before and after treatment. For patient samples that had parasites prior to deworming, change in Trichuris burden was partitioned around randomly selected cluster mediods (pam) and found that response to deworming clustered into two distinct groups based on a threshold of −Δ0.03 (above which was classified as non-responders and the rest being classified as non-responders). Using after treatment samples, an sPLS model was fit to dr-transformed compositions to predict log-transformed Trichuris trichuria worm burden. The within-sample fit among responders (r2=0.693) was compared to the out-of-sample fit of Non-responders (r2=0.01).

Creation of Cytokine-Fc Fusion Proteins

Fc-fusion proteins were generated to increase the half-life of Type 2 cytokines for usage in vivo to promote the growth of Clostridiales and to inhibit Bacteroides. The fusion proteins were produced in serum-free CHO-K1 cell lines, which result in a different glycosylation pattern, which helps to maintain prolonged serum half-life. All the fusion proteins have identical mouse IgG1 Fc regions. The protein sequences of the fusion proteins are listed below.

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTFRSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH

Use of Cytokine-Fc Fusion Proteins

WT and Nod2^{-/-} mice will be treated with IL4-fc, IL13-fc and IL33-fc fusion proteins to determine if colonization with Bacteroides vulgatus is inhibited by treatment with the fusion proteins. Stool samples will also be monitored for expansion of Clostridial strains previously identified by 16S sequencing. It will also be determined if treatment with the fusion proteins increased M2 macrophages in the gut. M2 macrophages in the gut will be determined by Flow Cytometry of cell surface markers (PD-L2, CD301, CD206, F4/80, for example and not limitation) and by RT-PCR of marker genes (including for example and not limitation, Arginasel, Fizzl/Relma, Ym1/Chi3l3). IL4-fc, IL13-fc and IL33-fc fusion proteins will be also delivered to animals in combination with 17 human Clostridial strains to determine if there are synergistic effects on inhibiting Bacteroides vulgatus and reducing intestinal inflammation.

TABLE 1
Nod2^{-/-} mice treated with rIL-13 display an increase in M2 macrophage
activation genes. List of genes upregulated in Nod2^{-/-} mice treated with
recombinant IL-13 compared to PBS controls.

| Ref Seq | | | | |
|---|---|---|---|---|
| (GenBank | Log₂ Fold | | Gene | |
| Accession No.) | Change | p-value | Symbol | Description |
| NM_001081957 | 1.530004201 | 3.48E−05 | Gm11428 | activated |
| | | | | macrophage/ |
| | | | | microglia |
| | | | | WAP domain |
| NM_007482 | 1.46752967 | 1.92E−05 | Arg1 | arginase-1 |
| NM_009252 | 1.580151604 | 9.40E−06 | Serpina3n | serine protease |
| | | | | inhibitor A3N |
| | | | | precursor |
| NM_009690 | 1.504428477 | 8.47E−05 | Cd5l | CD5 antigen-like |
| | | | | precursor |
| NM_009892 | 2.680582506 | 3.05E−12 | Chi3l3 | chitinase-3-like |
| | | | | protein 3 precursor |
| NM_011315 | 1.846496689 | 6.94E−07 | Saa3 | serum amyloid A-3 |
| | | | | protein precursor |
| NM_020509 | 1.625305541 | 2.67E−05 | Retnla | resistin-like alpha |
| NM_021443 | 1.499722438 | 8.21E−05 | Ccl8 | C-C motif |
| | | | | chemokine 8 |
| | | | | precursor |
| NM_053113 | 1.697677309 | 5.51E−06 | Ear11 | non-secretory |
| | | | | ribonuclease |

Table 2 represents the incidence of different intestinal helminths among the rural Orang Asli individuals before deworming treatment, and urban individuals residing in Kuala Lumpur.

TABLE 2
Prevalence of intestinal helminth infections among Orang Asli and urban individuals.

| | Pre-Treatment (N = 75) | | | Urban individuals (N = 19) | | |
|---|---|---|---|---|---|---|
| | Positive | Prevalence | | Positive | Prevalence | |
| | samples | (%) | 95% CI | samples | (%) | 95% CI |
| Intestinal | 72 | 96.0 | 91.57-100.43 | 1 | 5.3 | −4.80-15.40 |
| helminths | | | | | | |
| T. trichiura | 70 | 93.3 | 87.68-98.98 | — | — | — |
| Hookworm | 27 | 36.0 | 16.95-37.05 | 1 | 5.3 | −4.8-15.40 |
| Ascaris | 10 | 13.3 | 5.64-21.02 | — | — | — |
| spp. | | | | | | |

Table 3 representing de-worming efficacy in the Orang Asli population pre-treatment (n=75) and posttreatment (n=64).

TABLE 3
Efficacy of albendazole in treating intestinal helminth infections
according to type of parasites among Orang Ash i in Kuala Pangsun.

| Infection intensity | | | | | | |
|---|---|---|---|---|---|---|
| (%) | T. trichiura | | Ascaris spp. | | Hookworm | |
| | Before Treatment (range of epg) | | | | | |
| | 22-25,718 epg^{b} | | 22-21,824 epg | | 22-1,999 epg | |
| Light-intensity | 50 | 66.7 | 7 | 9.3 | 27 | 36.0 |
| infections | | | | | | |
| Moderate-intensity | 19 | 25.3 | 3 | 4.0 | — | — |
| infections | | | | | | |
| Heavy-intensity | 1 | 1.3 | — | — | — | — |
| infections | | | | | | |
| Total | 70 | 93.3 | 10 | 13.3 | 27 | 36.0 |
| | After Treatment | | | | | |
| | 22-2,266 epg | | 22-4999 epg | | 22 | |
| Light-intensity | 45 | 70.3 | 2 | 3.1 | 1 | 1.6 |
| infections | | | | | | |
| Moderate-intensity | 2 | 3.1 | — | — | — | — |
| infections | | | | | | |
| Total | 47 | 73.4 | 2 | 3.1 | 1 | 1.6 |
| Cure rate (%)^{a} | 2 3 | 32.9 | 8 | 80.0 | 26 | 96 .3 |
| 20 days | | | | | | |

^{a}Number of infected persons cured after deworming/number of infected persons before deworming;
^{b}Eggs per gram of faecal sample.

Taking advantage of the repeated measures design, a sPLS model was used on within-subject variances of clr-transformed compositions and (log-transformed) Trichuris burden and it was found that the change in Trichuris burden is strongly associated with a minimal set of taxa. The resulting models show a clear positive association for intensity of T. trichiura infection with a negative association with Bacteroidia populations, Prevotella and Bacteroidales. Two Clostridia OTUs show a clear positive association, Coprococcus and Dialister, with Ruminococcaceae and Lachnospira showing the opposite association that is nevertheless orthogonal to Bacteroidia. While linear combinations of these selected taxa show robust within-model error (ΔTrichuris), no selected taxa was found to predict Age or Sex despite being included in the model. Patients that had had under deworming were classified as either responding or not responding to Trichuris deworming (using a cut off of −Δ0.03 log-Trichuris) and requiring the presence of Trichuris before deworming. It was found that regressing sPLS on log Trichuris burden was accurate at explaining worm burden (r2=0.693) but was not predictive of Trichuris levels in non-responders.

TABLE 4
Taxa selected by multi-level sPLS in response to deworming.

| | | | Trichuris |
|---|---|---|---|
| Tax ID | Phylum | Taxa | (p-value, sign) |
| OTU684 | | Bacteria | 1.333e−04 (−) |
| OTU927 | Bacteroidetes | Prevotella | 7.467e−03 (−) |
| OTU1185 | Bacteroidetes | Bacteroidales | 8.400e−03 (−) |
| 111135 | Proteobacteria | Sutterella | <3.333e−05 (−) |
| 317814 | Firmicutes | Ruminococcaceae | 8.000e−04 (−) |
| 4414476 | Firmicutes | Lachnospira | 1.067e−03 (−) |
| 182289 | Firmicutes | Coprococcus | 4.000e−04 (+) |
| 174516 | Firmicutes | Dialister | 6.734e−03 (+) |

Table 5 provides primers for the PCR detection of the 17 human Clostridia strains that can be used to monitor the effect of the type 2 cytokine/mucin treatment of the invention. Clostridia strains can be also monitored by 16S sequencing.

TABLE 5
Primers for Clostridia that can be used to monitor the effect of the type 2
cytokine/mucin treatment of the invention.

| Primer | | | | |
|---|---|---|---|---|
| No. | Orientation | Position | Primer Sequence (5′ to 3′) | Strain |
| 1 | Fwd | 31 | AGACCGCATAGGTAAAGA | Sacchorogumia |
| | | | TACC (SEQ ID NO: 6) | |
| | Rv | 110 | ATGCGCCATAAGTCCATCC | |
| | | | (SEQ ID NO: 7) | |
| 3 | Fwd | 25 | AACGGGGTGCTCATGAC | Clostridium |
| | | | (SEQ ID NO: 8) | viride/ |
| | | | | butyricicoccus / |
| | | | | anaerobacterium |
| | Rv | 149 | CGGTATTAGCACTCCTTTC | |
| | | | GG (SEQ ID NO: 9) | |
| 4 | Fwd | 24 | CGAGCGAAGCGGTTTCA | Clostridium |
| | | | (SEQ ID NO: 10) | |
| | Rv | 134 | TTCTAACTGTTATCCCCCA | |
| | | | GTGTA (SEQ ID NO: 11) | |
| 6 | Fwd | 23 | GAGCGAAGCAGTAAGACG | Blautia |
| | | | (SEQ ID NO: 12) | luti/coccoides/ |
| | | | | producta |
| | Rv | 130 | CTAACTGTTATCCCCCTGT | |
| | | | ATGA (SEQ ID NO: 13) | |
| 7 | Fwd | 5 | CGGCGTGCCTAACACAT | Clostridium/Blautia |
| | | | (SEQ ID NO: 14) | luti/ |
| | | | | ruminococcus |
| | Rv | 86 | GTCCGCCACTCAGTCAATC | |
| | | | A (SEQ ID NO: 15) | |
| 8 | Fwd | 24 | GTCGAACGAAGTGAAGAT | Clostridium |
| | | | AGC (SEQ ID NO: 16) | |
| | Rv | 112 | TTATCCCGGTCATACAGGC | |
| | | | (SEQ ID NO: 17) | |
| 9 | Fwd | 12 | TGGGGAACCTGCCCTATAC | Anaerostipes |
| | | | A (SEQ ID NO: 18) | hadrus |
| | Rv | 107 | CGGAGCTTTTCACACCGAA | |
| | | | T(SEQ ID NO: 19) | |
| 13 | Fwd | 25 | ACGGAGCTTACGTTTTGAA | Ruminococcus |
| | | | (SEQ ID NO: 20) | Albus |
| | Rv | 127 | GGCTGTTATCCCCCTCTGA | |
| | | | (SEQ ID NO: 21) | |
| 14 | Fwd | 33 | GCGCTGTTTTCAGAATCTT | Clostridium |
| | | | (SEQ ID NO: 22) | |
| | Rv | 195 | ACCGGAGTTTTTCACACTA | |
| | | | C (SEQ ID NO: 23) | |
| 15 | Fwd | 5 | CGGCGTGCCTAACACAT | Clostridium |
| | | | (SEQ ID NO: 24) | |
| | Rv | 83 | CGCCACTCAGTCATCTCAG | |
| | | | AA (SEQ ID NO: 25) | |
| 16 | Fwd | 22 | AGTCGAACGAAGCGATTT | Clostridium |
| | | | AAC (SEQ ID NO: 26) | symbiosum |
| | Rv | 194 | CCGGAGTTTTTCACACTGT | |
| | | | AT (SEQ ID NO: 27) | |
| 18 | Fwd | 22 | TCGAACGCGAGCACTTG | Erysipelato- |
| | | | (SEQ ID NO: 28) | clostridium |
| | Rv | 144 | CCATGCAGTGTCCGTACC | |
| | | | (SEQ ID NO: 29) | |
| 21 | Fwd | 45 | GCGCTTTACTTAGATTTCT | Clostridium |
| | | | TCG (SEQ ID NO: 30) | oroticum |
| | Rv | 187 | CCATGCGGTACTGTGGT | |
| | | | (SEQ ID NO: 31) | |
| 26 | Fwd | 52 | GGAGATGAAGGCGGCT | Clostridium |
| | | | (SEQ ID NO: 32) | scindens/Rumino |
| | | | | coccus faecis |
| | Rv | 281 | ACCCTCTCAGGTCGGC | |
| | | | (SEQ ID NO: 33) | |
| 27 | Fwd | 20 | GCAGTCGAACGGAGTTAT | Clostridium |
| | | | G (SEQ ID NO: 34) | saccharolyticum / |
| | | | | Blautia producta |
| | Rv | 182 | CACACTGCCTCATGTGAAG | |
| | | | (SEQ ID NO: 35) | |
| 28 | Fwd | 20 | CAGTCGAACGAAGCATCT | Clostridium |
| | | | TATAG (SEQ ID NO: 36) | aldenense / |
| | | | | Blautia / |
| | | | | Ruminococcus |
| | Rv | 209 | GATCCATCTCACACCACCT | |
| | | | (SEQ ID NO: 37) | |
| 29 | Fwd | 777 | GGTGTAGGTGGGTATGGA | Clostridium |
| | | | C (SEQ ID NO: 38) | aldenense / |
| | | | | Blautia / |
| | | | | Ruminococcus |
| | Rv | 974 | AAATCCTCTTTACAGGAGC | |
| | | | G (SEQ ID NO: 39) | |
| Eubacteria | UniF340 | | ACTCCTACGGGAGGCAGC | Infect. Immun. |
| (all | | | AGT (SEQ ID NO: 40) | March 2008 vol. |
| bacteria) | | | | 76 no. 3 907-915 |
| | UniR514 | | ATTACCGCGGCTGCTGGC | |
| | | | (SEQ ID NO: 41) | |

### Example 2

The effect of mucin consumption on Clostridia expansion in Nod2^{-/-} mice was examined. In this study, germ-free Nod2^{-/-} mice on the C57BL/6 background were previously described and bred onsite in an MNV/Helicobacter-free specific pathogen free (SPF) facility at NYU School of Medicine (6, 23). The mice were provided free access (24 hours a day) to either control/regular drinking water or drinking water contining mucin (75 μg/ml in water) obtained from porcine stomach and consisting of primarily MUC2 (Sigma M1778). Seven days later the germ-free mice were gavaged with a Kenya Honda mixuture, which consists of a rationally selected mixture of Clostridia strains from the human microbiota. In particular, 17-mix from K. Atarashi et al., Treg induction by a rationally selected mixture of Clostridia strains from the human microbiota. Nature 500, 232-236 (2013) was used. Atarashi et al., is incorporated herein by reference in its entirety.

On day 24, stool samples were collected from each group (4 control mice and 3 mucin treated mice), diluted with 1× phosphate-buffered saline (PBS) (10⁻¹-10⁶) and plated on brucella blood agar plates and incubated at 37° C. in a Coy anaerobic chamber, for 48 hours. All strains of Clostridia are able to grow on these agar plates. Mucin consumption lead to Clostridia expansion in the gut of the treated mice.

### Example 3

B. vulgatus is cultured anaerobically in peptone yeast glucose broth (Anaerobe Systems. Cat #AS-822) for 48 hours and Nod2^{-/-} mice are orally gavaged with 1×10⁷ CFU's of bacteria in 100 μl broth. Seven days following inoculation, when stable colonization is established in the gut, mice are injected i.p. with either 5 μg of recombinant IL-13-Fc fusion protein (SEQ ID NO:2) in 100 μl PBS, or just 100 μl PBS for controls. Injections are repeated every three days for 21 days. Stool is collected from all mice on each day of injection and dilutions (with 1× phosphate-buffered saline (PBS) (10⁻¹-10⁶)) are plated on selective Bacteroides Bile Esculin agar (BD. Cat #221836) and incubated at 37° C. in a Coy anaerobic chamber, for 48 hours to quantify B. vulgatus colonization.

List of Sequences:

| SEQ | | | |
|---|---|---|---|
| ID NO | Source | Type | Sequence |
| 1 | Synthetic | Protein | HIHGCDKNHLREIIGILNEVTGEGTPCTEMDVP |
| | | | NVLTATKNTTESELVCRASKVLRIFYLKHGKTP |
| | | | CLKKNSSVLMELQRLFRAFRCLDSSISCTMNES |
| | | | KSTSLKDFLESLKSIMQMDYSGGGGSVPRDCG |
| | | | CKPCICTVPEVSSVFIFPPKPKDVLMISLTPKVT |
| | | | CVVVDISKDDPEVQFSWFVDDVEVHTAQTKPR |
| | | | EEQINSTFRSVSELPILHQDWLNGKEFKCRVNS |
| | | | AAFPAPIEKTISKTKGRPKAPQVYTIPPPKEQMA |
| | | | KDKVSLTCMITNFFPEDITVEWQWNGQPAENY |
| | | | KNTQPIMDTDGSYFVYSKLNVQKSNWEAGNT |
| | | | FTCSVLHEGLHNHHTEKSLSHSPGAHHHHHH |
| 2 | Synthetic | Protein | PVPRSVSLPLTLKELIEELSNITQDQTPLCNGSM |
| | | | VWSVDLAAGGFCVALDSLTNISNCNAIYRTQRI |
| | | | LHGLCNRKAPTTVSSLPDTKIEVAHFITKLLSYT |
| | | | KQLFRHGPFGGGGSVPRDCGCKPCICTVPEVSS |
| | | | VFIFPPKPKDVLMISLTPKVTCVVVDISKDDPEV |
| | | | QFSWFVDDVEVHTAQTKPREEQINSTFRSVSEL |
| | | | PILHQDWLNGKEFKCRVNSAAFPAPIEKTISKT |
| | | | KGRPKAPQVYTIPPPKEQMAKDKVSLTCMITN |
| | | | FFPEDITVEWQWNGQPAENYKNTQPIMDTDGS |
| | | | YFVYSKLNVQKSNWEAGNTFTCSVLHEGLHN |
| | | | HHTEKSLSHSPGAHHHHHH |
| 3 | Synthetic | Protein | SIQGTSLLTQSPASLSTYNDQSVSFVLENGCYVI |
| | | | NVDDSGKDQEQDQVLLRYYESPCPASQ GDG |
| | | | VDGKKLMVNMSPIKDTDIWLHANDKDYSVEL |
| | | | QRGDVSPPEQAFFVLHKKSSDFVSFECKNLPGT |
| | | | YIGVKDNQLALVEEKDESCNNIMFKLSKIGGG |
| | | | GSVPRDCGCKPCICTVPEVSSVFIFPPKPKDVL |
| | | | MISLTPKVTCVVVDISKDDPEVQFSWFVDDVE |
| | | | VHTAQTKPREEQINSTFRSVSELPILHQDWLNG |
| | | | KEFKCRVNSAAFPAPIEKTISKTKGRPKAPQVY |
| | | | TIPPPKEQMAKDKVSLTCMITNFFPEDITVEWQ |
| | | | WNGQPAENYKNTQPIMDTDGSYFVYSKLNVQ |
| | | | KSNWEAGNTFTCSVLHEGLHNHHTEKSLSHSP |
| | | | GAHRHHHH |
| 4 | Synthetic | DNA | 5′ AAT GAT ACG GCG ACC ACC GAG ATC |
| | | | TAC ACT ATG GTA ATT GTG TGC CAG CMG |
| | | | CCG CGG TAA 3′ |
| 5 | Synthetic | DNA | 5′ CAA GCA GAA GACGGC ATA CGA GAT |
| | | | NNN NNN NNN NNN AGT CAG TCA GCC GGA |
| | | | CTA CHV GGG TWT CTA AT 3′ |
| 6 | Sacchorogumia | DNA | 5′ AGACCGCATAGGTAAAGATACC 3′ |
| | (bacteria) | | |
| 7 | Sacchorogumia | DNA | 5′ ATGCGCCATAAGTCCATCC 3′ |
| | (bacteria) | | |
| 8 | Clostridium | DNA | 5′ AACGGGGTGCTCATGAC 3′ |
| | (bacteria) | | |
| 9 | Clostridium | DNA | 5′ CGGTATTAGCACTCCTTTCGG 3′ |
| | (bacteria) | | |
| 10 | Clostridium | DNA | 5′ CGAGCGAAGCGGTTTCA 3′ |
| | (bacteria) | | |
| 11 | Clostridium | DNA | 5′ TTCTAACTGTTATCCCCCAGTGTA 3′ |
| | (bacteria) | | |
| 12 | Blautia | DNA | 5′ GAGCGAAGCAGTAAGACG 3′ |
| | (bacteria) | | |
| 13 | Blautia | DNA | 5′ CTAACTGTTATCCCCCTGTATGA 3′ |
| | (bacteria) | | |
| 14 | Clostridium/ | DNA | 5′ CGGCGTGCCTAACACAT 3′ |
| | Blautia (bacteria) | | |
| 15 | Clostridium/ | DNA | 5′ GTCCGCCACTCAGTCAATCA 3′ |
| | Blautia (bacteria) | | |
| 16 | Clostridium | DNA | 5′ GTCGAACGAAGTGAAGATAGC 3′ |
| | (bacteria) | | |
| 17 | Clostridium | DNA | 5′ TTATCCCGGTCATACAGGC 3′ |
| | (bacteria) | | |
| 18 | Anaerostipes | DNA | 5′ TGGGGAACCTGCCCTATACA 3′ |
| | (bacteria) | | |
| 19 | Anaerostipes | DNA | 5′ CGGAGCTTTTCACACCGAAT 3′ |
| | (bacteria) | | |
| 20 | Ruminococcus | DNA | 5′ ACGGAGCTTACGTTTTGAA 3′ |
| | (bacteria) | | |
| 21 | Ruminococcus | DNA | 5′ GGCTGTTATCCCCCTCTGA 3′ |
| | (bacteria) | | |
| 22 | Clostridium | DNA | 5′ GCGCTGTTTTCAGAATCTT 3′ |
| | (bacteria) | | |
| 23 | Clostridium | DNA | 5′ ACCGGAGTTTTTCACACTAC 3′ |
| | (bacteria) | | |
| 24 | Clostridium | DNA | 5′ CGGCGTGCCTAACACAT 3′ |
| | (bacteria) | | |
| 25 | Clostridium | DNA | 5′ CGCCACTCAGTCATCTCAGAA 3′ |
| | (bacteria) | | |
| 26 | Clostridium | DNA | 5′ AGTCGAACGAAGCGATTTAAC 3′ |
| | (bacteria) | | |
| 27 | Clostridium | DNA | 5′ CCGGAGTTTTTCACACTGTAT 3′ |
| | (bacteria) | | |
| 28 | Erysipelato | DNA | 5′ TCGAACGCGAGCACTTG 3′ |
| | (bacteria) | | |
| 29 | Erysipelato | DNA | 5′ CCATGCAGTGTCCGTACC3′ |
| | (bacteria) | | |
| 30 | Clostridium | DNA | 5′ GCGCTTTACTTAGATTTCTTCG 3′ |
| | (bacteria) | | |
| 31 | Clostridium | DNA | 5′ CCATGCGGTACTGTGGT 3′ |
| | (bacteria) | | |
| 32 | Clostridium / | DNA | 5′ GGAGATGAAGGCGGCT 3′ |
| | Ruminococcus | | |
| | (bacteria) | | |
| 33 | Clostridium / | DNA | 5′ ACCCTCTCAGGTCGGC 3′ |
| | Ruminococcus | | |
| | (bacteria) | | |
| 34 | Clostridium / | DNA | 5′ GCAGTCGAACGGAGTTATG 3′ |
| | Blautia | | |
| | (bacteria) | | |
| 35 | Clostridium / | DNA | 5′ CACACTGCCTCATGTGAAG 3′ |
| | Blautia | | |
| | (bacteria) | | |
| 36 | Clostridium / | DNA | 5′ CAGTCGAACGAAGCATCTTATAG 3′ |
| | Blautia / | | |
| | Ruminococcus | | |
| | (bacteria) | | |
| 37 | Clostridium / | DNA | 5′ GATCCATCTCACACCACCT 3′ |
| | Blautia / | | |
| | Ruminococcus | | |
| | (bacteria) | | |
| 38 | Clostridium / | DNA | 5′ GGTGTAGGTGGGTATGGAC 3′ |
| | Blautia / | | |
| | Ruminococcus | | |
| | (bacteria) | | |
| 39 | Clostridium / | DNA | 5′ AAATCCTCTTTACAGGAGCG 3′ |
| | Blautia / | | |
| | Ruminococcus | | |
| | (bacteria) | | |
| 40 | Eubacteria | DNA | 5′ ACTCCTACGGGAGGCAGCAGT 3′ |
| | (bacteria) | | |
| 41 | Eubacteria | DNA | 5′ ATTACCGCGGCTGCTGGC 3′ |
| | (bacteria) | | |

### REFERENCES

1. Y. Belkaid, T. W. Hand, Role of the microbiota in immunity and inflammation. Cell 157, 121-141 (2014).
2. J. V. Weinstock, D. E. Elliott, Helminths and the IBD hygiene hypothesis. Inflamm Bowel Dis 15, 128-133 (2009).
3. N. Kamada, G. Nunez, Regulation of the immune system by the resident intestinal bacteria. Gastroenterology 146, 1477-1488 (2014).
4. J. Jaenike, R. Unckless, S. N. Cockburn, L. M. Boelio, S. J. Perlman, Adaptation via symbiosis: recent spread of a Drosophila defensive symbiont. Science 329, 212-215 (2010).
5. C. G. Buffie et al., Precision microbiome reconstitution restores bile acid mediated resistance to Clostridium difficile. Nature 517, 205-208 (2015).
6. D. Ramanan, M. S. Tang, R. Bowcutt, P. Loke, K. Cadwell, Bacterial sensor Nod2 prevents inflammation of the small intestine by restricting the expansion of the commensal Bacteroides vulgatus. Immunity 41, 311-324 (2014).
7. I. Cleynen et al., Inherited determinants of Crohn's disease and ulcerative colitis phenotypes: a genetic association study. Lancet, (2015).
8. M. M. Hunter et al., In vitro-derived alternatively activated macrophages reduce colonic inflammation in mice. Gastroenterology 138, 1395-1405 (2010).
9. T. Ziegler et al., A novel regulatory macrophage induced by a helminth molecule instructs IL-10 in CD4+ T cells and protects against mucosal inflammation. J Immunol 194, 1555-1564 (2015).
10. L. Hang et al., Heligmosomoides polygyrus bakeri infection activates colonic Foxp3+ T cells enhancing their capacity to prevent colitis. J Immunol 191, 1927-1934 (2013).
11. M. M. Zaiss et al., The Intestinal Microbiota Contributes to the Ability of Helminths to Modulate Allergic Inflammation. Immunity 43, 998-1010 (2015).
12. J. E. Klementowicz, M. A. Travis, R. K. Grencis, Trichuris muris: a model of gastrointestinal parasite infection. Seminars in immunopathology 34, 815-828 (2012).
13. J. B. Holm et al., Chronic Trichuris muris Infection Decreases Diversity of the Intestinal Microbiota and Concomitantly Increases the Abundance of Lactobacilli. PLoS One 10, e0125495 (2015).
14. A. Houlden et al., Chronic Trichuris muris Infection in C57BL/6 Mice Causes Significant Changes in Host Microbiota and Metabolome: Effects Reversed by Pathogen Clearance. PLoS One 10, e0125945 (2015).
15. K. Atarashi et al., Treg induction by a rationally selected mixture of Clostridia strains from the human microbiota. Nature 500, 232-236 (2013).
16. R. D. Pridmore et al., The genome sequence of the probiotic intestinal bacterium Lactobacillus johnsonii NCC 533. Proc Natl Acad Sci USA 101, 2512-2517 (2004).
17. S. C. Lee et al., Helminth colonization is associated with increased diversity of the gut microbiota. PLoS Negl Trop Dis 8, e2880 (2014).
18. J. Nakayama et al., Diversity in gut bacterial community of school-age children in Asia. Scientific reports 5, 8397 (2015).
19. Z. D. Kurtz et al., Sparse and compositionally robust inference of microbial ecological networks. PLoS Comput Biol 11, e1004226 (2015).
20. D. Gevers et al., The treatment-naive microbiome in new-onset Crohn's disease. Cell Host Microbe 15, 382-392 (2014).
21. D. Gevers et al., The Human Microbiome Project: a community resource for the healthy human microbiome. PLoS Biol 10, e1001377 (2012).
22. AmGut, American Gut Project. Technical report.
23. U. M. Gundra et al., Alternatively activated macrophages derived from monocytes and tissue macrophages are phenotypically and functionally distinct. Blood 123, el10-122 (2014).
24. WHO, Guidelines for the evaluation of soil-transmitted helminthiasis and schistosomiasis at community level. A guide for managers of control programmes. Geneva: World Health Organization, (1998).
25. D. Wakelin, Acquired immunity to Trichuris muris in the albino laboratory mouse. Parasitology 57, 515-524 (1967).
26. K. J. Else, D. Wakelin, D. L. Wassom, K. M. Hauda, MHC-restricted antibody responses to Trichuris muris excretory/secretory (E/S) antigen. Parasite Immunol 12, 509-527 (1990).
27. R. M. Anthony et al., Memory T(H)2 cells induce alternatively activated macrophages to mediate protection against nematode parasites. Nat Med 12, 955-960 (2006).
28. D. Kim et al., TopHat2: accurate alignment of transcriptomes in the presence of insertions, deletions and gene fusions. Genome Biol 14, R36 (2013).
29. S. Anders, P. T. Pyl, W. Huber, HTSeq—a Python framework to work with high-throughput sequencing data. Bioinformatics 31, 166-169 (2015).
30. M. I. Love, W. Huber, S. Anders, Moderated estimation of fold change and dispersion for RNA-seq data with DESeq2. Genome Biol 15, 550 (2014).
31. J. G. Caporaso et al., Global patterns of 16S rRNA diversity at a depth of millions of sequences per sample. Proc Natl Acad Sci USA 108 Suppl 1, 4516-4522 (2011).
32. E. Aronesty. (2011).
33. J. Caporaso et al., QIIME allows analysis of high-throughput community sequencing data. Nat Methods 7, 335-336 (2010).
34. C. E. Shannon, A mathematical theory of communication. Bell System Technical Journal 27, 379-423, 623-656 (1948).
35. A. Chao, Nonparametric estimation of the number of classes in a population. Scandinavian Journal of statistics, 265-270 (1984).
36. J. Chen, K. Bittinger, E. Charlson, C. Hoffmann . . . , Associating microbiome composition with environmental covariates using generalized UniFrac distances. . . . , (2012).
37. C. Lozupone, M. E. Lladser, D. Knights, J. Stombaugh, R. Knight, UniFrac: an effective distance metric for microbial community comparison. The ISMS journal 5, 169-172 (2011).
38. Y. Vazquez-Baeza, M. Pirrung, A. Gonzalez, R. Knight, EMPeror: a tool for visualizing high-throughput microbial community data. GigaScience 2, 16 (2013).
39. N. Segata et al., Metagenomic biomarker discovery and explanation. Genome Biol 12, R60 (2011).
40. J. Aitchison, The statistical analysis of compositional data. Chapman and Hall, London New York, (1986).
41. D. Chung, S. Keles, Sparse partial least squares classification for high dimensional data. Stat Appl Genet Mol Biol 9, Article17 (2010).
42. H. Chun, S. Keles, Sparse partial least squares regression for simultaneous dimension reduction and variable selection. J R Stat Soc Series B Stat Methodol 72, 3-25 (2010).
43. D. R. Kim-Anh LêCao, Christèle Robert-Granié, and Philippe Besse., A sparse PLS for variable selection when integrating omics data. Statistical applications in genetics and molecular biology, 7, (2008).
44. H. Liu, K. Roeder, L. Wasserman, Stability Approach to Regularization Selection (StARS) for High Dimensional Graphical Models. Adv Neural Inf Process Syst 24, 1432-1440 (2010).
45. B. Liquet, K. A. Le Cao, H. Hocini, R. Thiebaut, A novel approach for biomarker selection and the integration of repeated measures experiments from two assays. BMC Bioinformatics 13, 325 (2012).
46. I. G. Kim-Anh Le Cao, Sebastien Dejean with key contributors Florian Rohart, Benoit Gautier, contributions from Pierre Monget, Jeff Coquery, FangZou Yao, and Benoit Liquet, mixOmics: Omics Data Integration Project. (2015).
47. R. K. Grencis, Immunity to helminths: resistance, regulation, and susceptibility to gastrointestinal nematodes. Annu Rev Immunol 33, 201-225 (2015).
1. A method for increasing the amount or activity of one or more bacterial species of the Clostridia class in the gastrointestinal tract of a subject comprising administering to said subject a therapeutically effective amount of a type 2 cytokine and/or a mucin.
2. A method for treating dysbiosis in the gastrointestinal tract of a subject in need thereof, wherein the dysbiosis is associated with a decrease in the amount or activity of one or more bacterial species of the Clostridia class in the gastrointestinal tract of the subject, said method comprising administering to said subject a therapeutically effective amount of a type 2 cytokine and/or a mucin.
3. A method for treating a gastrointestinal or inflammatory disorder in a subject in need thereof, which disorder can be treated by increasing the amount or activity of one or more bacterial species of the Clostridia class in the gastrointestinal tract of the subject, said method comprising administering to said subject a therapeutically effective amount of a type 2 cytokine and/or a mucin.
4. A method for promoting a wound healing in the gastrointestinal tract of a subject in need thereof comprising administering to said subject a therapeutically effective amount of a type 2 cytokine and/or a mucin.
5. The method of any one of items 1-4, further comprising administering to said subject bacteria of the Clostridia class.
6. The method of item 5, wherein said bacteria of the Clostridia class are from one or more different species.
7. The method of item 5 or item 6, wherein said bacteria of the Clostridia class are administered in the form selected from the group consisting of live bacterial cells, conditionally lethal bacterial strains, killed bacterial cells, spores, and bacterially-derived products.
8. The method of any one of items 5-7, wherein said bacteria of the Clostridia class are administered together with (i) a carrier and/or buffering agent and/or (ii) one or more prebiotic agents which enhance growth or activity of said bacteria.
9. The method of any one of items 5-8, wherein (i) said type 2 cytokine and/or a mucin and (ii) said bacteria of the Clostridia class are administered simultaneously.
10. The method of item 9, wherein (i) said type 2 cytokine and/or a mucin and (ii) said bacteria of the Clostridia class are administered in one composition.
11. The method of any one of items 5-8, wherein (i) said type 2 cytokine and/or a mucin and (ii) said bacteria of the Clostridia class are administered sequentially.
12. The method of any one of items 5-11, wherein said bacteria of the Clostridia class are from one or more human-derived commensal bacterial species belonging to Clostridium Cluster IV, XIVa, or XVIII.
13. The method of any one of items 5-11, wherein said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense.
14. The method of any one of items 5-11, wherein said bacteria of the Clostridia class are from one or more species selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLGO55, Erysipelotrichaceae bacterium 2_2_44A, Clostridium indolis, Anaerostipes caccae, Clostridium bolteae, Lachnospiraceae bacterium DJF_VP30, Lachnospiraceae bacterium 3_1_57 FAA_CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662.
15. The method of any one of items 5-11, wherein said bacteria of the Clostridia class are from one or more species selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLGO55, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA_CT1, Clostridium sp. 316002/08, and Clostridiales bacterium 1_7_47 FAA.
16. The method of any one of items 1-15, wherein the type 2 cytokine is selected from the group consisting of IL-13, IL-4, IL-22, IL-25, IL-33, and thymic stromal lymphopoietin (TSLP).
17. The method of any one of items 1-16, wherein the type 2 cytokine is a fusion protein comprising an amino acid sequence of a mature type 2 cytokine protein and CH₂ and CH₃ domains of Fc region of IgG.
18. The method of item 17, wherein the fusion protein consists of the amino acid sequence selected from the group consisting of:

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTERSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

19. The method of any one of items 1-18, wherein the method comprises administering two or more type 2 cytokines.
20. The method of item 19, wherein said two or more type 2 cytokines are selected from the group consisting of IL-13, IL-4, IL-22, IL-25, IL-33, and thymic stromal lymphopoietin (TSLP).
21. The method of item 19 or item 20, wherein said two or more type 2 cytokines are administered simultaneously.
22. The method of item 21, wherein said two or more type 2 cytokines are administered in one composition.
23. The method of item 19 or item 20, wherein said two or more type 2 cytokines are administered sequentially.
24. The method of any one of items 1-23, wherein the mucin comprises one or more molecules selected from the group consisting of MUC1, MUC2, MUC3A, MUC3B, MUC4, MUCSAC, MUC5B, MUC6, MUC7, MUC8, MUC12, MUC13, MUC15, MUC16, MUC17, MUC19, MUC20, and MUC21.
25. The method of item 24, wherein the mucin comprises MUC2.
26. The method of any one of items 1-25, wherein the method comprises administering a type 2 cytokine and a mucin.
27. The method of item 26, wherein the type 2 cytokine and the mucin are administered simultaneously.
28. The method of item 27, wherein the type 2 cytokine and the mucin are administered in one composition
29. The method of item 26, wherein the type 2 cytokine and the mucin are administered sequentially.
30. The method of any one of items 1-29, wherein the therapeutically effective amount of type 2 cytokine and/or mucin corresponds to the minimum dose required for the induction of M2 macrophages in the gastrointestinal tract of the subject.
31. The method of item 30, wherein the induction of M2 macrophages in the gastrointestinal tract of the subject is detected by monitoring the expression of one or more of PD-L2, CD301, CD206, Arg1, Ym1/Chi3l3, and Fizz1/Relma.
32. The method of any one of items 1-29, wherein the therapeutically effective amount of type 2 cytokine and/or mucin corresponds to the minimum dose required for the decrease of Bacteroides vulgatus abundance in the stool of the subject.
33. The method of item 32, wherein the therapeutically effective amount of type 2 cytokine and/or mucin corresponds to the minimum dose required for at least 90% decrease of Bacteroides vulgatus abundance in the stool of the subject.
34. The method of item 33, wherein the therapeutically effective amount of type 2 cytokine and/or mucin corresponds to the minimum dose required for at least 99% decrease of Bacteroides vulgatus abundance in the stool of the subject.
35. The method of any one of items 1-29, wherein the therapeutically effective amount of type 2 cytokine and/or mucin corresponds to the minimum dose required for the increase of the abundance of said Clostridial species in the stool of the subject.
36. The method of item 35, wherein the therapeutically effective amount of type 2 cytokine and/or mucin corresponds to the minimum dose required for at least 100% increase of the abundance of said Clostridial species in the stool of the subject.
37. The method of any one of items 1-36, wherein the type 2 cytokine and/or mucin is administered by a route selected from the group consisting of orally, rectally, fecally, and via naso/oro-gastric gavage.
38. The method of item 37, wherein the type 2 cytokine and/or mucin is administered in the form of nanoparticles.
39. The method of item 37, wherein the type 2 cytokine and/or mucin is administered in the form of a bacterial, yeast or viral strain engineered to produce such type 2 cytokine and/or mucin.
40. The method of item 39, wherein said bacterial strain is a Lactobacillus strain.
41. The method of any one of items 1-36, wherein the type 2 cytokine and/or mucin is administered systemically.
42. The method of any one of items 1-41, wherein the bacterial species of the Clostridia class is human-derived commensal bacterial species belonging to Clostridium Cluster IV, XIVa, or XVIII.
43. The method of any one of items 1-41, wherein the bacterial species of the Clostridia class is selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense.
44. The method of any one of items 1-41, wherein the bacterial species of the Clostridia class is selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLGO55, Erysipelotrichaceae bacterium 2_2_44A, Clostridium indolis, Anaerostipes caccae, Clostridium bolteae, Lachnospiraceae bacterium DJF_VP30, Lachnospiraceae bacterium 3_1_57 FAA_CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662.
45. The method of any one of items 1-41, wherein the bacterial species of the Clostridia class is selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLGO55, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA CT1, Clostridium sp. 316002/08, and Clostridiales bacterium 1_7_47 FAA.
46. The method of item 3, wherein the disorder is inflammatory bowel disease (IBD), ulcerative colitis, or Crohn's disease.
47. The method of item 3, wherein the disorder is selected from the group consisting of inflammatory bowel disease (IBD), ulcerative colitis, Crohn's disease, irritable bowel syndrome (IBS), sprue, autoimmune arthritis, rheumatoid arthritis, Type I diabetes, multiple sclerosis (MS), amyotrophic lateral sclerosis (ALS), graft vs. host disease, osteoarthritis, juvenile chronic arthritis, Lyme arthritis, psoriatic arthritis, reactive arthritis, spondy loarthropathy, systemic lupus erythematosus (SLE), insulin dependent diabetes mellitus, thyroiditis, asthma, psoriasis, dermatitis scleroderma, atopic dermatitis, graft versus host disease, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, septic shock, toxic shock syndrome, sepsis syndrome, cachexia, acquired immunodeficiency syndrome, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrome, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondy-loarhopathy, atheromatous disease/arteriosclerosis, allergic colitis, atopic allergy, food allergies such as peanut allergy, tree nut allergy, egg allergy, milk allergy, soy allergy, wheat allergy, seafood allergy, shellfish allergy, or sesame seed allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondy litis associated lung disease, vasculitic diffuse lung disease, haemosiderosis associated lung disease, drug-induced interstitial lung disease, radiation fibrosis, bronchiolitis obliterans, idiopathic pulmonary fibrosis, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, multiple sclerosis (all subtypes), insulindependent diabetes mellitus, sympathetic ophthalmia, pulmonary hypertension secondary to connective tissue disease, Goodpasture's syndrome, pulmonary manifestation of polyarteritis nodosa, acute rheumatio fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, allergic rhinitis (pollen allergies), anaphylaxis, pet allergies, latex allergies, drug allergies, allergic rhinoconjuctivitis, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis, diarrhea, colon cancer, cystic fibrosis, celiac disease, Type 2 diabetes, and autism-related immunopathologies.
48. The method of any one of items 1-47, wherein the method further comprises administering an effective amount of one or more Helminth species.
49. The method of item 48, wherein the Helminth species is selected from the group consisting of Trichuris trichiura, Schistosoma mansoni, Ancylostoma duodenale, Necator americanus, Fasciola hepatica, and Taenia saginata.
50. The method of any one of items 1-49, wherein the subject is human.
51. A composition comprising two or more components selected from the group consisting of (i) a type 2 cytokine, (ii) a mucin, (iii) bacteria of the Clostridia class, (iv) bacteria, yeast or virus expressing a type 2 cytokine, (v) yeast or virus expressing a mucin, and (vi) a helminth.
52. The composition of item 51, wherein said bacteria of the Clostridia class are from one or more different species.
53. The composition of item 51 or item 52, wherein said bacteria of the Clostridia class are in the form selected from the group consisting of live bacterial cells, conditionally lethal bacterial strains, killed bacterial cells, spores, and bacterially derived products.
54. The composition of any one of items 51-53, wherein said bacteria of the Clostridia class are from one or more human-derived commensal bacterial species belonging to Clostridium Cluster IV, XIVa, or XVIII.
55. The composition of any one of items 51-53, wherein said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense.
56. The composition of any one of items 51-53, wherein said bacteria of the Clostridia class are from one or more species selected from the group consisting of: Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57 FAA, Lachnospiraceae bacterium 6_1_63 FAA, Clostridium sp. 14616, Clostridium bolteae ATCC BAA-613, cf. Clostridium sp. MLGO55, Erysipelotrichaceae bacterium 2_2_44A, Clostridium indolis, Anaerostipes caccae, Clostridium bolteae, Lachnospiraceae bacterium DJF_VP30, Lachnospiraceae bacterium 3_1_57 FAA CT1, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47 FAA, Blautia cocoides, and Anaerostipes caccae DSM 14662.
57. The composition of any one of items 51-53, wherein said bacteria of the Clostridia class are from one or more species selected from the group consisting of: Clostridium saccharogumia, Clostridium ramosum JCM1298, Flavonifractor plautii, Pseudoflavonifractor capillosus ATCC 29799, Clostridium hathewayi, Clostridium saccharolyticum WM1, Bliautia coccoides, Lachnospiraceae bacterium 6_1_63 FAA, Clostridium sp. Clostridium bolteae ATCC BAA-613, cf. Clostridium sp. MLGO55, Erysipelotrichaceae bacterium 2_2_44A, Clostridium indolis, Anaerostipes caccae DSM 14662, Anaerotruncus colihominis, Anaerotruncus colihominis DSM 17241, Ruminococcus sp. ID8, Lachnospiraceae bacterium 2_1_46 FAA, Clostridium lavalense, Clostridium asparagiforme DSM 15981, Clostridium symbiosum, Clostridium symbiosum WAL-14163, Clostridium ramosum, Eubacterium contortum, Clostridium sp. D5, Clostridium scindens, Lachnospiraceae bacterium 5_1_57 FAA, Lachnospiraceae bacterium A4, Lachnospiraceae bacterium 3_1_57 FAA CT1, Clostridium sp. 316002/08, and Clostridiales bacterium 1_7_47 FAA.
58. The composition of item 51, wherein the type 2 cytokine is selected from the group consisting of IL-13, IL-4, IL-22, IL-25, IL-33, and thymic stromal lymphopoietin (TSLP).
59. The composition of item 51 or item 58, wherein the type 2 cytokine is a fusion protein comprising an amino acid sequence of a mature type 2 cytokine protein and CH₂ and CH₃ domains of Fc region of IgG.

60. The composition of item 59, wherein the fusion protein consists of the amino acid sequence selected from the group consisting of:

IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTFRSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

61. The composition of any one of items 51 and 58-60, wherein the composition comprises two or more type 2 cytokines.
62. The composition of item 61, wherein said two or more type 2 cytokines are selected from the group consisting of IL-13, IL-4, IL-22, IL-25, IL-33, and thymic stromal lymphopoietin (TSLP).

63. The composition of item 51, wherein the mucin comprises one or more molecules selected from the group consisting of MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, and MUC21.

64. The composition of item 51, wherein the mucin comprises MUC2.

65. The composition of any one of items 51 and 58-64, wherein the type 2 cytokine and/or mucin is associated with nanoparticles.

66. The composition of any one of items 51 and 58-64, wherein the bacteria expressing type 2 cytokine is a Lactobacillus strain.

67. The composition of item 51, wherein the helminth species is selected from the group consisting of Trichuris trichiura, Schistosoma mansoni, Ancylostoma duodenale, Necator americanus, Fasciola hepatica, and Taenia saginata.

68. The composition of any one of items 51-67, further comprising (i) a carrier and/or buffering agent and/or (ii) one or more prebiotic agents which enhance growth or activity of one or more bacteria present in the composition.

69. The composition of any one of items 51-68, wherein said composition is formulated for an administration route selected from the group consisting of oral, rectal, fecal, and via naso/oro-gastric gavage.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for increasing the amount or activity of one or more bacterial species of the Clostridia class in the gastrointestinal tract of a subject comprising administering to said subject a therapeutically effective amount of at least one type 2 cytokine and/or at least one mucin.

2. A method for treating dysbiosis in the gastrointestinal tract of a subject in need thereof, wherein the dysbiosis is associated with a decrease in the amount or activity of one or more bacterial species of the Clostridia class in the gastrointestinal tract of the subject, said method comprising administering to said subject a therapeutically effective amount of at least one type 2 cytokine and/or at least one mucin.

3. A method for treating a gastrointestinal or inflammatory disorder in a subject in need thereof, which disorder can be treated by increasing the amount or activity of one or more bacterial species of the Clostridia class in the gastrointestinal tract of the subject, said method comprising administering to said subject a therapeutically effective amount of at least one type 2 cytokine and/or at least one mucin.

4. A method for promoting a wound healing in the gastrointestinal tract of a subject in need thereof comprising administering to said subject a therapeutically effective amount of the at least one type 2 cytokine and/or the at least one mucin.

5. The method of claim 1, further comprising administering to said subject bacteria of the Clostridia class.

6. The method of claim 5, wherein said bacteria of the Clostridia class are administered in the form selected from the group consisting of live bacterial cells, conditionally lethal bacterial strains, killed bacterial cells, spores, and bacterially-derived products.

7. The method of claim 5, wherein (i) said at least one type 2 cytokine and/or at least one mucin and (ii) said bacteria of the Clostridia class are administered simultaneously in separate compositions or in one composition.

8. The method of claim 5, wherein (i) said at least one type 2 cytokine and/or at least one mucin and (ii) said bacteria of the Clostridia class are administered sequentially.

9. The method of claim 5, wherein said bacteria of the Clostridia class are from one or more human-derived commensal bacterial species belonging to Clostridium Cluster IV, XIVa, or XVIII.

10. The method of claim 5, wherein said bacteria of the Clostridia class are from one or more species selected from the group consisting of: Clostridium sacchorogumia, Clostridium viride, Clostridium butyricicoccus, Clostridium anaerobacterium, Blautia luti, Blautia coccoides, Blautia producta, Anaerostipes hadrus, Ruminococcus albus, Clostridium symbiosum, species of the genus Erysipelatoclostridium, Clostridium oroticum, Clostridium scindens, Ruminococcus faecis, Clostridium saccharolyticum, and Clostridium aldenense.

11. The method of claim 1, wherein the at least one type 2 cytokine is selected from the group consisting of IL-13, IL-4, IL-22, IL-25, IL-33, and thymic stromal lymphopoietin (TSLP).

12. The method of claim 1, wherein the at least one type 2 cytokine is a fusion protein comprising an amino acid sequence of a mature type 2 cytokine protein and CH₂ and CH₃ domains of Fc region of IgG.

13. The method of claim 12, wherein the fusion protein consists of the amino acid sequence selected from the group consisting of:
IL-4 Fc (Pr00114-1.9)(SEQ ID NO: 1):
HIHGCDKNHLREIIGILNEVTGEGTPCTEMDVPNVLTATKNTTESELVCR
ASKVLRIFYLKHGKTPCLKKNSSVLMELQRLFRAFRCLDSSISCTMNESK
STSLKDFLESLKSIMQMDYSGGGGSVPRDCGCKPCICTVPEVSSVFIFPP
KPKDVLMISLTPKVTCVVVDISKDDPEVQFSWFVDDVEVHTAQTKPREEQ
INSTFRSVSELPILHQDWLNGKEFKCRVNSAAFPAPIEKTISKTKGRPKA
PQVYTIPPPKEQMAKDKVSLTCMITNFFPEDITVEWQWNGQPAENYKNTQ
PIMDTDGSYFVYSKLNVQKSNWEAGNTFTCSVLHEGLHNHHTEKSLSHSP
GAHHHHHH;
IL-13 Fc (Pr00118-1.9)(SEQ ID NO: 2):
PVPRSVSLPLTLKELIEELSNITQDQTPLCNGSMVWSVDLAAGGFCVALD
SLTNISNCNAIYRTQRILHGLCNRKAPTTVSSLPDTKIEVAHFITKLLSY
TKQLFRHGPFGGGGSVPRDCGCKPCICTVPEVSSVFIFPPKPKDVLMISL
TPKVTCVVVDISKDDPEVQFSWFVDDVEVHTAQTKPREEQINSTFRSVSE
LPILHQDWLNGKEFKCRVNSAAFPAPIEKTISKTKGRPKAPQVYTIPPPK
EQMAKDKVSLTCMITNFFPEDITVEWQWNGQPAENYKNTQPIMDTDGSYF
VYSKLNVQKSNWEAGNTFTCSVLHEGLHNHHTEKSLSHSPGAHHHHHH,
and
IL-33 Fc (Pr00119-1.9)(SEQ ID NO: 3):
SIQGTSLLTQSPASLSTYNDQSVSFVLENGCYVINVDDSGKDQEQDQVLL
RYYESPCPASQSGDGVDGKKLMVNMSPIKDTDIWLHANDKDYSVELQRGD
VSPPEQAFFVLHKKSSDFVSFECKNLPGTYIGVKDNQLALVEEKDESCNN
IMFKLSKIGGGGSVPRDCGCKPCICTVPEVSSVFIFPPKPKDVLMISLTP
KVTCVVVDISKDDPEVQFSWFVDDVEVHTAQTKPREEQINSTFRSVSELP
ILHQDWLNGKEFKCRVNSAAFPAPIEKTISKTKGRPKAPQVYTIPPPKEQ
MAKDKVSLTCMITNFFPEDITVEWQWNGQPAENYKNTQPIMDTDGSYFVY
SKLNVQKSNWEAGNTFTCSVLHEGLHNHHTEKSLSHSPGAHHHHHH.

14. The method of claim 1, wherein the at least one mucin comprises one or more molecules selected from the group consisting of MUC1, MUC2, MUC3A, MUC3B, MUC4, MUC5AC, MUC5B, MUC6, MUC7, MUC8, MUC12, MUC13, MUC15, MUC16, MUC17, MUC19, MUC20, and MUC21.

15. The method of claim 14, wherein the mucin comprises MUC2.

16. The method of claim 1, wherein the therapeutically effective amount of the at least one type 2 cytokine and/or at least one mucin corresponds to the minimum dose required for the induction of M2 macrophages in the gastrointestinal tract of the subject.

17. The method of claim 1, wherein the therapeutically effective amount of the at least one type 2 cytokine and/or the at least one mucin corresponds to the minimum dose required for the decrease of Bacteroides vulgatus abundance in the stool of the subject or the minimum dose required for the increase of the abundance of said Clostridial species in the stool of the subject.

18. The method of claim 1, wherein the bacterial species of the Clostridia class is belongs to Clostridium Cluster IV, XIVa, or XVIII.

19. The method of claim 1, wherein the method further comprises administering an effective amount of one or more Helminth species.

20. A composition comprising two or more components selected from the group consisting of (i) at least one type 2 cytokine, (ii) at least one mucin, (iii) bacteria of the Clostridia class, (iv) bacteria, yeast or virus expressing a type 2 cytokine, (v) yeast or virus expressing a mucin, and (vi) a helminth.


SEQUENCE LISTING (41 sequences)

SEQ ID NO: 1 - protein, 358 aa, Artificial Sequence
  Description of Artificial Sequence Synthetic polypeptide
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGGGGSVPRDCGCKPCICTVPEVSSVFIFPPKPKDVLMISLTPKVTCVVVDISKDDPEVQFSWFVDDVEVHTAQTKPREEQINSTFRSVSELPILHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGAHHHHHH

SEQ ID NO: 2 - protein, 348 aa, Artificial Sequence
  Description of Artificial Sequence Synthetic polypeptide
PVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPFGGGGSVPRDCGCKPCICTVPEVSSVFIFPPKPKDVLMISLTPKVTCVVVDISKDDPEVQFSWFVDDVEVHTAQTKPREEQINSTFRSVSELPILHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGAHHHHHH

SEQ ID NO: 3 - protein, 396 aa, Artificial Sequence
  Description of Artificial Sequence Synthetic polypeptide
SIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKIGGGGSVPRDCGCKPCICTVPEVSSVFIFPPKPKDVLMISLTPKVTCVVVDISKDDPEVQFSWFVDDVEVHTAQTKPREEQINSTFRSVSELPILHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGAHHHHHH

SEQ ID NO: 4 - DNA, 60 bp, Artificial Sequence
  Description of Artificial Sequence Synthetic primer
aatgatacggcgaccaccgagatctacactatggtaattgtgtgccagcmgccgcggtaa

SEQ ID NO: 5 - DNA, 68 bp, Artificial Sequence
  Description of Artificial Sequence Synthetic primer
  modified_base (25)..(36): a, c, t, g, unknown or other
caagcagaagacggcatacgagatnnnnnnnnnnnnagtcagtcagccggactachvgggtwtctaat

SEQ ID NO: 6 - DNA, 22 bp, Artificial Sequence
  Description of Artificial Sequence Synthetic primer
agaccgcataggtaaagatacc

SEQ ID NO: 7 - DNA, 19 bp, Artificial Sequence
  Description of Artificial Sequence Synthetic primer
atgcgccataagtccatcc

SEQ ID NO: 8 - DNA, 17 bp, Artificial Sequence
  Description of Artificial Sequence Synthetic primer
aacggggtgctcatgac

SEQ ID NO: 9 - DNA, 21 bp, Artificial Sequence
  Description of Artificial Sequence Synthetic primer
cggtattagcactcctttcgg

SEQ ID NO: 10 - DNA, 17 bp, Artificial Sequence
  Description of Artificial Sequence Synthetic primer
cgagcgaagcggtttca

SEQ ID NO: 11 - DNA, 24 bp, Artificial Sequence
  Description of Artificial Sequence Synthetic primer
ttctaactgttatcccccagtgta

SEQ ID NO: 12 - DNA, 18 bp, Artificial Sequence
  Description of Artificial Sequence Synthetic primer
gagcgaagcagtaagacg

SEQ ID NO: 13 - DNA, 23 bp, Artificial Sequence
  Description of Artificial Sequence Synthetic primer
ctaactgttatccccctgtatga

SEQ ID NO: 14 - DNA, 17 bp, Artificial Sequence
  Description of Artificial Sequence Synthetic primer
cggcgtgcctaacacat

SEQ ID NO: 15 - DNA, 20 bp, Artificial Sequence
  Description of Artificial Sequence Synthetic primer
gtccgccactcagtcaatca

SEQ ID NO: 16 - DNA, 21 bp, Artificial Sequence
  Description of Artificial Sequence Synthetic primer
gtcgaacgaagtgaagatagc

SEQ ID NO: 17 - DNA, 19 bp, Artificial Sequence
  Description of Artificial Sequence Synthetic primer
ttatcccggtcatacaggc

SEQ ID NO: 18 - DNA, 20 bp, Artificial Sequence
  Description of Artificial Sequence Synthetic primer
tggggaacctgccctataca

SEQ ID NO: 19 - DNA, 20 bp, Artificial Sequence
  Description of Artificial Sequence Synthetic primer
cggagcttttcacaccgaat

SEQ ID NO: 20 - DNA, 19 bp, Artificial Sequence
  Description of Artificial Sequence Synthetic primer
acggagcttacgttttgaa

SEQ ID NO: 21 - DNA, 19 bp, Artificial Sequence
  Description of Artificial Sequence Synthetic primer
ggctgttatccccctctga

SEQ ID NO: 22 - DNA, 19 bp, Artificial Sequence
  Description of Artificial Sequence Synthetic primer
gcgctgttttcagaatctt

SEQ ID NO: 23 - DNA, 20 bp, Artificial Sequence
  Description of Artificial Sequence Synthetic primer
accggagtttttcacactac

SEQ ID NO: 24 - DNA, 17 bp, Artificial Sequence
  Description of Artificial Sequence Synthetic primer
cggcgtgcctaacacat

SEQ ID NO: 25 - DNA, 21 bp, Artificial Sequence
  Description of Artificial Sequence Synthetic primer
cgccactcagtcatctcagaa

SEQ ID NO: 26 - DNA, 21 bp, Artificial Sequence
  Description of Artificial Sequence Synthetic primer
agtcgaacgaagcgatttaac

SEQ ID NO: 27 - DNA, 21 bp, Artificial Sequence
  Description of Artificial Sequence Synthetic primer
ccggagtttttcacactgtat

SEQ ID NO: 28 - DNA, 17 bp, Artificial Sequence
  Description of Artificial Sequence Synthetic primer
tcgaacgcgagcacttg

SEQ ID NO: 29 - DNA, 18 bp, Artificial Sequence
  Description of Artificial Sequence Synthetic primer
ccatgcagtgtccgtacc

SEQ ID NO: 30 - DNA, 22 bp, Artificial Sequence
  Description of Artificial Sequence Synthetic primer
gcgctttacttagatttcttcg

SEQ ID NO: 31 - DNA, 17 bp, Artificial Sequence
  Description of Artificial Sequence Synthetic primer
ccatgcggtactgtggt

SEQ ID NO: 32 - DNA, 16 bp, Artificial Sequence
  Description of Artificial Sequence Synthetic primer
ggagatgaaggcggct

SEQ ID NO: 33 - DNA, 16 bp, Artificial Sequence
  Description of Artificial Sequence Synthetic primer
accctctcaggtcggc

SEQ ID NO: 34 - DNA, 19 bp, Artificial Sequence
  Description of Artificial Sequence Synthetic primer
gcagtcgaacggagttatg

SEQ ID NO: 35 - DNA, 19 bp, Artificial Sequence
  Description of Artificial Sequence Synthetic primer
cacactgcctcatgtgaag

SEQ ID NO: 36 - DNA, 23 bp, Artificial Sequence
  Description of Artificial Sequence Synthetic primer
cagtcgaacgaagcatcttatag

SEQ ID NO: 37 - DNA, 19 bp, Artificial Sequence
  Description of Artificial Sequence Synthetic primer
gatccatctcacaccacct

SEQ ID NO: 38 - DNA, 19 bp, Artificial Sequence
  Description of Artificial Sequence Synthetic primer
ggtgtaggtgggtatggac

SEQ ID NO: 39 - DNA, 20 bp, Artificial Sequence
  Description of Artificial Sequence Synthetic primer
aaatcctctttacaggagcg

SEQ ID NO: 40 - DNA, 21 bp, Artificial Sequence
  Description of Artificial Sequence Synthetic primer
actcctacgggaggcagcagt

SEQ ID NO: 41 - DNA, 18 bp, Artificial Sequence
  Description of Artificial Sequence Synthetic primer
attaccgcggctgctggc